(19) United States
(12) Patent Application Publication
(10) Pub. No.: US 2014/0066469 A1
(43) Pub. Date: Mar. 6, 2014

(54) TREATMENT OF DISEASES ASSOCIATED WITH INFLAMMATION

(71) Applicants: Department of Veterans Affairs, Washington, DC (US); The Board of Trustees of the Leland Stanford Junior University, Palo Alto, CA (US)

(72) Inventors: William H. Robinson, Palo Alto, CA (US); Jeremy Sokolove, Mountain View, CA (US); Qian Wang, Palo Alto, CA (US); Heidi H. Wong, San Francisco, CA (US); Mark C. Genovese, Sunnyvale, CA (US)

(21) Appl. No.: 13/972,614

(22) Filed: Aug. 21, 2013

Related U.S. Application Data
(60) Provisional application No. 61/691,582, filed on Aug. 21, 2012.
(60) Provisional application No. 61/792,404, filed on Mar. 15, 2013.

(51) Int. Cl. A61K 31/4706; A61K 45/06; A61K 31/40

(52) U.S. Cl. CPC A61K 31/4706; A61K 31/40; A61K 45/06
USPC 514/275; 514/313

(57) ABSTRACT

Compositions and methods are provided for preventing or treating the pre-clinical early-stages of inflammatory diseases, including autoimmune diseases, degenerative inflammatory diseases, metabolic inflammatory diseases, chronic infection associated with inflammation, cancer associated with inflammation, and other inflammatory diseases by administration to an individual of an effective dose of a synergistic combination of active agents comprising or consisting essentially of an aminoquinoline, e.g. hydroxychloroquine, and a statin, e.g. atorvastatin. Each or both of the active agents can be formulated in various ways, including without limitation a solid oral dosage form.

### GOVERNMENT RIGHTS

This invention was made with Government support under contracts A1069160 and HV000242 awarded by the National Institutes of Health. The Government has certain rights in this invention.

### FIELD OF THE INVENTION

The present invention relates to the prevention and treatment of inflammation, pain, and tissue damage. In particular, the present invention relates to use of combination therapies described below as a composition and method for treating inflammatory diseases, including arthritis, demyelinating diseases, degenerative diseases, infectious diseases, metabolic diseases, cardiovascular diseases, cancer, and other diseases associated with inflammation. The present invention also relates to the prevention and treatment of such inflammatory diseases, including preventing the onset of disease in individuals at high risk for developing the disease and preventing the progression of disease in individuals at early stages of the disease.

### BACKGROUND OF THE INVENTION

The invention relates to the treatment of diseases in which inflammation contributes to pathogenesis, including autoimmune diseases, including rheumatoid arthritis (RA) and multiple sclerosis (MS); degenerative diseases with an inflammatory component, such as osteoarthritis (OA), Alzheimer's disease (AD), and macular degeneration; chronic infections, such as HIV; as well as other inflammatory conditions, such as hepatic inflammation, cardiovascular diseases, metabolic diseases, and cancers.

### Aminoquinolines and Hydroxychloroquine

Aminoquinolines are derivatives of quinoline that are most notable for their roles as antimalarial drugs but also possess anti-inflammatory properties. Examples of the aminoquinoline class include, but are not limited to, 4-aminoquinolines, such as amodiaquine, hydroxychloroquine, chloroquine; and 8-aminoquinolines, such as primaquine and pamaquine. 4-Aminoquinoline is a form of aminoquinoline with the amino group at the 4-position of the quinoline. A variety of derivatives of 4-aminoquinoline are antimalarial agents, and examples include amodiaquine, chloroquine, and hydroxychloroquine. The drugs may be formulated as a base, or more usually as a salt.

The 4-aminoquinoline hydroxychloroquine (HCQ) was initially developed as hydroxychloroquine sulfate (HCQ sulfate) for use as an antimalarial drug. Hydroxychloroquine sulfate is sold under the trade names Plaquenil™, Axemal™ (in India), Dolquine™, and Quensyl™, and is also widely used to reduce inflammation in the treatment of systemic lupus erythematosus, rheumatoid arthritis, Sjögren's Syndrome, and porphyria cutanea tarda. HCQ sulfate has also provided anti-inflammatory benefit in chronic HIV infection and in type II diabetes. HCQ sulfate is also used in the treatment of arthritis that develops following Lyme disease. It may have both an anti-spirochaete activity and an anti-inflammatory activity (Steere and Angelis (2006). Arthritis Rheum. 54 (10): 3079-86). HCQ differs from chloroquine by having a hydroxyl group at the end of the side chain: The N-ethyl substituent is beta-hydroxylated. It is available for oral administration as hydroxychloroquine sulfate (Plaquenil), of which 200 mg contains 155 mg hydroxychloroquine base in chiral form. In addition to 155 mg of hydroxychloroquine base, each Plaquenil tablet contains the following inactive ingredients: anhydrous lactose, croscarmellose sodium, glyceryl triacetate, hypromellose, magnesium stearate, microcrystalline cellulose, polydextrose, polyethylene glycol, povidone, sodium lauryl sulfate and titanium dioxide. Hydroxychloroquine sulfate has similar pharmacokinetics to chloroquine phosphate, being quickly absorbed by the gastrointestinal tract and eliminated by the kidney. Cytochrome P450 enzymes (CYP 2D6, 2C8, 3A4, and 3A5) N-desethylate HCQ to N-desethylhydroxychloroquine (Kalia et al. (2007) Dermatologic Therapy 20 (4): 160-174).

Hydroxychloroquine (HCQ) increases lysosomal pH in antigen-presenting cells, and this is believed to be a primary mechanism by which it exerts anti-inflammatory effects and alters toll-like receptor (TLR) activity (Waller et al. Medical pharmacology and therapeutics (2nd ed.). p. 370). HCQ inhibits TLRs on plasmacytoid dendritic cells, macrophage and other cells. Activation of TLR 9, a TLR that recognizes DNA-containing immune complexes, leads to the production of interferon and causes the dendritic cells to mature and present antigen to T cells. HCQ, by decreasing TLR 9 signaling, reduces the activation of dendritic cells and hence the inflammatory process.

Toxicity of HCQ.

The most common adverse effects of HCQ therapy are mild nausea and occasional stomach cramps with mild diarrhea. The most serious adverse effects affect the eye. During prolonged HCQ treatment of lupus or arthritis, adverse effects can include these adverse symptoms, plus altered eye pigmentation. One of the most serious side effects of chronic HCQ use is ocular toxicity (Flach (2007). Transactions of the American Ophthalmological Society 105: 191-4; discussion 195-7). The daily safe maximum dose for eye toxicity can be estimated based on one's height and weight.

Eye toxicity resulting from HCQ and other aminoquinoline use. Prolonged use of HCQ, chloroquine, or other aminoquinolines is associated with the development of eye toxicity. The incidence of such toxicity increases markedly with the duration of therapy, with ophthalmoscopically visualized loss of retinal pigmented epithelium in 1% of treated humans after 5 years; considerably higher rates of toxicity are observed with chloroquine. Notably, despite less than 1% of patients developing clinically apparent eye toxicity, total rates of physician discontinuation of HCQ for earlier eye problems (including asymptomatic changes noted on ophthalmologic examination) approach 7% of treated patients over 5 years (Marmor et al. Arthritis Care Res. 2010; 62(6):775-84).

Toxicity due to HCQ may occur in two distinct areas of the eye: the cornea and the macula. The cornea may become affected (relatively commonly) by an innocuous vortex keratopathy that is characterized by whorl-like corneal epithelial deposits. These changes bear no relationship to dosage and are usually reversible on cessation of HCQ. Changes to the macula (a component of the retina) are more serious and are related to dosage and duration of HCQ use. Advanced retinopathy is characterized by reduction of visual acuity and a “bull's-eye” macular lesion, which is absent in the earlier stages.

Macular retinal toxicity is related to the total cumulative dose rather than the daily dose. People taking 400 mg of HCQ sulfate or less per day generally have a negligible risk of macular retinal toxicity, but the risk begins to increase when a person takes the medication for more than 5 years or takes a cumulative dose of more than 1000 grams. Regular eye screening, even in the absence of visual symptoms, is recommended to begin when either of these risk factors is present (Marmor et al. (2011) Ophthalmology 118 (2): 415-22).

The exact mechanisms underlying HCQ-induced retinal toxicity, including retinal macular toxicity, are not clear. Studies to date have identified retinal accumulation of HCQ to levels much higher than those observed in other tissues and in the blood. In addition, HCQ binds to melanin in the retinal pigment epithelium (RPE), and such binding may contribute to or prolong HCQ's toxic effects. Some studies have demonstrated that both chloroquine and HCQ are associated with increased lipofuscin formation, a process known to be accelerated by increased lysosomal pH and intra-lysosomal oxidation during degradation of auto-/heterophagocytosed material (Sundelin et al. APMIS [Acta Pathologica, Microbiologica et Immunologica Scandinavica]. 2002; 110(6):481-9). Additionally, because melanin within the RPE has a role in neutralizing oxidative free radicals, it has been suggested that the presence of excessive levels of such free radicals may contribute to the pathogenesis of HCQ-induced retinal toxicity (Sundelin et al. APMIS. 2002; 110(6):481-9).

Retinal toxicity induced by chloroquine and HCQ is characterized by a fine mottling of the macula, arteriolar narrowing, peripheral retinal pigmentation, loss of the foveal reflex and, in advanced cases, by a depigmented macula surrounded by a pigmented ring, a finding termed “bull's-eye maculopathy” (Mecklenburg et al, Toxicol Pathol. 2007; 35(2):252-67). In the early stages of retinal toxicity, patients may notice decreased visual acuity, blurred vision, decreased color and night vision, as well as a paracentral scotoma (Mecklenburg et al, Toxicol Pathol. 2007; 35(2):252-67). HCQ retinopathy is dose related and develops slowly, but can progress to a more serious loss of central and peripheral vision for which there is no known treatment (Marmor et al. Arthritis Care Res. 2010; 62(6):775-84).

Current recommendations for screening for chloroquine and HCQ retinopathy are described in Marmor et al (Ophthalmology. 2011, 118(2):415-22). The recommendations include performing a baseline examination of patients starting these drugs to serve as a reference point and to rule out pre-existing maculopathy, which might contraindicate use of these drugs. Annual screening for eye toxicity should begin after 5 years (or sooner, if there are unusual risk factors). Newer objective tests, such as multifocal electroretinogram (mfERG), spectral domain optical coherence tomography (SD-OCT), and fundus autofluorescence (FAF), can be more sensitive than visual field tests. It is now recommended that along with 10-2 automated field tests, at least one of these procedures be used for routine screening where available. When field tests are performed independently, even the most subtle 10-2 field changes should be taken seriously and are an indication for evaluation by objective testing. Because mfERG testing is an objective test that evaluates function, it may be used in place of visual field tests. Amsler grid testing is no longer recommended. Fundus examinations are advised for documentation, but visible bull's-eye maculopathy is a late change, and the goal of screening is to detect toxicity at an earlier stage. Further, patients should be aware of the risk of toxicity and the rationale for screening (to detect early changes and minimize visual loss, not necessarily to prevent it). The drugs should be stopped if possible when toxicity is detected or strongly suspected, but this is a decision to be made in conjunction with patients and their medical physicians (Marmor et al, Ophthalmology. 2011, 118(2):415-22).

### Statins and Atorvastatin

Statins are inhibitors of HMG-CoA reductase, and are used to lower cholesterol levels to prevent and treat atherosclerosis and coronary artery disease. These agents are described in detail; for example, mevastatin and related compounds as disclosed in U.S. Pat. No. 3,983,140; lovastatin (mevinolin) and related compounds as disclosed in U.S. Pat. No. 4,231,938; pravastatin and related compounds as disclosed in U.S. Pat. No. 4,346,227; simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional agents and compounds are disclosed in U.S. Pat. Nos. 5,208,258, 5,130,306, 5,116,870, 5,049,696, RE 36,481, and RE 36,520. Statins include the salts and/or ester thereof.

Atorvastatin, marketed by Pfizer as a calcium salt under the trade name Lipitor, is a member of the drug class known as statins, used for lowering blood cholesterol. It also stabilizes plaque and prevents strokes through anti-inflammatory and other mechanisms. Like all statins, atorvastatin works by inhibiting HMG-CoA reductase, an enzyme found in liver tissue that plays a key role in production of cholesterol in the body. Atorvastatin calcium undergoes rapid absorption when taken orally, with an approximate time to maximum plasma concentration (Tmax) of 1-2 h. The absolute bioavailability of the drug is about 14%, but the systemic availability for HMG-CoA reductase activity is approximately 30%. Atorvastatin undergoes high intestinal clearance and first-pass metabolism, which is the main cause for the low systemic availability. Administration of atorvastatin calcium with food produces a 25% reduction in Cmax (rate of absorption) and a 9% reduction in AUC (extent of absorption), although food does not affect the plasma LDL-C-lowering efficacy of atorvastatin. Evening dose administration is known to reduce the Cmaxand AUC by 30% each. However, time of administration does not affect the plasma LDL-C-lowering efficacy of atorvastatin. Atorvastatin is highly protein bound (≧98%).

Lipitor Tablets for oral administration contain 10, 20, 40, or 80 mg atorvastatin base and the following inactive ingredients: calcium carbonate, USP; candelilla wax, FCC; croscarmellose sodium, NF; hydroxypropyl cellulose, NF; lactose monohydrate, NF; magnesium stearate, NF; microcrystalline cellulose, NF; Opadry White YS-1-7040 (hypromellose, polyethylene glycol, talc, titanium dioxide); polysorbate 80, NF; simethicone emulsion.

### Inflammatory Disease

Many diseases have an underlying inflammatory component that contributes to disease initiation and/or progression. Inflammatory diseases include autoimmune diseases, such rheumatoid arthritis (RA), Crohn's disease, psoriasis, systemic lupus erythematosus (SLE), and multiple sclerosis (MS); degenerative diseases, such as osteoarthritis (OA), Alzheimer's disease (AD), and macular degeneration; chronic infections, such as infection with human immunodeficiency virus (HIV), chronic hepatitis C virus (HCV), chronic hepatitis B virus (HBV), chronic cytomegalovirus (CMV), mycobacterium tuberculosis (TB), or other chronic viral and bacterial infections; inflammatory metabolic diseases, such as type II diabetes and hepatic disease; cardiovascular diseases, such as atherosclerosis; cancers, which can arise from and induce inflammation; as well as other diseases with an inflammatory component.

### Autoimmune Diseases

Rheumatoid Arthritis (RA). RA is a chronic syndrome characterized by usually symmetric inflammation of the peripheral joints. It may result in progressive destruction of articular and periarticular structures, with or without generalized manifestations (Firestein (2003) Nature 423(6937):356-61; McInnes and Schett. (2011) N Engl J. Med. 365(23):2205-19). Its cause is unknown. A genetic predisposition has been identified and, in some populations, localized to a pentapeptide in the HLA-DR beta1 locus of class II histocompatibility genes. Environmental factors may also play a role. For example, individuals who both smoke cigarettes and possess HLA-DR4 containing the “shared epitope” polymorphism have an approximately 10-20 fold greater risk of developing RA. Cigarette smoking is thought to induce anti-citrullinated protein antibody (ACPA) responses, which are measured using the commercial cyclic-citrullinated peptide (CCP) assay (Klareskog et al. (2006) Arthritis Rheum. 54(1):38-46). In addition, periodontitis and infection with P. gingivalis might also play a role in the initiation of autoimmune and anti-citrullinated protein antibody (ACPA) responses that result in development of RA (Rutger and Persson. (2012) J Oral Microbiol. 4). Immunologic changes may be initiated by multiple factors. About 0.6% of all populations are affected, women two to three times more often than men. Onset may be at any age, most often between 25 and 50 years of age.

Prominent immunologic abnormalities that may be important in the pathogenesis of RA include immune complexes found in joint tissues, in and in association with vasculitis. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased expression of adhesion molecules contribute to inflammatory cell migration to and retention in the synovial tissue. An increase in number of macrophage-derived lining cells, along with an increase in number of certain lymphocytes and changes in synovial vasculature, occur early in the disease process.

In chronically affected joints, the normally delicate synovium develops many villous folds and thickens because of an increase in the numbers and size of synovial lining cells and colonization by lymphocytes and plasma cells. The lining cells produce various materials, including collagenase and stromelysin, which can contribute to cartilage destruction; IL-1, which stimulates lymphocyte proliferation; and prostaglandins. The infiltrating cells, which are initially perivenular but later form lymphoid follicles with germinal centers, synthesize IL-2 and other cytokines, as well as rheumatoid factor (RF; antibodies to human γ-globulin) and other immunoglobulins. Fibrin deposition, fibrosis, and necrosis also are present. Hyperplastic synovial tissue (pannus) may erode cartilage, subchondral bone, articular capsule, and ligaments. Polymorphonuclear neutrophils are not prominent in the synovium but often predominate in the synovial fluid.

Onset is usually insidious, with progressive involvement of additional joints, but may also be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint. RA is characterized by focal bone erosions through degradation and remodeling of bone at the joint margins and in subchondral bone. A subset of RA patients develop specific autoantibodies, including RF and ACPA. RF are present in about 70% of patients with RA. However, RF, often in low titers, are also present in patients with other diseases, including other connective tissue diseases such as systemic lupus erythematous, granulomatous diseases, chronic infections such as viral hepatitis, subacute bacterial endocarditis, and tuberculosis, and cancers. Low RF titers are also present in a small percentage of the general population, more commonly in the elderly. The presence of ACPA, as detected by the clinical anti-CCP test, is approximately 60% sensitive and 95% specific for the diagnosis of RA, and as with RF, indicates a worse prognosis.

Systemic lupus erythematosus (SLE). SLE is a systemic autoimmune disease characterized by malar rashes, oral ulcers, photosensitivity, serositis, seizures, low white-blood-cell counts, low platelet counts, seizures, and the presence of anti-nuclear antibodies (ANA) and other autoantibodies. It is characterized by polyclonal B-cell activation, which results in production of a variety of autoantibodies that form immune complexes and thereby induce inflammation, which in turn contributes to tissue damage (see Kotzin et al. (1996) Cell 85:303-06 for a review of the disease). SLE has a variable course characterized by exacerbations and remissions and is difficult to study. For example, some patients may have predominantly skin rashes and joint pain, undergo spontaneous remission, and require little medication. Others may have severe and progressive kidney involvement (glomerulonephritis and cerebritis) that requires therapy with high doses of steroids and cytotoxic drugs, such as cyclophosphamide. HCQ slows SLE progression and is a mainstay therapeutic for the management of SLE.

Inflammatory bowel diseases. Inflammatory bowel diseases, including Crohn's disease and ulcerative colitis, involve autoimmune attack of the bowel. These diseases cause chronic diarrhea, frequently bloody, as well as symptoms of colonic dysfunction.

Systemic sclerosis (SSc, or scleroderma). SSc is an autoimmune disease characterized by fibrosis of the skin and internal organs and widespread vasculopathy. Patients with SSc are classified according to the extent of cutaneous sclerosis: patients with limited SSc have skin thickening of the face, neck, and distal extremities, whereas those with diffuse SSc have involvement and skin thickening of the trunk, abdomen, and proximal extremities as well. Involvement of internal organs tends to occur earlier in the course of disease in patients with diffuse compared with limited disease (Laing et al. (1997) Arthritis. Rheum. 40:734-42). Most patients with diffuse SSc who develop severe internal organ involvement will do so within the first three years after diagnosis, at the time the skin becomes progressively fibrotic (Steen and Medsger (2000) Arthritis Rheum. 43:2437-44.). Common manifestations of diffuse SSc that are responsible for substantial morbidity and mortality include interstitial lung disease, Raynaud's phenomenon and digital ulcerations, pulmonary arterial hypertension (Trad et al. (2006) Arthritis. Rheum. 54:184-91.), musculoskeletal symptoms, and heart and kidney involvement (Ostojic and Damjanov (2006) Clin. Rheumatol. 25:453-7). Current therapies focus on treating specific symptoms; disease-modifying agents targeting the underlying pathogenesis are lacking.

Multiple sclerosis (MS). MS is a debilitating, inflammatory, neurological illness characterized by demyelination of the central nervous system. The disease affects primarily young adults, more commonly women. Symptoms of the disease include fatigue, numbness, tremor, tingling, dysesthesias, visual disturbances, dizziness, cognitive impairment, urological dysfunction, decreased mobility, and depression. Four types classify the clinical patterns of the disease: relapsing-remitting, secondary-progressive, primary-progressive and progressive-relapsing (S. L. Hauser and D. E. Goodkin, Multiple Sclerosis and Other Demyelinating Diseases in Harrison's Principles of Internal Medicine 14th Edition, vol. 2, McGraw-Hill, 1998, pp. 2409-19).

### Degenerative Inflammatory Diseases

Many degenerative diseases have an underlying inflammatory component. Examples of such degenerative diseases include osteoarthritis (OA), Alzheimer's disease (AD), and macular degeneration.

Osteoarthritis (OA).

OA affects nearly 27 million people in the United States, accounting for 25% of visits to primary care physicians, and half of all prescriptions for non-steroidal anti-inflammatory drugs (NSAIDs). It is a chronic arthropathy characterized by disruption and potential loss of joint cartilage along with other joint changes, including bone remodeling such as bone hypertrophy (osteophyte formation), subchondral sclerosis, and formation of subchondral cysts. OA is viewed as failure of the synovial joint (Abramson et al, Arthritis Res Ther. 2009; 11(3):227; Krasnokutsky et al, Osteoarthritis Cartilage. 2008; 16 Suppl 3:S1-3; Brandt et al, Rheum Dis Clin North Am. 2008 August; 34(3):531-59). OA results in the degradation of joints, including degradation of articular cartilage and subchondral bone, resulting in mechanical abnormalities and joint dysfunction. Symptoms may include joint pain, tenderness, stiffness, sometimes an effusion, and impaired joint function. A variety of causes can initiate processes leading to loss of cartilage in OA. A subgroup of OA patients exhibit a form of OA termed “erosive OA”, which includes erosive changes in the involved joints, typically involves the hands, and is clinically-distinct from the more common and typical form of OA that does not involve erosive changes (Punzi L, Best Pract Res Clin Rheumatol. 2004 18(5):739-58); Belhorn L R, et.al. Semin Arthritis Rheum. 1993, 22(5):298-306). Although erosive OA has an inflammatory etiology, the studies described herein pertain to general non-erosive OA.

OA (the non-erosive and common form) may begin with joint damage caused by trauma to the joint; mechanical injury to the meniscus, articular cartilage, a joint ligament, or other joint structure; defects in cartilage matrix components; and the like. Mechanical stress on joints may underlie the development of OA in many individuals, with the sources of such mechanical stress being many and varied, including misalignment of bones as a result of congenital or pathogenic causes; mechanical injury; overweight; loss of strength in muscles supporting joints; and impairment of peripheral nerves, leading to sudden or dyscoordinated movements that overstress joints.

Articular cartilage comprises chondrocytes that generate and are surrounded by extracellular matrix. In synovial joints there are at least two movable bony surfaces that are surrounded by the synovial membrane, which secretes synovial fluid, a transparent alkaline viscid fluid that fills the joint cavity, and articular cartilage, which is interposed between the articulating bony surfaces. The earliest gross pathologic finding in OA is softening of the articular cartilage in habitually loaded areas of the joint surface. This softening or swelling of the articular cartilage is frequently accompanied by loss of proteoglycans from the cartilage matrix. As OA progresses, the integrity of the cartilage surface is lost and the articular cartilage thins, with vertical clefts extending into the depth of the cartilage in a process called fibrillation. Joint motion may cause fibrillated cartilage to shed segments and thereby expose the bone underneath (subchondral bone). In OA, the subchondral bone is remodeled, featuring subchondral sclerosis, subchondral cysts, and ectopic bone comprising osteophytes. The osteophytes (bone spurs) form at the joint margins, and the subchondral cysts may be filled with synovial fluid. The remodeling of subchondral bone increases the mechanical strain and stresses on both the overlying articular cartilage and the subchondral bone, leading to further damage of both the cartilage and subchondral bone.

The tissue damage stimulates chondrocytes to attempt repair by increasing their production of proteoglycans and collagen. However, efforts at repair also stimulate the enzymes that degrade cartilage, as well as inflammatory cytokines, which are normally present in only small amounts. Inflammatory mediators trigger an inflammatory cycle that further stimulates the chondrocytes and synovial lining cells, eventually breaking down the cartilage. Chondrocytes undergo programmed cell death (apoptosis) in OA joints.

OA is characterized by low-grade infiltration of inflammatory cells, primarily macrophages, but also B cells and T cells. These cells, again primarily macrophages, are capable of producing inflammatory cytokines and matrix metalloproteases (MMPs) in the OA joint. However, when stimulated by inflammatory cytokines, such as IL-1 and TNF, tissue-resident cells within the joint, including synovial fibroblasts and chondrocytes, can produce additional inflammatory cytokines, including IL-6, as well as multiple MMPs.

OA should be suspected in patients with gradual onset of joint symptoms and signs, particularly in older adults, usually beginning with one or a few joints. Pain can be the earliest symptom, sometimes described as a deep ache. Pain is usually worsened by weight bearing and relieved by rest but can eventually become constant. Joint stiffness in OA is associated with awakening or inactivity. If OA is suspected, plain x-rays should be taken of the most symptomatic joints. X-rays generally reveal marginal osteophytes, narrowing of the joint space, increased density of the subchondral bone, subchondral cyst formation, bony remodeling, and joint effusions. Standing x-rays of knees are more sensitive in detecting joint-space narrowing. Magnetic resonance imaging (MRI) can be used to detect cartilage degeneration, and several MRI-based based scoring systems exist for characterizing the severity of OA (Hunter et al, PM R. 2012 May; 4(5 Suppl):568-74).

OA commonly affects the hands, feet, spine, and the large weight-bearing joints, such as the hips and knees, although in theory any joint in the body can be affected. As OA progresses, the affected joints appear larger, are stiff and painful, and usually feel better with gentle use but worse with excessive or prolonged use. Treatment generally involves a combination of exercise, lifestyle modification, and analgesics. If pain becomes debilitating, joint-replacement surgery may be used to improve quality of life.

Among the agents proposed to modify disease in OA—such as doxycycline (presumably through its ability to inhibit MMPs), bisphosphonates (presumably through their ability to inhibit osteoclast activation), and licofelone (presumably through its ability to inhibit the cyclooxygenase and lipoxegenase pathways)—none have been shown to afford robust chondroprotection as defined by slowing of cartilage breakdown. Among the agents that have demonstrated partial efficacy in controlling OA-associated pain are analgesics such as acetaminophen and anti-inflammatories such as NSAIDs, opiates, intra-articular corticosteroids, and hyaluronic acid derivatives injected into the joint. These agents have not been demonstrated to prevent cartilage loss or slow the loss of joint function.

Given the slow progression of OA, it is anticipated that many humans would need to take an agent for lengthy periods of time. Thus, there is need for therapies that can prevent or reduce the progression of the disease while having a safety profile that allows their use over extended periods of time.

Murine models of OA include those generated by destabilization of the medial meniscus (DMM) or by medial meniscectomy (MM). Approximately 2-6 months after being subjected to DMM or MM, mice are sacrificed and histologic sections of their stifle (knee) joints are stained with toluidine-blue, Safranin-O, and/or hematoxylin and eosin (H&E), revealing the level of cartilage loss (or level of cartilage degeneration, or “OA score”), as well as the degree of osteophyte formation, and the degree of synovial inflammation (termed synovitis).

Alzheimer's Disease (AD).

AD is the most common neurodegenerative disease in humans (Cummings et al., Neurology 51, S2-17; discussion S65-7, 1998). AD affects approximately 10% of people over age 65 and almost 50% of people over age 85. It is estimated that by the year 2025, about 22 million individuals will be afflicted with AD. AD is characterized by a slowly progressive dementia. Definitive diagnosis of AD is made if the triad of dementia, neurofibrillary tangles, and senile plaques (the histologic findings are determined post-mortem). Senile plaques are invariably found in the brains of patients with AD. The principal constituent of senile plaques is amyloid beta protein (Aβ (Iwatsubo et al., Neuron 13:45-53, 1994; Lippa et al., Lancet 352:1117-1118, 1998). Aβ is a 42-amino-acid peptide that is derived from the amyloid precursor protein (APP), which is a transmembrane glycoprotein with a variety of physiologic roles, for instance in cell proliferation, adhesion, cell signaling, and neurite outgrowth (Sinha et al., Ann N Y Acad Sci 920:206-8, 2000). APP is normally cleaved within the Aβ domain, generating a secreted fragment. However, alternative processing leads to cleavage of APP such that it generates soluble Aβ that can accumulate within senile plaques. Currently available drugs for AD are central cholinesterase inhibitors that increase the concentration of postsynaptic acetylcholine in the brain (Farlow and Evans, Neurology 51, S36-44; discussion S65-7, 1998; Hake, Cleve Clin J Med 68, 608-9:613-4, 616, 2001). These drugs provide minimal clinical benefit in only a few cognitive parameters.

Macular Degeneration.

Macular degeneration can be of the wet type, related to retinal neovascularization and vascular leak, but is more commonly of the dry type, also known as age-related macular degeneration (AMD). AMD is a chronic disease associated with loss of central vision, with blurred vision, and ultimately with blindness. Activation of innate immunity, involving complement activation and cytokine production by macrophages and microglia, has been implicated in development of AMD. Anti-inflammatory therapy, including corticosteroids, NSAIDs, methotrexate, rapamycin, and biologic agents (e.g., TNF inhibitors and complement inhibitors) may slow the progression of AMD (Wang et al, 2011. Eye (2011)25, 127-139). However, because these treatments are not curative and AMD is a chronic, non-fatal disease, their use is limited by risk of toxicity.

Chronic Infections Associated with Inflammation

HIV immune activation syndrome. The HIV virus is the cause of AIDS, a disease that is nearly always fatal if left untreated. However, treatment with highly active antiretroviral therapy (HAART) can convert AIDS from a fatal disease to a chronic condition. However, despite lower viral loads and even a reconstituted immune system (as measured by peripheral CD4⁺ T-cell counts), HIV-infected individuals treated with HAART are still at increased risk of morbidity and mortality, primarily as a result of metabolic and cardiovascular problems that arise from chronic immune dysregulation. The cause of the immune activation observed in HIV infection is unknown, but may involve the continued low-grade replication of HIV virus, activation of the endosomal TLR7 receptor, and induction of CD8⁺ T-cell responses (Ipp et al, Clin Chim Acta. 2013; 416:96-9). Additionally, irreversible damage to the immune cells of the gut mucosa results in increased bacterial and endotoxin translocation and thus systemic inflammation (Deeks 2011 Annu Rev Med. 62:141-55). As expected, levels of cytokines (e.g., TNF and IL-6), other inflammatory markers (also termed biomarkers; e.g., C-reactive protein), and coagulation markers (e.g., D-dimer) are still abnormally high in AIDS patients despite successful HAART therapy (Deeks 2011. Annu Rev Med. 62:141-55).

Other chronic infections can also cause persistent inflammation. Such infections include chronic hepatitis B virus infection, chronic hepatitis C virus infection, cytomegalovirus (CMV) infection, herpes simplex virus (HSV) infection, Epstein Barr virus (EBV) infection, chronic pseudomonas infection, chronic Staphlococcus infection, and other chronic viral, bacterial, fungal, parasitic, and other infections.

### Metabolic Inflammatory Diseases

Non-Alcoholic Fatty Liver Disease (NAFLD) and Non-Alcoholic Steatohepatitis (NASH).

NAFLD and non-alcoholic steatohepatitis (NASH) are conditions associated with fatty infiltration of the liver. NAFLD is one cause of a fatty liver, occurring when fat is deposited (steatosis) in the liver not due to excessive alcohol use (Clark J M et al, J. American Medical Association 289 (22): 3000-4, 2003). It can be related to insulin resistance and the metabolic syndrome and may respond to treatments originally developed for other insulin-resistant states (e.g. diabetes mellitus type 2) such as weight loss, metformin and thiazolidinediones.

NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. NASH is a progressive disease: over a 10-year period, up to 20% of patients with NASH will develop cirrhosis of the liver, and 10% will suffer death related to liver disease. NASH is the most extreme form of NAFLD, and is regarded as a major cause of cirrhosis of the liver of unknown cause (McCulough A J et al, Clinics in Liver Disease 8 (3): 521-33, 2004).

Common findings in NAFLD and NASH are elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver disease and can be used to assess the severity of the inflammation and resultant fibrosis (Adams L A et al, Postgrad Med J 82(967):315-22, 2006). Non-invasive diagnostic tests have been developed, such as FibroTest, that estimates liver fibrosis, and SteatoTest, that estimates steatosis, however their use has not been widely adopted (McCulough A J et al, Clinics in Liver Disease 8 (3): 521-33, 2004).

Although fatty infiltration alone does not cause liver damage, when it is accompanied by an inflammatory reaction it can lead to fibrosis and liver cirrhosis and ultimately hepatic failure. The inflammation in NASH is characterized by infiltration of the liver by macrophages and lymphocytes, as well as alterations in the liver's macrophage-like Kupfer cell population (Tilg, et al, 2010. Hepatology. 52(5):1836-46). Inflammatory cytokines, particularly TNF, are central to the pathology of NASH. The source of TNF is unclear: it may be peripheral, i.e., inflammatory adipose tissue, or local, i.e., innate immune cells activated by portal-derived endotoxin or by free fatty acid. The endotoxin-responsive TLR4 receptor has been shown to be critical to disease in a mouse model of NASH (Tsukumo et al, Diabetes 2007. 56(8):1986-98).

A large number of treatments for NAFLD and NASH have been studied. Treatment approaches include: (i) Treatment of nutrition and excessive body weight, (ii) weight loss, (iii) weight loss surgery, (iv) insulin sensitizers including metformin and thiazolidinediones, (v) Vitamin E can improve some symptoms, (vi) statins have been shown to improve liver biochemistry and histology in patients with NAFLD; McCulough A J et al, supra; Chalasani N. et al, Gastroenterology 142(7):1592-1609, 2012).

Type II Diabetes Mellitus and Metabolic Syndrome.

Type II diabetes mellitus is characterized by insulin resistance and hyperglycemia, which in turn can cause retinopathy, nephropathy, neuropathy, or other morbidities. Additionally, diabetes is a well-known risk factor for atherosclerotic cardiovascular disease. Metabolic syndrome refers to a group of factors, including hypertension, obesity, hyperlipidemia, and insulin resistance (manifesting as frank diabetes or high fasting blood glucose or impaired glucose tolerance), that raises the risk of developing heart disease, diabetes, or other health problems; (Grundy et al, Circulation. 2004; 109:433-438). There is a well-characterized progression from normal metabolic status to a state of impaired fasting glucose (IFG: fasting glucose levels greater than 100 mg/dL) or to a state of impaired glucose tolerance (IGT: two-hour glucose levels of 140 to 199 mg/dL after a 75 gram oral glucose challenge). Both IFG and IGT are considered pre-diabetic states, with over 50% of subjects with IFG progressing to frank type II diabetes within, on average, three years (Nichols, Diabetes Care 2007. (2): 228-233). The insulin resistance is caused, at least in part, by chronic low-grade inflammation (Romeo G R et al, Arterioscler Thromb Vasc Biol. 2012 32(8):1771-6; de Luca C et al, FEBS Lett. 2008 582(1):97-105; Ma K et al, Diabetes Metab Res Rev. 2012 28(5):388-94). Macrophages accumulate in obese adipose tissue, where they produce TNF and other inflammatory cytokines in response to stimulation with saturated fatty acids and circulating lipopolysaccharide (LPS) (Johnson et al, Cell 2013. 152(4):673-84; Bhargava P et al, Biochem J. 2012 442(2):253-62). Moreover, TNF inhibition can abrogate insulin resistance (Johnson et al, Cell 2013. 152(4):673-84).

Atherosclerotic Cardiovascular Disease.

Atherosclerosis is a disease of the arterial wall. It is characterized by accumulation of fatty materials in the arterial wall, resulting in development of fatty plaques, which may rupture and cause vascular occlusion and ischemia. If such vascular occlusion and ischemia occur in a coronary artery, myocardial infarction may result. The atherosclerotic lesion comprises a highly inflammatory milieu characterized by the accumulation of inflammatory cells, including macrophages and to a lesser extent T and B cells, and the production of high levels of inflammatory cytokines, chemokines, and MMPs (Libby et al, Nature 2011. 473(7347):3170-25). Atherosclerosis may also be associated with low-grade systemic inflammation, as evidenced by high levels of high-sensitivity CRP (hsCRP) in the blood, an abnormality that can be partially countered by treatment with the drug rosuvastatin (Libby et al, Nature 2011. 473(7347):3170-25).

### Publications

US20070003636A1, entitled “Statins (HMG-COA reductase inhibitors) as a novel type of immunomodulator, immunosuppressor and anti-inflammatory agent”, Mach, Francois; US20100075923A1, entitled “Method of enhancing TGF-beta signaling”, Huang et al.; US20010002401A1 entitled “Treating or preventing the early stages of degeneration of articular cartilage or subchondral bone in mammals using carprofen and derivatives”, Evans et al.; US20080319010A1 entitled “Use of Chloroquine to Treat Metabolic Syndrome”, Kastan et al. NCT00065806-Atherosclerosis Prevention in Pediatric Lupus Erythematosus (APPLE); Parquet et al. (2009) Antimicrobial agents and chemotherapy 53:6; Pareek et al. (2009) Indian Journal of Pharmacology 41(3):125-128; NCT01148043-Pharmacological Treatment In Osteoarthritis (FABIO); Vuolteenaho et al. (2005) Scand J Rheumatol 34:475-479; NCT01645176-Hydroxychloroquine/Atorvastatin in the Treatment of Osteoarthritis (OA) of the Knee; Wu et al. (2007) Medical Hypotheses 69, 557-559; Simopoulou et al. (2010) J Orthop Res 28:110-115. Canadian Patent no. 784,722, Dennis et al., issued May 7, 1968. Munster et al. (2002) Arthritis and Rheumatism 46(6):1460-1469. NCT 01645176.

### SUMMARY OF THE INVENTION

Compositions and methods are provided for preventing or treating the early stages of inflammatory diseases, including autoimmune diseases, degenerative inflammatory diseases, metabolic inflammatory diseases, cancer associated with inflammation, and other inflammatory diseases by administration to an individual of an effective dose of a synergistic combination of active agents comprising or consisting essentially of an aminoquinoline, e.g. HCQ, etc. as defined herein, and a statin, e.g. atorvastatin, etc., as defined herein. Treatment of inflammatory disease at an early time point by the methods of the invention can substantially reduce or prevent disease development, disease progression, or the development of clinical symptoms. In some embodiments treatment is initiated when individuals are at increased risk for development of a disease to prevent development of the disease, to treat early signs or symptoms of disease, or to reverse early signs or symptoms of disease. Individuals who are at increased risk for development of disease may be asymptomatic, may be asymptomatic but have early signs of disease, or may have early symptoms of disease. In other embodiments, treatment is initiated when individuals have early-stage disease to prevent progression of disease, to treat early signs or symptoms of disease, or to reverse early signs or symptoms of disease. When individuals have early-stage disease they have early symptoms or signs of disease, may have intermittent or mild symptoms, or may be asymptomatic and only exhibit signs of disease. In other embodiments, treatment is initiated when individuals have established disease to prevent progression of disease. In individuals with established disease, treatment is initiated to prevent progression of disease, to treat signs and symptoms of disease, or to reverse signs and symptoms of disease. Administration of the combination therapy of the invention may continue for an extended period of time, for example over a period of months or years.

The active agents can be administered separately, or can be co-formulated in a single-unit dose. Each or both of the active agents can be formulated in various ways, including without limitation a solid oral dosage form, for example in a unit dose. An oral dosage form may provide for delayed-release or sustained-release in a controlled manner over at least a 12-hour period.

In some embodiments, a pharmaceutical formulation comprising an effective dose of an aminoquinoline, e.g. HCQ, and a pharmaceutically acceptable excipient is provided, usually in combination with a statin, e.g. atorvastatin. In some embodiments, the formulation comprises or consists essentially of an aminoquinoline and an effective dose of a statin. In some embodiments, a formulation comprises a unit dose of the combination therapy.

In some embodiments, the combination of active agents comprises or consists essentially of HCQ or an equivalent in a daily dose of at least about 25 mg (0.25 mg/kg/day), at least about 50 mg (0.5 mg/kg/day), at least about 100 mg (1.4 mg/kg/day), at least about 155 mg (2 mg/kg/day), at least about 200 mg (2.8 mg/kg/day), at least about 250 mg (3.5 mg/kg/day), at least about 300 mg/day (4.29 mg/kg/day), least about 310 mg (4.42 mg/kg/day), and not more than about 1,200 mg (17.1 mg/kg/day), not more than about 800 mg (11.4 mg/kg/day), not more than about 700 mg (10 mg/kg/day); and atorvastatin or an equivalent in a daily dose of at least about 1 mg (0.014 mg/kg/day), at least about 5 mg (0.07 mm/kg/day), at least about 10 mg (0.14 mg/kg/day), and not more than about 100 mg (1.4 mg/kg/day), not more than about 80 mg (1.14 mg/kg/day). The mg/kg/day dosage through is based on an estimated average body weight of humans of approximately 70 kg (Walpole et al, BMC Public Health (BMC Public Health 2012, 12:439) 12: 439).

In some embodiments a package suitable for use in commerce is provided for treating inflammation according to the methods of the invention, e.g. a pharmaceutical formulation comprising or consisting essentially of aminoquinoline, e.g. HCQ, in combination with a second agent, e.g. a statin; and associated with said carton or container printed instructional and informational material, which may be attached to said carton or to said container enclosed in said carton, or displayed as an integral part of said carton or container, said instructional and informational material stating in words which convey to a reader thereof that the active ingredients, when administered to an individual in the early stages of inflammatory disease, will ameliorate, diminish, actively treat, reverse or prevent any injury, damage or loss of tissue subsequent to early stages of disease. The package comprising carton and container as above-described may conform to all regulatory requirements relating to the sale and use of drugs, including especially instructional and informational material.

At Increased Risk for Development of an Inflammatory Disease.

In some embodiments the methods of the invention comprise the step of identifying individuals “at-risk” for development of, or with “early-stages” of, an inflammatory disease. “At risk” for development of an inflammatory disease includes: (1) individuals whom are at increased risk for development of an inflammatory disease, and (2) individuals exhibiting a “pre-clinical” disease state, but do not meet the diagnostic criteria for the inflammatory disease (and thus do not have the inflammatory disease).

Individuals “at increased risk” for development (also termed “at-risk” for development) of an inflammatory disease can be identified based on their exhibiting or possessing one or more of the following: family history of disease; the presence of certain genetic variants (genes) or combinations of genetic variants; the presence of certain physical findings, laboratory test results, imaging findings, or biomarker test results associated with development of the inflammatory disease; the presence of clinical signs related to the inflammatory disease; the presence of certain symptoms related to the inflammatory disease (although the individual is frequently asymptomatic); the presence of markers (also termed “biomarkers”) of inflammation; and other findings that indicate an individual has an increased likelihood over the course of their lifetime to develop an inflammatory disease. Most individuals at increased risk for development of an inflammatory disease are asymptomatic, and are not experiencing any symptoms related to the disease that they are at an increased risk for developing.

Included, without limitation, in the group of individuals at increased risk of developing an inflammatory disease, are individuals exhibiting “pre-clinical disease state”. The pre-disease state is diagnosed based on developing symptoms, physical findings, laboratory test results, imaging result, and other findings that result in their meeting the diagnostic criteria for the inflammatory disease, and thus being formally diagnosed. Individuals with “pre-clinical disease” exhibit or possess findings that suggest an individual is in the process of developing the inflammatory disease, but do not have findings, including the symptoms, clinical findings, laboratory findings, and/or imaging findings, etc. that are necessary to meet the diagnostic criteria for a formal diagnosis of the inflammatory disease. In some embodiments, individuals exhibiting a pre-clinical disease state possess a genetic variant or a combination of genetic variants that place them at increased risk for development of disease. In some embodiments, these individuals have laboratory results, or physical findings, or symptoms, or imaging findings that place them at increased risk for development of an inflammatory disease. In some embodiments, individuals with preclinical disease states are asymptomatic. In some embodiments, individuals with pre-clinical disease states exhibit increased or decreased levels of the expression of certain genes, expression of certain proteins, metabolic markers, and other markers.

In some embodiments, individuals at increased risk for an inflammatory disease exhibit increased inflammatory markers (also termed “inflammatory biomarkers”), including c-reactive protein (CRP), high-sensitivity CRP (hs-CRP), erythrocyte sedimentation rate (ESR), cytokines in blood or other biological fluids, and other markers of inflammation. Method can include determining the presence of inflammation prior to treatment, for example by detection and analysis of one or more biomarker(s) associated with inflammation, where an individual in an early stage of disease showing signs of inflammation is selected for treatment with a formulation of the invention. In some embodiments the treatment ameliorates, diminishes, actively treats, reverses or prevents tissue injury. In some embodiments the inflammatory disease is an autoimmune disease, for example RA, multiple sclerosis, systemic lupus erythematosus, Sjogren's Syndrome, etc. In some embodiments the disease comprises an inflammatory component contributing to a metabolic disease, for example metabolic syndrome, type II diabetes, insulin resistance, atherosclerosis, etc. In some embodiments the disease is a degenerative disease such as OA, Alzheimer's disease, or macular degeneration.

In some embodiments the methods of the invention comprise the step of identifying individuals at increased risk for development of an inflammatory disease. These individuals at increased risk for development of an inflammatory disease can have risk factors for disease and/or be in a “pre-clinical” state, and are sometimes asymptomatic. Treatment at this point is exceptionally valuable in preventing development of the inflammatory disease; however, it is important to prescribe a safe and efficacious therapy that can be tolerated over long periods of time, as is provided by the present invention.

Early-Stage Inflammatory Disease.

The determination of “early-stage disease” in an individual can comprise analyzing the individual for the presence of at least one marker indicative of the presence of early disease. In some embodiments the method comprises analyzing an individual for the presence of one, two, three, four, or more markers that are diagnostic for early disease. In some embodiments at least one of the marker(s) is an imaging marker, including without limitation: arthroscopy, radiographic imaging, ultrasound imaging, magnetic resonance imaging (MRI), computed tomography (CT), etc. In some embodiments at least one of the marker(s) is a molecular marker, where a biological sample is obtained from the individual and analyzed for the presence of a molecule, e.g. a cytokine, antibody, cartilage component, protease, etc. and compared to a control or reference value, wherein altered level of the molecular marker is indicative of early disease. In some embodiments, early-stage disease is defined by the presence of symptoms for less than 6 months. In some embodiments, early-stage disease is defined by being formally diagnosed with the inflammatory disease for less than 6 months. In some embodiments, early-stage disease is associated with no symptoms. In some embodiments, early-stage disease is associated with mild symptoms. In some embodiments, early-stage disease is associated with intermittent symptoms, such as symptoms occurring only once every couple years, or symptoms occurring once every couple months, or symptoms occurring once every couple days, or symptoms occurring for only part of each day.

Determination of inflammation in an individual with early-stage disease can comprise analyzing the individual for the presence of at least one marker indicative of the presence of inflammation. In some embodiments the method comprises analyzing an individual for the presence of one, two, three, four, or more markers that are diagnostic for inflammation, which can be systemic or localized inflammation. In some embodiments at least one of the marker(s) is an imaging marker, including without limitation radiographic imaging, ultrasound imaging, magnetic resonance imaging (MRI), computed tomography (CT), etc. In some embodiments at least one of the marker(s) is a molecular marker, where a biological sample is obtained from the individual and analyzed for the presence of a molecule, e.g. a cytokine, antibody, cartilage component, protease, etc. and compared to a control or reference value, wherein altered level of the molecular marker is indicative of inflammation. In some embodiments the marker indicative of inflammation indicates the presence of local inflammation, i.e. inflammation present at the affected joint.

Established Inflammatory Disease.

In certain embodiments, this invention is for the treatment of individuals with established inflammatory disease. The inflammatory disease is diagnosed based on an individual exhibiting symptoms, signs, clinical features, laboratory test results, imaging test results, biomarker results, and other findings that enable a physician to formally diagnose that individual with the inflammatory disease. In some embodiment, established inflammatory disease is an inflammatory disease for which an individual has had a formal diagnosis of the disease made by a physician for longer than 6 months. In established inflammatory disease, the signs or symptoms of disease may be more severe. In established inflammatory disease, the disease process may cause tissue or organ damage. As described above, in certain embodiments determination of inflammation in an individual with established disease can comprise analyzing the individual for the presence of at least one marker indicative of the presence of inflammation.

### BRIEF DESCRIPTION OF THE DRAWINGS

FIG. 1. Treatment with the combination of hydroxychloroquine and atorvastatin inhibited the development of and reduced the severity of osteoarthritis (OA) in a mouse model. C57BL6 (B6) mice (n=8 per group) were surgically induced to develop OA by surgical destabilization of the medial meniscus (DMM). One week after DMM, treatment was initiated with hydroxychloroquine sulfate (HCQ) 100 mg/kg/day and atorvastatin calcium (Atorva) 40 mg/kg/day delivered by oral gavage once per day. One week after DMM, mice run and walk normally without a limp or any signs of pain, but several months later they develop fully symptomatic OA, which manifests as gait abnormalities (as described in Nat. Med. 2011 Nov. 6; 17(12):1674-9. doi: 10.1038/nm.2543. PMID: 22057346). Thus, although the mice are asymptomatic one week after undergoing DMM, they are in the pre-clinical or early stages of OA. In this experiment, mice surgically induced to develop OA with the DMM procedure were treated with vehicle (A), 100 mg/kg/d of hydroxychloroquine sulfate (HCQ) alone (B), 40 mg/kg/d of atorvastatin calcium (Atorva) alone (C), or the combination of hydroxychloroquine sulfate and atorvastatin calcium (HCQ+Atorva) (D). After 3 months of treatment following DMM, the mice were sacrificed, their stifle joints harvested and fixed, and joint sections stained with safranin-O. Images of representative safranin-O stained sections of the medial region of the stifle joints are presented. Arrowheads indicate areas of cartilage degeneration. Quantitative scoring of the results from all mice is presented in FIG. 2.

FIG. 2. Treatment with the combination of hydroxychloroquine and atorvastatin prevented the development of and reduced the severity of osteoarthritis (OA) in a mouse model. As part of the same experiment described in FIG. 1, C57BL6 (B6) mice (n=8 per group) were surgically induced to develop OA by destabilization of the medial meniscus (DMM). One week after DMM, a time point at which the surgically induced mice are asymptomatic or have mild pre-OA joint symptoms, treatment was initiated with hydroxychloroquine sulfate (HCQ) 100 mg/kg/day and/or atorvastatin calcium 40 mg/kg/day delivered by oral gavage once per day. After 3 months, mice were sacrificed, their stifle joints harvested, joint sections cut, and tissue sections stained with safranin-O. An examiner blinded to treatment used microscopy to score the severity of OA. The “Cartilage Degeneration Score” (also termed “Cartilage degeneration” or “Histologic score” or “OA Score”) was determined, and represents the severity of cartilage degeneration. Individually, HCQ and atorvastatin each modestly reduced cartilage degeneration, but this reduction was not statistically significant. However, the combination of HCQ and atorvastatin significantly reduced the development of and reduced the severity of OA. Graphs present the mean and the standard error of the mean (s.e.m.), *P<0.05; **<0.01; *** <0.001 by t test. NS=non-significant.

FIG. 3. Treatment with the combination of hydroxychloroquine (HCQ) and atorvastatin (Atorva) attenuated synovitis and osteophyte formation in a mouse model of OA. As part of the same experiment described in FIGS. 1 and 2, histologic sections from the operated knees from the mice described in FIG. 2 were stained with H&E or safranin-O, and the degree of synovial inflammation (synovitis) or osteophyte formation was quantitated by an examiner blinded to treatment group. Scores for osteophyte formation and synovitis were recorded for the femoral-medial and the tibial-medial condyles on the operated side of the joint, and the scores for the two regions were summed. (A) Osteophyte and synovitis scores in mice treated with vehicle, HCQ, atorvastatin, or a combination of HCQ and atorvastatin. Scores were compared between each treatment group and the control (vehicle-treated) group by t test. The combination of hydroxychloroquine and atorvastatin significantly reduced both the level of synovitis and the formation of osteophytes to a significantly greater extent than did treatment with either atorvastatin alone or HCQ alone (B), whereas treatment with either agent alone was not as potent (A, B). Calculation of the “Osteophyte score” and “Synovitis score” is described in detail in Wang et al. (Nat. Med. 2011 Nov. 6; 17(12):1674-9. doi: 10.1038/nm.2543. PMID: 22057346).

FIG. 4. Treatment with the combination of hydroxychloroquine and atorvastatin reduced joint inflammation in humans with medial-compartment knee osteoarthritis (OA) in a 16-week open-label clinical trial. We performed a 16-week, open-label clinical trial in humans with OA involving the medial compartment of the knee and evidence of ongoing active synovitis on gadolinium-enhanced MRI scan, to determine the ability of treatment with the combination of hydroxychloroquine and atorvastatin to reduce the “MRI Synovitis Score” in the index knee. This trial was registered with and assigned the ClinicalTrials.gov Identifier: NCT01645176. The MRI Synovitis Score was determined both at baseline and after 16 weeks of treatment, and only subjects with a MRI Synovitis Score greater than 7 were enrolled. 6 subjects were enrolled and completed 16 weeks of treated with the combination of hydroxychloroquine sulfate 600 mg and atorvastatin calcium 40 mg by mouth each day for 16 weeks. The MRI Synovitis Scores were analyzed by two-way paired t test, which demonstrated that treatment with the combination of hydroxychloroquine and atorvastatin significantly reduced the amount of synovitis (P=0.024). We believe that the reduction in synovitis observed will translate to the combination of hydroxychloroquine and atorvastatin preventing the development of and reducing the progression of OA in humans.

FIG. 5. A combination of hydroxychloroquine and atorvastatin reduced the WOMAC Pain Score, WOMAC Function Score, and WOMAC Combined Score in humans with medial-compartment knee osteoarthritis (OA) in a 16-week, open-label clinical trial. In the medial-compartment knee OA trial described in FIG. 4, we also measured Western Ontario and McMaster Universities Arthritis Index (WOMAC) Pain (A), Function (B), and Combined (C) Scores (see McConnell et al., The Western Ontario and McMaster Universities Osteoarthritis Index (WOMAC): a review of its utility and measurement properties. Arthritis Rheum 2001; 45: 453-61). The WOMAC Pain, Function, and Combined scores were analyzed by one-tailed paired t tests, which demonstrated that treatment with the combination of hydroxychloroquine and atorvastatin for 16 weeks significantly reduced the WOMAC Pain Score (P=0.035), WOMAC Function Score (P=0.005), and WOMAC Combined Score (P=0.003).

FIG. 6. Treatment with a combination of hydroxychloroquine and atorvastatin prevented the development of and reduced the severity of rheumatoid arthritis (RA) in a mouse model. DBA/1 mice (n=15 per group) were induced to develop collagen-induced arthritis (CIA), a mouse model for RA, by immunization with type II collagen emulsified in complete Freund's adjuvant (CFA) followed 21 days later by boosting with type II collagen emulsified in incomplete Freund's adjuvant (IFA). Following the first immunization (with type II collage in CFA), a time at which mice had no overt symptoms of RA but had been induced to develop RA, treatment was initiated with hydroxychloroquine sulfate (HCQ) 50 mg/kg/day by oral gavage for 2 weeks, then increased to a loading dose of 100 mg/kg/day by oral gavage to efficiently achieve therapeutic drug concentrations in the tissues, and then at day 21 the dose was reduced back to and continued at a lower maintenance dose of 50 mg/kg/d by oral gavage. At day 21, a time at which the mice are asymptomatic but are in a “pre-RA” state and exhibit increased inflammation (owing to the immunization with CFA), treatment with atorvastatin calcium 1 mg/kg/day was initiated. Individual mice started to developed clinical arthritis (overt swelling of the joints) on approximately day 28. The Visual Scoring System (described in Paniagua et al, Journal Clinical Investigation, 2006, 116:2633-2642.) was used for evaluating the severity of arthritis at serial timepoints after administration of the boost immunization, and the mean and standard error of the mean (s.e.m.) are displayed. Mann Whitney U test comparisons between the individual treatment groups demonstrated that treatment with hydroxychloroquine and atorvastatin prevented the development of arthritis and significantly reduced disease activity as compared to treatment with hydroxychloroquine alone, atorvastatin alone, or vehicle control.

FIG. 7. A combination of hydroxychloroquine and atorvastatin reduced the development and severity of multiple sclerosis (MS) in a mouse model. Experimental autoimmune encephalomyelitis (EAE), a mouse model of MS, was induced in SJL mice (n=10 per group) by immunization with proteolipid protein peptide 139-151 (PLP 139-151) in complete Freund's adjuvant (CFA). Within 10 days of being immunized with PLP 139-151, SJL mice are inflamed and develop autoantibodies, a state reflecting preclinical or early-stage MS (Nat. Biotechnol. 2003 September; 21(9):1033-9. Epub 2003 PMID: 12910246). After immunization, mice were treated with a loading dose of hydroxychloroquine sulfate (HCQ) 100 mg/kg/day (in order to more efficiently achieve therapeutic concentrations of HCQ in the tissues) in combination with atorvastatin calcium 1 mg/kg/day, and 8 days after immunization the dose of HCQ sulfate was lowered to 50 mg/kg/day (the maintenance dose) given in combination with atorvastatin calcium 1 mg/kg/day. For the atorvastatin alone (1 mg/kg/d) and the vehicle control groups, treatment was initiated following immunization. Mice were scored daily for the severity of EAE as previously described (Robinson et al, Nature Biotechnology, 2003, 21(9):1033-1039), and the mean clinical scores with the standard error of the mean (s.e.m.) are displayed. Mann Whitney U test comparisons between the groups demonstrated that EAE was prevented in and significantly less severe in the group treated with the combination of hydroxychloroquine and atorvastatin (HCQ & Ator) than in the groups treated with HCQ alone, atorvastatin alone, or the vehicle control.

FIG. 8. A combination of hydroxychloroquine and atorvastatin prevents development of and reduces the levels of inflammation-related metabolic and tissue injury biomarkers in a mouse model of diet-induced obesity (DIO). For assessing the effect of combination therapy with hydroxychloroquine plus atorvastatin on a mouse model of hyperlipidemia, type II diabetes, and non-alcoholic fatty liver disease (NAFLD), C57BL/6 mice (n=5 on average per group) were fed a high-fat “western-style” diet (Taconic) for 6 weeks. The mice exhibited normal behavior and no overt symptoms throughout this time, but developed a pre- or early-disease state as evidence by elevations in blood glucose, cholesterol, triglycerides. After initiation of the high-fat diet, these asymptomatic pre-disease mice were treated with the combination of hydroxychloroquine sulfate (HCQ; 100 mg/kg/day) plus atorvastatin calcium (Atorv; 40 mg/kg/day), or with vehicle. After 4 weeks of treatment, non-fasting sera were analyzed. The levels of inflammation-related metabolic biomarkers were compared between the mice treated with vehicle control and the mice treated with the combination of HCQ and atorvastatin by using a two-tailed t test. Levels of total cholesterol, triglycerides, and LDL cholesterol were significantly lower in the mice treated with the combination of HCQ and atorvastatin than in the mice treated with vehicle. In addition, levels of glucose, a marker of early insulin resistance and early onset of type II diabetes, also were significantly lower in mice treated with the combination of HCQ and atorvastatin than in mice treated with vehicle, as were levels of levels of ALT, a measure of early non-alcoholic steatohepatitis (NASH) (* P<0.05, ** P<0.01). These data demonstrate that treatment with a combination of HCQ plus atorvastatin can attenuate early insulin resistance. They also demonstrate that treatment with a combination of HCQ plus atorvastatin can treat hypercholesterolemia, and thus inhibit the development of atherosclerosis. Further, they demonstrate that treatment with a combination of HCQ plus atorvastatin can treat early NAFLD that leads to the development of NASH. Together, these data show that treatment with a combination of HCQ plus atorvastatin treats early metabolic syndrome and prevents development of metabolic abnormalities and liver injury.

FIG. 9. The combination of hydroxychloroquine and atorvastatin prevented the development of fatty liver and liver injury in a mouse model of diet-induced obesity (DIO). From the experiment in FIG. 8, following 6 weeks of high-fat diet and dosing with the combination of HCQ and atorvastatin mice were sacrificed, and their livers harvested. (A) Livers were formalin-fixed, paraffin-embedded, sectioned and stained with hematoxylin and eosin (H&E). (B) Liver histology was examined under a light microscope and then graded according to the magnitude of steatosis, inflammation, and ballooning degeneration of hepatocytes as based on an established scoring system (Brunt et al, American Journal of Gastroenterology, 94(9):2467-2474, 1999). Briefly, the degree of steatosis was graded 0-4 based on the average percent of fat accumulated hepatocytes per field 200× under H&E staining (grading: 0=<5%, 1=5-25%, 2=26-50%, 3=51-75%, 4=>75%). Inflammation was evaluated by the number of inflammatory cells counted in 10 random fields at 200× magnification. The mean of these numbers was calculated and represents the number of inflammatory cells/mm². Hepatocellular ballooning degeneration was evaluated as either negative (absent=0), positive (present=1), or dominant (present and dominant=2). ***P<0.001 by t test.

FIG. 10. Increased expression of genes encoding cytokines, chemokines, complement components, and other inflammatory mediators in synovial membrane tissue derived from humans with early- or end-stage knee osteoarthritis (OA). Publicly available gene-expression profiles of synovial membrane derived from OA patients and healthy controls (accession # GSE12021) were downloaded from the NCBI's Gene Expression Omnibus (GEO). The results for expression of genes encoding inflammatory proteins, including cytokines, chemokines, complement components, and other mediators, were extracted and subjected to hierarchical clustering. Genes whose expression is higher in individuals with early- or end-stage OA than in healthy controls are displayed in a heatmap. The change in gene expression relative to healthy controls is indicated.

FIG. 11. A Luminex™ System was used for profiling of cytokines in sera and synovial fluid samples provided by the Stanford Arthritis Center, using protocols that include addition of Heteroblock™ (Omega) to prevent heterophilic activity of rheumatoid factor. Levels of multiple cytokines were higher in OA synovial fluids, RA sera, and RA synovial fluids than in healthy sera (A). The Significance Analysis of Microarrays (SAM) statistical algorithm identified cytokines whose levels were higher in OA sera than in healthy sera (false discovery rate <0.05), and the results were subjected to unsupervised clustering and displayed as a heatmap (B).

FIG. 12. The combination of hydroxychloroquine and atorvastatin synergistically reduced anti-CD3/CD28-induced IFN-gamma production by T cells. Mice with CIA were sacrificed, and splenic T cells were isolated with a MACS system and negative selection. Isolated T cells were stimulated from anti-CD3+CD28 dynabeads in the presence of 0 or 0.1 μM HCQ and/or 0, 0.1, 1, or 10 μM atorvastatin for 48 hours, after which culture supernatants were collected and IFN-gamma measured by ELISA. Mean IFN-gamma levels with standard error of the mean values of triplicates are displayed. A combination of 1 μM of hydroxychloroquine (HCQ) and 3 μM of atorvastatin (Ator.) synergistically reduced anti-CD3+CD28-induced stimulation of IFN-gamma production. *P<0.05; **P<0.01; ***P<0.001 by Tukey test.

FIG. 13. The combination of hydroxychloroquine and atorvastatin synergistically reduced proteolipid protein (PLP mediated stimulation splenic cells isolated from mice with experimental autoimmune encephalomyelitis (EAE). Mice with EAE were sacrificed, and splenic cells isolated. Isolated splenic cells were stimulated with proteolipid protein (PLP) in the presence of 0 or 0.1 μM HCQ and/or 0, 0.1, or 10 μM atorvastatin and/or the presence of 0, 1, 3 or 10 μM hydroxychloroquine for 48 hours, following which culture supernatants were collected and IFN-gamma and IL-17 measured by ELISA. Mean IL-17 (A) and IFN-gamma (B) levels with standard error of the mean are displayed. The Tukey test was used to statistically compare results between groups, and demonstrated that the combination of hydroxychloroquine 1 μM and atorvastatin 3 μM synergistically inhibited PLP-induced production of pro-inflammatory IFN-gamma and IL-17. *P<0.05; **P<0.01 by unpaired T test.

FIG. 14. Treatment with the combination of hydroxychloroquine and atorvastatin synergistically reduced cytokine production in synovium derived from joints subjected to DMM to induce development of OA. 20 week old B6 male mice (n=7-10 per treatment arm) were induced to generate destabilization of the medial meniscus (DMM) model. The mice were dosed with vehicle alone, HCQ sulfate (100 mg/kg/day), atorvastatin calcium (40 mg/kg/day), or HCQ sulfate+Atorvastatin calcium orally starting the day after surgical induction of OA. 3 months after surgery, the mice were sacrificed and the synovium of the operated joint isolated. The collected synovial membrane tissues were used to generate protein lysates, on which multiplex cytokine analysis was performed using the Luminex System and the Bio-Rad BioPlex 23plex mouse cytokine detection kit. Each sample was run in triplicate. Only the results for cytokines exhibiting significant differences in their levels by t test (P<0.05) in synovial tissue derived from HCQ+atorvastatin treated mice as compared to HCQ alone or atorvastatin alone treated mice are displayed. (A) Heatmap presenting the relative levels (as compared to mean levels in the vehicle-control treated group). (B) Bar-graph display of mean cytokine levels for the indicated treatment groups. T tests were used to compare the levels between the HCQ+atorvastatin group vs. each of the other groups (vehicle control, HCQ alone, atorvastatin alone), and the results of each comparison are indicated with *P<0.05; ** P<0.01; *** P<0.001. Treatment with the combination of HCQ+atorvastatin, as compared to treatment with HCQ alone or atorvastatin alone, synergistically reduced the levels of multiple synovial tissue cytokines in the stifle (knee) joint surgically induced to develop OA.

FIG. 15. Treatment with the combination of hydroxychloroquine and atorvastatin synergistically reduced MMP3 mRNA expression in murine synovial tissue stimulated with IL-1β 5 ng/ml for 24 hours. Treatment with the combination of HCQ+atorvastatin, as compared to treatment with HCQ alone or atorvastatin alone, synergistically reduced the levels MMP3 mRNA expression in the synovial tissue stimulated with IL-1β, an OA-related cytokine.

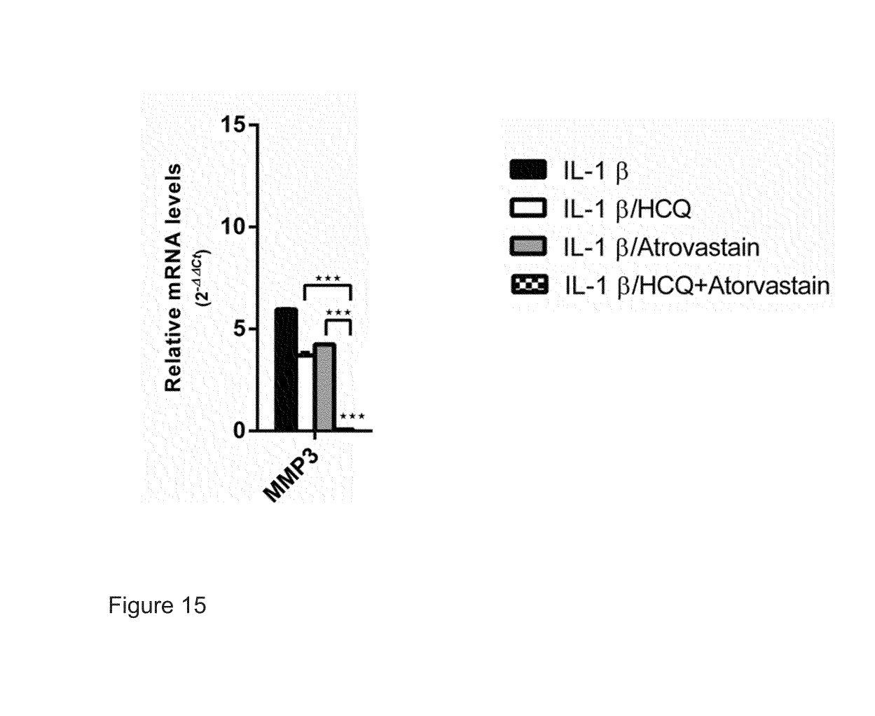

FIG. 16. The combination of hydroxychloroquine and atorvastatin reduces IL-1β-mediated activation of downstream cellular signaling pathways in murine synovial tissue. Murine synovial tissue was isolated and stimulated in vitro with IL-1β5 ng/ml for 24 hours, following which protein lysates were generated and analyzed with a Luminex-based multiplex analysis of phosphorylated (activated) signaling proteins. Treatment with the combination of HCQ+atorvastatin, as compared to treatment with vehicle control, HCQ alone or atorvastatin alone, reduced the levels of activation of multiple cellular signaling pathways.

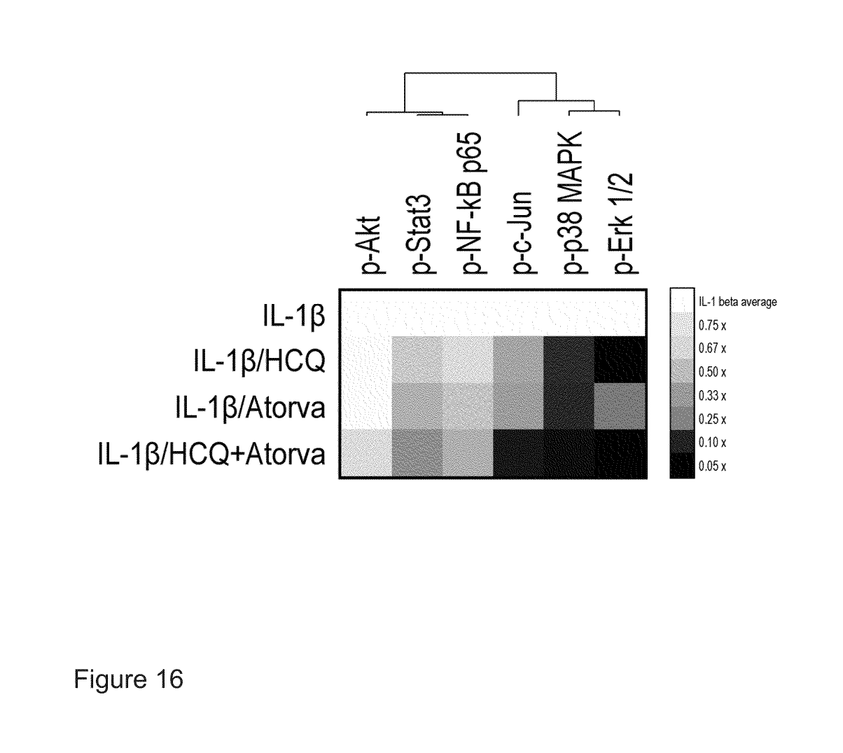

FIG. 17. Examples of aminoquinolines. Aminoquinolines are derivatives of quinoline, most notable for their use as antimalarial drugs. Examples of aminoquinolines are presented.

FIG. 18. Metabolites of hydroxychloroquine. The metabolites of hydroxychloroquine (HCQ) comprise desethylhydroxychloroquine (DHCQ), desethylchloroquine (DCQ), and bisdesethylchloroquine (BDCQ).

FIG. 19. Examples of statins. Examples of statins including atorvastatin, cerivastatin, fluvastatin, lovastatin, mevastatin and pitavastatin are presented.

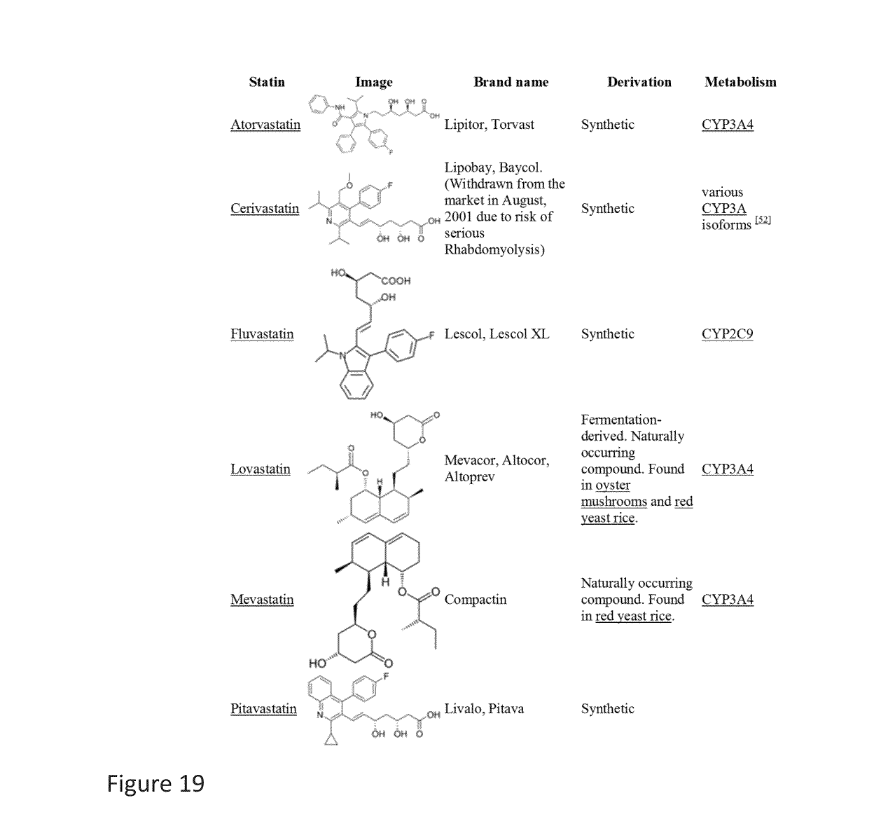

FIG. 20. The combination of atorvastatin and hydroxychloroquine, and the combination of atorvastatin and desethylhydroxychloroquine, attenuated osteoarthritis (OA) in a mouse model. C57BL6 (B6) mice (n=7-10 per group) were surgically induced to develop OA by destabilization of the medial meniscus (DMM). One week after DMM, a time at which the mice were asymptomatic or had mild pre-OA joint symptoms, treatment was initiated with one or more of the following compounds: atorvastatin calcium (Atorva) 40 mg/kg/day, hydroxychloroquine sulfate (HCQ) 100 mg/kg/day, desethylhydroxychloroquine (DHCQ) 100 mg/kg/day—groups were treated by oral gavage once per day with vehicle control, Atorva alone, HCQ alone, the combination of Atorva and HCQ, or the combination of Atorva and DHCQ. After 3 months, mice were sacrificed, their stifle joints harvested, joint sections cut, and tissue sections stained with safranin-O. The mean “Cartilage degeneration scores” in safranin-O-stained sections of the medial region of the stifle joints are presented in the graph. Two-tailed t tests were used for comparing the Cartilage Degeneration Scores between each treatment group and the control (vehicle-treated) group.

FIG. 21. The combination of atorvastatin and hydroxychloroquine, and of atorvastatin and desethylhydroxychloroquine (DHCQ), prevented the development of OA and reduced the severity of cartilage degeneration, osteophyte formation, and synovitis in a mouse model of OA. From the mouse OA experiment presented in FIG. 20, the mean “Cartilage degeneration scores” in safranin-O stained sections of the medial region of stifle joints of mice subjected to DMM were compared between the vehicle-treated group and each of the other treatment groups by two-tailed t tests. (A) The combinations of atorvastatin and hydroxychloroquine, as well as atorvastatin and desethylhydroxychloroquine, both statistically reduced synovitis (inflammation) in the joint (P<0.01), prevented the development of OA based on the cartilage degeneration score (P<0.01), and reduced the severity of OA (P<0.01) as compared to vehicle-treated mice. (B) The mean scores for cartilage degeneration, osteophyte formation, and synovitis in safranin-O stained sections of the medial region of stifle joints of mice subjected to DMM were compared between the single-treatment groups (HCQ alone, or atorvastatin alone) and the combination-treatment groups (HCQ+atorvastatin, or DHCQ+atorvastatin) by two-tailed t tests. Cartilage degeneration, osteophyte formation, and synovitis were all lower in mice treated with either the combination of atorvastatin and hydroxychloroquine (HCQ+Atorva) or the combination of atorvastatin and desethylhydroxychloroquine (DHCQ+Atorva) than in mice treated with HCQ alone or Atorva alone.

FIG. 22. Atorvastatin protects against HCQ-mediated retinal toxicity. Assessment of retinal toxicity in the mouse OA experiment presented in FIG. 20 revealed that treatment of mice with HCQ resulted in retinal toxicity as manifested by histologic abnormalities including nuclear shrinkage, while in contrast treatment with a combination of hydroxychloroquine and atorvastatin did not induce retinal toxicity. At the time of termination as described in FIG. 20, the eyes were carefully microdissected, and fixed in formalin. The fixed eyes were then sectioned to allow visualization of the retina. The retinal cell layer was stained with hematoxylin and eosin (H&E) and evaluated, by using the histologic and quantitative pathology methodology adapted from Shichiri, et al (Shichiri, et al, JBC. 2012 287(4):2926-34. PMID 22147702), for number of nuclei in the retinal ganglion cell layer (GCL), as well as nuclear shrinkage in the GCL, which is suggestive of a selective loss of retinal ganglion cells. Derangement with nuclear shrinkage in the GCL was detected in the retina of mice treated with HCQ alone (see arrow) (B), but not in the retina of mice treated with the combination of HCQ and atorvastatin (C) or atorvastatin alone (D) or vehicle alone (A). Representative images of H&E-stained retinal sections are presented from each treatment group.

FIG. 23. Atorvastatin protects against HCQ-mediated retinal ganglion cell loss and death. At the time of termination as described in FIG. 20, the eyes were carefully microdissected, and fixed in formalin. The fixed eyes were then sectioned to allow visualization of the retina. The retinal cell layer was stained with hematoxylin and eosin (H&E) and evaluated, by using the histologic and quantitative pathology methodology adapted from Shichiri, et al. (Shichiri, et al, JBC. 2012 287(4):2926-34. PMID 22147702), for number of nuclei in the retinal ganglion cell layer (GCL) to measure selective loss and death of retinal ganglion cells. The graph presents quantitation of the number of nuclei in the GCL in the retina of mice treated with different compounds, with the number of nuclei representing the number of viable retinal ganglion cells. The number of viable cells in the GCL for each treatment group was compared with the number of viable cells in vehicle-treated control by two-tailed t test (*P4.05; N.S.=non-significant). The number of cells in the retinal GCL was significantly lower in mice treated with HCQ alone (P≦0.05) as compared to control mice treated with the vehicle control. Treatment with the combination of atorvastatin and HCQ resulted in significantly higher retinal ganglion cell viability (P≦0.05). The number of cells in the GCL did not differ between mice treated with vehicle and mice treated with the combination of HCQ and atorvastatin, or treated with atorvastatin alone. This result, together with the morphologic characteristics observed in FIG. 22, demonstrate that: (1) treatment with HCQ alone induced retinal ganglion cell toxicity and death, (2) treatment with a combination of HCQ and atorvastatin did not result in retinal ganglion cell loss. These data demonstrate that HCQ mediates retinal toxicity, and that co-administration of atorvastatin protects against HCQ-mediated retinal toxicity.

### DETAILED DESCRIPTION OF THE EMBODIMENTS

Compositions and methods are provided for preventing or treating the early stages of inflammatory diseases, which may be pre-symptomatic, including autoimmune diseases, degenerative inflammatory diseases, metabolic inflammatory diseases, cancer associated with inflammation, and other inflammatory diseases by administration to an individual of an effective dose of a combination of aminoquinoline and statin. In some embodiments the compositions are utilized to treat low-grade inflammation associated with disease, including but not limited to (i) OA, with the purpose of preventing any of the following: cartilage destruction, pain, and/or loss of joint function; (ii) RA, with the purpose of preventing any of the following: pain, swelling, stiffness, joint-space narrowing, bone erosion, or joint instability or destruction; (iii) MS, with the purpose of preventing any of the following: numbness, weakness, pain, dizziness, visual complications, bowel or bladder dysfunction, or systemic symptoms, including but not limited to fatigue, fevers, chills; (iv) type II diabetes, with the purpose of preventing the development of symptoms including increased thirst, increased urination, and fatigue; (v) nonalcoholoic steatohepatitis (NSA), including the treatment of nonalcoholic fatty liver disease (NAFLD) to prevent the development of NASH and its sympoms including fatigue and weight loss; and (vi) other inflammatory disorders for which attenuation or reduction of inflammation can prevent the onset of clinical symptoms and/or clinical or subclinical organ or tissue damage.

All publications, patents, and patent applications mentioned in this specification are herein incorporated by reference to the same extent as if each individual publication, patent, or patent application was specifically and individually indicated to be incorporated by reference.

Inflammatory Disease.

Inflammatory diseases are diseases that involve inflammation. The presence of inflammation can be detected by a variety of approaches, including clinical history, physical examination, laboratory testing, histologic analysis of tissue, analysis of biomarkers, and imaging. Clinical features and physical exam features of inflammation include swelling, effusions, edema, redness, warmth, pain, or associated pathologically with the influx of inflammatory cells or production of inflammatory mediators. Laboratory testing and/or histologic analysis can demonstrate increased numbers of inflammatory cells. Imaging can reveal findings including enhancement of tissues, edema and swelling of tissues, and other findings indicative of inflammation.

Low-grade inflammation.

The presence of low-grade inflammation is characterized by a elevations in the local or systemic concentrations of cytokines such as TNF-α, IL-6, and c-reactive protein (CRP), and occurs in adiposity, osteoarthritis, Alzheimer's disease, type II diabetes, metabolic syndrome, coronary artery disease, nonalcoholic fatty liver disease (NAFLD) and many chronic and degenerative diseases. Low-grade inflammation is manifest by inflammation present at a level below the “high-grade” inflammation detected in active autoimmune diseases (such as active rheumatoid arthritis, psoriasis, Crohn's disease, systemic lupus erythematous and other autoimmune states) and in certain viral and bacterial infections during which humans experience clinical symptoms (such as influenza virus infection, Staphylococcus aureus infection, and other infections).

Amelioration of inflammation. The reduction of inflammation as indicated by dissipation of inflammation, a reduction in number of inflammatory cells or in levels of inflammatory mediators as evidenced by symptomatic relief (including but not limited to pain relief), radiographic changes, biochemical changes, pathologic/histologic changes, decreased progression of such markers of inflammation, decreased development of findings indicative of tissue or organ damage, decreased development of symptoms or signs of disease, or decreased development of disease.

Symptoms of disease. A symptom is a departure from normal function or feeling which is noticed by an individual, indicating the presence of disease or abnormality. A symptom is subjective, observed by the individual patient, and cannot be measured directly.

Signs of disease. A sign of disease or medical sign is an objective indication of some medical fact or characteristic that may be detected during a physical examination, by an in vivo examination of a patient, by a laboratory test, by a radiographic or other imaging test, or by another. Signs may have no meaning to the patient, and may even go unnoticed, but may be meaningful and significant to the healthcare provider in assisting the diagnosis of medical condition(s) responsible for the patient's symptoms. Examples of signs include elevated blood pressure, elevated cholesterol, a clubbing of the fingers (which may be a sign of lung disease, or many other things), arcus senilis, loss of proteoglycans in the cartilage, increased blood glucose, increased liver function tests, and other findings. Signs are any indication of a medical condition that can be objectively observed (i.e., by someone other than the patient), whereas a symptom is merely any manifestation of a condition that is apparent to the patient (i.e., something consciously affecting the patient). From this definition, it can be said that an asymptomatic patient is uninhibited by a disease. However, a doctor may discover the sign hypertension in an asymptomatic patient, who does not experience “disease”, and the sign indicates a pre-clinical or early-stage disease state that poses a hazard to the patient.

Administration of agents. Administration of a drug or other chemical entity to an animal, human or other mammal via any route including but not limited to oral, intradermal, intramuscular, intraperitoneal, or intravenous.

Pharmaceutical formulation. The process by which different chemical substances including but not limited to active drugs are combined and formulated for the treatment of humans.

Sterile formulation. A formulation free of living germs or microorganisms.

Therapeutically effective amount. The mass of active drug in and frequency of administration of a formulation that results in the prevention of the development of symptoms, prevention of development of markers or signs of a disease, prevention of the development of tissue or organ damage, prevention of the progression of a disease, reduction in the severity of a disease, or treatment of disease symptoms as defined above.

Dose range for each individual agent. The range of the mass of active drug in and frequency of administration of a formulation which results in the prevention of the development of symptoms, prevention of the development of a disease, prevention of development of markers or signs of a disease, prevention of the development of tissue or organ damage, prevention of the progression of a disease, reduction in the severity of a disease, or treatment of disease symptoms as defined above.

Regimen. Regimen means dose, frequency of administration, for example twice-per day, daily, weekly, bi-weekly etc., and duration of treatment, for example one day, several days, one week, several weeks, one month, several months, one year, several years, etc.

Loading Dose.

A large initial dose of a substance or series of such doses given to more rapidly achieve a therapeutic concentration in the body. A loading dose can be higher or lower than the maintenance dose. In some instances, therapy is initiated at a loading dose for days, weeks or months in order to rapidly achieve therapeutic levels of the drug or other chemical entity in tissue, then the dose is lowered to the long-term maintenance dose. For hydroxychloroquine sulfate and chloroquine phosphate, the standard dose of 400 mg/day can take 4-6 months to achieve therapeutic tissue levels. Therefore, some physicians use loading doses of hydroxychloroquine sulfate or chloroquine phosphate, for example a dose of at least 600 mg/day (6-8.5 mg/kg/day), at least 800 mg/day, at least 1000 mg/day and up to 1200 mg/day (12-17 mg/kg/d) for 1-16 weeks to more rapidly achieve therapeutic levels in the tissues where it is needed for activity. Desethylhydroxychloroquine (DHCQ) is expected to also accumulate slowly in tissues, such that using, for example, loading doses of at least 600 mg/day (6-8.5 mg/kg/day), at least 800 mg/day, at least 1000 mg/day, and up to 1200 mg/day (12-17 mg/kg/d), up to 1400 mg/day, up to 1600 mg/day, up to 1800 mg/day (18-26 mg/kg/d) for 1-16 weeks may also prove therapeutically beneficial when treating with DHCQ. Loading doses of HCQ for treatment of inflammatory disease are discussed in Furst et al. (Arthritis Rheum. 1999 February; 42(2):357-65. PMID: 10025931).

Unit Dose.

Unit doses (also called dosage forms) are essentially pharmaceutical products in the form in which they are marketed for use, typically involving a mixture of active drug components and nondrug components (excipients), along with other non-reusable material that may not be considered either ingredient or packaging (such as a capsule shell, for example). Depending on the context, multi(ple) unit dose can refer to distinct drug products packaged together, or to a single drug product containing multiple drugs and/or doses. The term dosage form can also sometimes refer only to the chemical formulation of a drug product's constituent drug substance(s) and any blends involved.

Dose Pack.

A premeasured amount of drug to be dispensed to a patient in a set or variable dose and in a package including but not limited to a blister pack or other series of container for the purpose of facilitating a dose regimen. A dose pack can be used to facilitate delivery of an initial and/or loading dose to an individual, followed by a maintenance dose.

Excipient.

An excipient is generally a pharmacologically inactive substance formulated with the active ingredient (“API”) of a medication. Excipients are commonly used to bulk up formulations that contain potent active ingredients (thus often referred to as “bulking agents,” “fillers,” or “diluents”), to allow convenient and accurate dispensation of a drug substance when producing a dosage form. They also can serve various therapeutic-enhancing purposes, such as facilitating drug absorption or solubility, or other pharmacokinetic considerations.

Biomarker (also referred to herein as a “marker”). A biomarker is an objectively measured characteristic that reflects a biological condition, pre-disease state, or disease state including but not limited to molecular, biochemical, imaging, or gross physical measurements.

Imaging biomarker (also referred to herein as an “imaging marker”). A biomarker that is measured or otherwise determined through use of an imaging modality, including but not limited to ultrasound, radiography, computerized tomography, magnetic resonance imaging, or nuclear medical scanning.

Biochemical biomarker (also referred to herein as a “biochemical marker”). A biologic substance that is measured in blood or other tissue as a biomarker. Biological biomarkers of interest include without limitation proteins, nucleic acids, metabolites, fatty acids, peptides, and the like.

Inflammatory biomarker (also referred to herein as an “inflammatory marker”). A biomarker representing an inflamed state. Inflammatory biomarkers of interest include without limitation cytokines, chemokines, high sensitivity C-reactive protein (hs-CRP), erythrocyte sedimentation rate (ESR), expression of mRNA encoding inflammatory mediators, inflammatory cells, imaging biomarkers demonstrating inflammation, and other markers indicative of inflammation.

Reference Range.

A reference range is defined as the set of values within which 95 percent of the normal population falls. It typically refers to the value of a biomarker, and examples of such biomarkers include but are not limited imaging biomarkers, biochemical biomarkers, clinical biomarker, radiographic biomarkers, and other biomarkers.

### Aminoquinolines

Aminoquinolines are derivatives of quinoline, most notable for their use as antimalarial drugs. Representative examples of the aminoquinoline class include, but are not limited to, 4-aminoquinolines, such as amodiaquine, hydroxychloroquine, chloroquine; and 8-aminoquinolines, such as primaquine and pamaquine. The drugs may be formulated as a base, or more usually as a salt. Aminoquinolines include the salts and/or ester thereof.

The phrase “pharmaceutically acceptable salt(s)”, as used herein, means those salts of compounds of the invention that are safe and effective for oral and topical use in mammals and that possess the desired biological activity. Pharmaceutically acceptable salts include salts of acidic or basic groups present in compounds of the invention. Pharmaceutically acceptable salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate (i.e., 1,1′-methylene-bis-(2-hydroxy-3-naphthoate)), aluminum, calcium, lithium, magnesium, potassium, sodium, zinc, and diethanolamine salts and the like, as known in the art.

Aminoquinolines are generally available as oral formulations and have known dosing information. For example, amodiaquine is used at a dose of 200-600 mg (2-6 mg/kg), taken from every 6 hours to daily, with lower doses prescribed for children; HCQ sulfate is used at a maintenance dose of 200-400 mg (2-4 mg/kg/d) once a day; for HCQ sulfate, in certain situations an initial loading dose of 400-600 mg (4-6 mg/kg/d) once a day for weeks or months can be used prior to treatment with the maintenance dose; chloroquine is taken preventively at a dose from about 500 (5 mg/kg) mg to about 1.5 g (15 mg/kg) daily, with appropriately lower doses for individuals with low body weight and for maintenance regimens; primaquine is used at daily doses ranging from 15 mg to 30 mg (0.15-0.3 mg/kg); and pamaquine is taken at a dose of 60 mg (0.6 mg/kg) once daily. One of skill in the art is readily apprised of the pharmacology and accepted dosing for these compounds.

For the present invention, the dose of aminoquinoline will be selected for example at a dose of at least about 50 mg (0.5 mg/kg), at least about 100 mg (1.0 mg/kg), at least about 200 mg (2.0 mg/kg), at least about 250 (2.5 mg/kg) mg and not more than about 2,500 mg (25 mg/kg), not more than about 1000 (10 mg/kg) mg, not more than about 600 mg (6 mg/kg/day).

The use of the aminoquinoline HCQ is preferred, at a dose of at least about 50 mg (0.5 mg/kg), at least about 100 mg (1.0 mg/kg), at least about 200 mg (2.0 mg/kg), at least about 250 (2.5 mg/kg) mg and not more than about 2,500 mg (25 mg/kg), not more than about 1000 (10 mg/kg) mg, not more than about 600 mg (6 mg/kg/day).

In a preferred embodiment, the aminoquinoline is HCQ and is delivered at one of the following once-daily doses: about 100 mg/day (1.43 mg/kg/day), about 150 mg/day (2.14 mg/kg/day), about 200 mg/day (2.86 mg/kg/day), about 250 mg/day (3.57 mg/kg/day), about 300 mg/day (4.28 mg/kg/day), about 325 mg/day (6.5 mg/kg/day), about 350 mg/day (5 mg/kg/day), about 375 mg/day (5.36 mg/kg/day), about 400 mg/day (5.7 mg/kg/day), about 425 mg/day (6.1 mg/kg/day), about 450 mg/day (6.4 mg/kg/day), about 500 mg/day (7.1 mg/kg/day), about 600 mg/day (8.57 mg/kg/day), about 700 mg/day (10 mg/kg/day), about 800 mg/day (11.4 mg/kg/day), or about 1000 mg/day (14.2 mg/kg/day).

The use of desethylhydryxochloroquine (DHCQ) is also preferred, at a dose of about 50 mg/day (0.5 mg/kg/day), about 100 mg/day (1.43 mg/kg/day), about 150 mg/day (2.14 mg/kg/day), about 155 mg/day (2.2 mg/kg/day), about 200 mg/day (2.86 mg/kg/day), about 250 mg/day (3.57 mg/kg/day), about 300 mg/day (4.28 mg/kg/day), about 301 mg/day (4.2 mg/kg/day), about 325 mg/day (6.5 mg/kg/day), about 350 mg/day (5 mg/kg/day), about 375 mg/day (5.36 mg/kg/day), about 400 mg/day (5.7 mg/kg/day), about 425 mg/day (6.1 mg/kg/day), about 450 mg/day (6.4 mg/kg/day), about 465 mg/day (6.6 mg/kg/day), about 500 mg/day (7.1 mg/kg/day), about 600 mg/day (8.57 mg/kg/day), about 700 mg/day (10 mg/kg/day), about 800 mg/day (11.4 mg/kg/day), or about 1000 mg/day (14.2 mg/kg/day).

In another preferred embodiment, the aminoquinoline is DHCQ and is delivered at one of the following once-daily doses: about 100 mg/day (1.43 mg/kg/day), about 150 mg/day (2.14 mg/kg/day), about 155 mg/day (2.2 mg/kg/day), about 200 mg/day (2.86 mg/kg/day), about 250 mg/day (3.57 mg/kg/day), about 300 mg/day (4.28 mg/kg/day), about 310 mg/day (4.4 mg/kg/day), about 325 mg/day (4.6 mg/kg/day), about 350 mg/day (5 mg/kg/day), about 375 mg/day (5.36 mg/kg/day), about 400 mg/day (5.7 mg/kg/day), about 425 mg/day (6.1 mg/kg/day), about 450 mg/day (6.4 mg/kg/day), about 465 mg/day (6.6 mg/kg/day), about 500 mg/day (7.1 mg/kg/day), about 600 mg/day (8.57 mg/kg/day), about 700 mg/day (10 mg/kg/day), about 800 mg/day (11.4 mg/kg/day), about 1000 mg/day (14.2 mg/kg/day), about 1200 mg/day (17.1 mg/kg/day), or about 1500 mg/day (21.4 mg/kg/day).

In some studies HCQ has been demonstrated to potentiate the effects of co-administered agents by altering the pharmacokinetics of the co-administered agent. For example, in the case of co-administration of hydroxychloroquine and methotrexate, the mean area under the concentration-time curve (AUC) for methotrexate was increased and the maximum methotrexate concentration (Cmax) decreased when methotrexate was coadministered with HCQ, compared to methotrexate administered alone. The time to reach Cmaxfor methotrexate administration, tmax, was also increased during the coadministration with hydroxychloroquine. The AUC of HCQ showed no significant difference between any of the dosing occasions (Carmichael, et al. 2002. J. Rheumatol. 29(10):2077-83).

The pharmacokinetic profiles of the currently FDA-approved doses of HCQ and atorvastatin are presented in the below table, which lists the drugs' half-life (T_{1/2}), maximum concentration (Cmax in mg/L), the time it takes to reach Cmax (Tmax in hours), volume distribution (Vd in L), and percent oral bioavailability.

TABLE 1

| | | Cmax | Tmax | | Oral |
|---|---|---|---|---|---|
| | T_{1/2} | (mg/L) | (hours) | Vd (L) | Bioavailability |
| HCQ | 41 days | 144.6 | 2-3 | 605 | 89% |
| Atorvastatin | 7 hours | 16.4 | 1-2 | 381 | 14% |

Retinal Toxicity from HCQ and Aminoquinolines

Assessment of retinal toxicity. Current recommendations for screening for HCQ-mediated and other aminoquinoline-mediated retinal toxicity are described (Marmor et al, Ophthalmology. 2011, 118(2):415-22; Bernstein HN. Sury Ophthalmol. October 1967; 12(5):415-47; Anderson C et al, Retina. 2009; 29(8):1188-92; Michaelides M et al, Arch Ophthalmol. January 2011; 129(1):30-9). The recommendations include performing a baseline examination of patients starting these drugs to serve as a reference point. Annual screening for eye toxicity should begin after 5 years (or sooner, if there are additional risk factors including total cumulative dose of more than 1000 g, maintenance dose >6.5 mg/kg/day, renal insufficiency, liver disease, underlying retinal disease, age older than 60 years of age). Annual screening should include 10-2 automated field tests, along with at least one of the following tests: multifocal electroretinogram (mfERG), spectral domain optical coherence tomography (SD-OCT), or fundus autofluorescence (FAF). Because mf ERG testing is an objective test that evaluates function, it may be used in place of visual field tests. Fundus examinations are advised for documentation, but visible bull's-eye maculopathy is a late change, and the goal of screening is to detect toxicity at an earlier stage. On annual HCQ toxicity screening examination, demonstration of a worsening of the results (deterioration of performance on the test and/or worsening of retinal or macular findings) of the multifocal electroretinogram (mfERG), spectral domain optical coherence tomography (SD-OCT), fundus autofluorescence (FAF), visual field tests, and/or direct visualization of the macula indicates the development of retinal toxicity. Early fundus changes in chloroquine/hydroxychloroquine toxicity include the loss of foveal reflex, macular edema, and pigment mottling that is enhanced with the red-free filter. The appearance of the macula correlates poorly with visual-field testing results. Decreased retinal toxicity (or less retinal toxicity) means that the results of these tests are stable and unchanged from the baseline results obtained in the initial pre-treatment baseline exam.

Perimetry.

Baseline central visual field examination may be useful because the earliest macular changes due to aminoquinoline and HCQ toxicity are nonspecific and may be indistinguishable from age-related changes. The Humphrey 10-2 program (white target) is recommended for confirming defects found by the Amsler grid.

Electroretinography (ERG).

ERG can be full field, focal, or multifocal. Focal ERG techniques can record an ERG response from the foveal and parafoveal regions. mfERG, which is typically available in large clinical centers, is more appropriate for the evaluation of chloroquine and/or hydroxychloroquine toxicity because it generates local ERG responses topographically across the posterior pole and can document a bull's eye distribution of ERG depression. mfERG objectively evaluates function and can be used in place of visual fields.

Spectral Domain Optical Coherence Tomography (SD-OCT). SD-OCT measures peripapillary retinal nerve fiber layer (RNFL) thickness and macular inner and outer retinal thickness in patients with long-term exposure to hydroxychloroquine or chloroquine. OCT is useful to detect peripapillary RNFL thinning in clinically evident retinopathy. In addition, selective thinning of the macular inner retina can be detected in the absence of and before clinically apparent fundus changes.

Histologic findings. In animal studies, the first morphologic changes, which become visible within 1 week after initiation of chloroquine treatment, involve ganglion cells manifesting membranous cytoplasmic bodies. Other neural cells of the retina later show these changes. Reversible changes are present for up to 5 months of therapy. Prolonged therapy resulted in progressive degeneration of the ganglion cells and photoreceptor cell bodies and nuclei with outer segment involvement. The most severe changes tended to be perifoveal, with relative foveal sparing. Abnormalities of the pigment epithelium and choroid were seen only after degeneration of the ganglion cells and photoreceptors was established. All of the observations described were made before any abnormalities became detectable in the fundus or on ERG. Pathologic studies of patients with chloroquine retinopathy are few and are limited to cases with advanced retinopathy. Consistent findings include degeneration of the outer retina, particularly the photoreceptors and the outer nuclear layer, with relative sparing of the photoreceptors in the fovea. Pigment migration into the retina is seen. Pathologic changes in the ganglion cells have been a consistent finding. Sclerosis of the retinal arterioles is variable.

Approach and Considerations for HCQ Retinal Toxicity.

Withdrawal of the medication and shifting to another form of treatment is the standard of care for individuals with HCQ and other aminoquinoline-associated early retinal toxicity or retinal abnormalities. Coordination with the rheumatologist or the dermatologist is warranted for comprehensive care of the patient. If serious toxic symptoms occur from overdosage or sensitivity, it has been suggested that ammonium chloride (8 g daily in divided doses for adults) be administered orally 3-4 times/wk for several months after therapy has been stopped. Acidification of the urine with ammonium chloride increases renal excretion of the 4-aminoquinoline compounds by 20-90%. In patients with impaired renal function and/or metabolic acidosis, caution must be taken.

Recent clinical observations demonstrated that in humans taking conventional doses of HCQ (with 400 mg/day being a common dose), the prevalence of retinal toxicity was 6.8 users per 1,000 (Marmor et al, Ophthalmology. 2011 February; 118(2):415-22). The prevalence was dependent on the duration of HCQ use. Toxicity sharply increased towards 1% after 5-7 years of use. Treatment for >15 years resulted in even higher rates of retinal toxicity.

It is shown herein that the co-administration of atorvastatin with HCQ reduces HCQ-mediated retinal toxicity. The rate of retinal toxicity with long-term treatment with the combination of HCQ and atorvastatin is anticipated to be lower than that reported for treatment with HCQ alone (Marmor et al. describes rates of retinal toxicity for treatment with HCQ alone (Ophthalmology. 2011 February; 118(2):415-22)) when the HCQ is used at a similar effective total cumulative dose, and over a similar time period. HCQ-mediated retinal toxicity is identified based on a worsening of the results (deterioration of performance on the test and/or worsening of retinal or macular findings) on annual screening multifocal electroretinogram (mfERG), spectral domain optical coherence tomography (SD-OCT), fundus autofluorescence (FAF), visual field tests, and/or direct visualization of the macula. Decreased retinal toxicity (or less retinal toxicity) means that for a group of individuals treated with the combination of HCQ and atorvastatin, there will be at least about a 25%, at least about a 35%, at least about a 45%, at least about a 55%, at least about a 65%, at least about a 75%, and may be around or up to about a 50% lower rate of retinal toxicity (e.g. toxicity determined based on worsening of function or performance, or development or worsening of abnormal physical characteristics or findings, of the retina or macula on annual screening test results) as compared to that reported for individuals treated with HCQ alone (or compared to a group of individuals treated with HCQ alone).

Specific measurements to document the rate (incidence) of retinal toxicity in individuals receiving treatment with the combination of HCQ and atorvastatin as compared to individuals taking HCQ alone at a similar effective cumulative dose and over a similar time period include (Marmor, Ophthalmology. 2011 February; 118(2):415-22):

(1) Ophthalmologic Examination. A thorough ophthalmologic dilated fundus examination to examine the retinal macula for evidence of bull's-eye maculopathy. Visible bull's-eye retinopathy indicates that toxicity has persisted long enough to cause RPE degeneration, and is a relatively late finding. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of bull's-eye maculopathy at 5 years, at 10 years, at 15 years, and at 20 years of treatment as compared to treatment with HCQ alone at a similar effective cumulative dose and over a similar time period.
(2) Automated Threshold Visual Fields. Parafoveal loss of visual sensitivity may appear before changes are seen on fundus examination. Automated threshold visual field testing with a white 10-2 pattern (i.e., testing with white targets within 10 degrees of the fovea) gives high resolution within the macular region. The finding of any reproducibly depressed central or parafoveal spots can be indicative of early toxicity. Advanced toxicity will typically show a well-developed paracentral scotoma (with or without central sensitivity loss). The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of depressed central or parafoveal spots at 5 years, at 10 years, at 15 years, and at 20 years of treatment as compared to treatment with HCQ alone at a similar effective cumulative dose and over a similar time period. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of reproducibly depressed central or parafoveal spots at 5 years, at 10 years, at 15 years, and at 20 years of treatment as compared to treatment with HCQ alone at a similar effective cumulative dose and over a similar time period.
(3) Spectral Domain-Optical Coherence Tomography. Optical coherence tomography shows a cross-section of retinal layers in the macula. High-resolution instruments (SD or Fourier domain OCT) can show localized thinning of the retinal layers in the parafoveal region and confirm toxicity. Loss of the inner-/outer-segment line may be an early objective sign of parafoveal damage. Further work is needed to evaluate the sensitivity of SD-OCT relative to visual fields or mfERG, but a number of cases have shown prominent SD-OCT changes before visual field loss; 16, 19-22 SD-OCT testing is rapid and the equipment is available in many offices and clinics. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of localized thinning of the retinal layers in the parafoveal region at 5 years, at 10 years, at 15 years, and at 20 years of treatment as compared to treatment with HCQ alone at a similar effective cumulative dose and over a similar time period.
(4) Fundus Autofluorescence. Autofluorescence imaging may reveal subtle RPE defects with reduced autofluorescence or show areas of early photoreceptor damage (which appear as increased autofluorescence from an accumulation of outer segment debris). It has the advantage over fluorescein angiography of being faster and not requiring dye injection. Some cases have demonstrated FAF abnormalities before visual field loss. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of subtle RPE defects with reduced autofluorescence or areas of early photoreceptor damage at 5 years, at 10 years, at 15 years, and at 20 years of treatment as compared to treatment with HCQ alone at a similar effective cumulative dose and over a similar time period.
(5) Multifocal Electroretinogram. The mfERG generates local ERG responses topographically across the posterior pole and can objectively document localized paracentral ERG depression in early CQ and HCQ retinopathy. mfERG may be more sensitive to early paracentral functional loss than the white 10-2 field. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of localized paracentral ERG depression at 5 years, at 10 years, at 15 years, and at 20 years of treatment as compared to treatment with HCQ alone at a similar effective cumulative dose and over a similar time period.

As compared to the rates of retinal toxicity described in the art, given a similar level of therapeutic activity and time period of dosing for HCQ, treatment with the combination of HCQ+atorvastatin is expected to result in less retinal toxicity as compared to treatment with HCQ used at a similar effective total cumulative HCQ dose, as described above. In one embodiment, substantially without retinal toxicity means that in a patient population analogous to that described by the American College of Opthamolology and Marmor et al (Ophthalmology. 2011 February; 118(2):415-22), in which the retinal toxicity rate approached 1% after 5 years in individuals treated with HCQ alone, the combination of HCQ+atorvastatin is anticipated to reduce the rate of HCQ-mediated retinal toxicity to less that about 0.5% of treated individuals. Retinal toxicity is identified based on worsening of the results (deterioration of performance on the test, and/or development or worsening of physical abnormalities, of retinal or macular physical characteristics or findings) on annual screening multifocal electroretinogram (mfERG), spectral domain optical coherence tomography (SD-OCT), fundus autofluorescence (FAF), visual field tests, and/or direct visualization of the macula examinations. Further, given current assumptions that retinal screening is justified as rates of toxicity approach 1%, the reduction in cumulative rates of retinal toxicity associated with HCQ+atorvastatin therapy can in turn reduce the need for retinal toxicity screening to a single screening at 5 and 10 years, or to entirely negate the need for screening. Finally, due to the ability of atorvastatin to protect individuals against HCQ-mediated retinal toxicity, the combination of HCQ+atorvastatin will enable a higher total cumulative dose of HCQ to be delivered, thereby enabling dosing of HCQ at higher daily doses and/or over a longer period of time which will provide greater efficacy in treating the inflammatory disease.

In another embodiment, substantially without retinal toxicity means that in groups of subjects in which one group is treated with the combination of HCQ+atorvastatin and a second group is treated with HCQ alone (when the HCQ is used at a similar total cumulative dose and over the same time period), that after 5 years of treatment the group treated with the combination of HCQ+atorvastatin will exhibit a 50% lower incidence of retinal toxicity as compared to the group treated with HCQ alone. In another embodiment, substantially without retinal toxicity means that in groups of subjects in which one group is treated with the combination of HCQ+atorvastatin and a second group is treated with HCQ alone (when the HCQ is used at a similar total cumulative dose), that after 10 years of treatment the group treated with the combination of HCQ+atorvastatin will exhibit a 50% lower incidence of retinal toxicity as compared to the group treated with HCQ alone. In another embodiment, substantially without retinal toxicity means that in groups of subjects in which one group is treated with the combination of HCQ+atorvastatin and a second group is treated with HCQ alone (when the HCQ is used at a similar total cumulative dose), that after 15 years of treatment the group treated with the combination of HCQ+atorvastatin will exhibit a 50% lower retinal toxicity as compared to the group treated with HCQ alone. In another embodiment, substantially without retinal toxicity means that in groups of subjects in which one group is treated with the combination of HCQ+atorvastatin and a second group is treated with HCQ alone (when the HCQ is used at a similar total cumulative dose), that after 20 years of treatment the group treated with the combination of HCQ+atorvastatin will exhibit a 50% lower incidence of retinal toxicity as compared to the group treated with HCQ alone.

In addition to reducing the incidence of retinal toxicity as described above, the use of atrovastatin with HCQ can reduce the severity of retinal toxicity when it does occur. Atorvastatin reducing the severity of retinal toxicity means that for an individual taking the combination of atorvastatin and HCQ that develops retinal toxicity that there will be at least about a 25%, at least about a 35%, at least about a 45%, at least about a 55%, at least about a 65%, at least about a 75%, and may be around or up to about a 50% reduction in the severity of the retinal toxicity (e.g. the degree of toxicity determined based on worsening of function or performance, or development or worsening of abnormal physical characteristics or findings, of the retina or macula on annual screening test results) as compared to that reported for individuals treated with HCQ alone.

### Statins

Statins are inhibitors of HMG-CoA reductase enzyme. These agents are described in detail;

for example, mevastatin and related compounds as disclosed in U.S. Pat. No. 3,983,140; lovastatin (mevinolin) and related compounds as disclosed in U.S. Pat. No. 4,231,938; pravastatin and related compounds as disclosed in U.S. Pat. No. 4,346,227; simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional agents and compounds are disclosed in U.S. Pat. Nos. 5,208,258, 5,130,306, 5,116,870, 5,049,696, RE 36,481, and RE 36,520. Statins include the salts and/or ester thereof.

For the purposes of the present invention, an effective dose of a statin in a combination with an aminoquinoline is the dose that, when administered for a suitable period of time, usually at least about one week, and may be about two weeks, or more, up to extended periods of time of months or years, will reduce the progression of the disease. It will be understood by those of skill in the art that an initial dose may be administered for such periods of time, followed by maintenance doses, which, in some cases, will be at a reduced dosage.

The formulation and administration of statins is well known, and will generally follow conventional usage. The dosage required to treat autoimmune disease may be commensurate with the dose used in treating hypercholesterolemia. For example, atorvastatin may be administered in a daily dose of at least about 1 mg, at least about 5 mg, at least about 10 mg, and not more than about 250 mg, not more than about 150 mg, not more than about 80 mg. The use of statins in general and atorvastatin in particular at doses from 1-200 mg (0.01-2.9 mg/kg) are preferred.

In preferred embodiments, the statin is atorvastatin and is delivered at one of the following once-daily doses: about 5 mg/day (0.07 mg/kg/day), about 10 mg/day (0.14 mg/kg/day), about 15 mg/day (0.21 mg/kg/day), about 20 mg/day (0.28 mg/kg/day), about 25 mg/day (0.35 mg/kg/day), about 30 mg/day (0.42 mg/kg/day), about 35 mg/day (0.5 mg/kg/day), about 40 mg/day (0.57 mg/kg/day), about 45 mg/day (0.64 mg/kg/day), about 50 mg/day (0.71 mg/kg/day), about 60 mg/day (0.85 mg/kg/day), about 70 mg/day (1 mg/kg/day), or about 80 mg/day (1.14 mg/kg/day).

The statins can be incorporated into a variety of formulations for therapeutic administration. More particularly, the statins of the present invention can be formulated into pharmaceutical compositions by combining them with appropriate pharmaceutically acceptable carriers or diluents either alone or in combination with an aminoquinoline, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. Oral formulations may be preferred.

Those of skill will readily appreciate that dose levels can vary as a function of the specific compound, the severity of the symptoms, and the susceptibility of the subject to side effects. Some of the specific compounds are more potent than others. Preferred dosages for a given compound are readily determinable by those of skill in the art by a variety of means. A preferred means is to measure the physiological potency of a given compound. The use of combination therapy may allow lower doses of each monotherapy than currently used in standard practice while achieving significant efficacy, including efficacy greater than that achieved by conventional dosing of either monotherapy.

Specific examples of statins useful in the methods of the invention are atorvastatin (LIPITOR™); cerivastatin (LIPOBAY™); fluvastatin (LESCOL™); lovastatin (MEVACOR™); mevastatin (COMPACTIN™); pitavastatin (LIVALO™); pravastatin (PRAVACHOL™); Rosuvastatin (CRESTOR™); simvastatin (ZOCOR™); etc.

### Combinations and Formulations

A combination drug product of the invention, which can be provided as a single formulation or as two separate formulations of the active ingredients, an aminoquinoline and a statin, including without limitation a combination of hydroxychloroquine (HCQ) and atorvastatin. In preferred embodiments the combination provides for a synergistic improvement in disease markers or disease symptoms over the administration of either drug as a single agent.

In some embodiments, the formulation or combination of active agents consists essentially of an aminoquinoline and a statin, including without limitation a combination of HCQ and atorvastatin, i.e. no additional active agents are included in the formulation, although excipients, packaging and the like will be present. In some embodiments the formulation is free of NSAIDs, including aspirin. In some embodiments the formulation is free of folic acid or folate. Importantly, this combination does not require use of an antibiotic, an anti-viral, or an anti-bacterial agent, and in some embodiments the formulation is free of antibiotics, anti-viral, or anti-bacterial agents.

The combination can be defined based on the weight ratio of the two drugs, where the aminoquinoline is usually expressed as the amount of base drug that is present, i.e. not including the weight contribution of the counter ion. Where the aminoquinoline is HCQ and the statin is atorvastatin, the ratio may range from about 2:1 to 60:1, from about 5:1 to 50:1, about 10:1 to 25:1, to 15:1 to 20:1.

The combination can be defined based on the dose ratio of the two drugs, where the aminoquinoline is usually expressed as the amount of base drug that is present, i.e. not including the weight contribution of the counterion. Where the aminoquinoline is HCQ and the statin is atorvastatin, the ratio may range from about 160 mg:80 mg (2.2 mg/kg:1.1 mg/kg) to 600 mg:1 mg (8.6 mg/kg:0.014 mg/kg), from about 500 mg:100 mg (7.1 mg/kg:1.4 mg/kg) to 500 mg:10 mg (7.1 mg/kg:0.14 mg/kg), from about 100 mg:10 mg (1.4 mg/kg:0.14 mg/kg) to 250 mg:10 mg (3.6 mg/kg-0.14 mg/kg), to 150 mg:10 mg (2.1 mg/kg:0.14 mg/kg), to 200 mg:10 mg (2.85 mg/kg:0.14 mg/kg).

In a preferred embodiment, the aminoquinoline is HCQ and the statin is atorvastatin, which are administered in the one of the following once-daily fixed dosages (HCQ base mg:atorvastatin base mg): 800:80, 600:80, 500:80, 465:80, 450:80, 425:80, 400:80, 375:80, 325:80, 310:80, 300:80, 275:80, 250:80, 225:80, 200:80, 155:80 100:80, 800:60, 600:60, 500:60, 465:60, 450:60, 425:60, 400:60, 375:60, 325:60, 310:60, 300:60, 275:60, 250:60, 225:60, 200:60, 155:60 100:60, 800:50, 600:50, 500:50, 465:50, 450:50, 425:50, 400:50, 375:50, 325:50, 310:50, 300:50, 275:50, 250:50, 225:50, 200:50, 155:50, 100:50, 800:45, 600:45, 500:45, 465:45, 450:45, 425:45, 400:45, 375:45, 325:45, 310:45, 300:45, 275:45, 250:45, 225:45, 200:45, 155:45, 100:45, 800:40, 600:40, 500:40, 465:40, 450:40, 425:40, 400:40, 375:40, 325:40, 310:40, 300:40, 275:40, 250:40, 225:40, 200:40, 155:40, 100:40, 800:35, 600:35, 500:35, 465:35, 450:35, 425:35, 400:35, 375:35, 325:35, 310:35, 300:35, 275:35, 250:35, 225:35, 200:35, 155:35, 100:35, 800:30, 600:30, 500:30, 465:30, 450:30, 425:30, 400:30, 375:30, 325:30, 310:30, 300:30, 275:30, 250:30, 225:30, 200:30, 155:30, 100:30, 800:25, 600:25, 500:25, 465:25, 450:25, 425:25, 400:25, 375:25, 325:25, 310:25, 300:25, 275:25, 250:25, 225:25, 200:25, 155:25, 100:25, 800:20, 600:20, 500:20, 465:20, 450:20, 425:20, 400:20, 375:20, 325:20, 310:20, 300:20, 275:20, 250:20, 225:20, 200:20, 155:20, 100:20, 800:15, 600:15, 500:15, 465:15, 450:15, 425:15, 400:15, 375:15, 325:15, 310:15, 300:15, 275:15, 250:15, 225:15, 200:15, 155:15, 100:15, 800:10, 600:10, 500:10, 465:10, 450:10, 425:10, 400:10, 375:10, 325:10, 310:10, 300:10, 275:10, 250:10, 225:10, 200:10, 155:10, 100:10, 800:5, 600:5, 500:5, 465:5, 450:5, 425:5, 400:5, 375:5, 325:5, 310:5, 300:5, 275:5, 250:5, 225:5, 200:5, 155:5, or 100:5.

In another embodiment, the aminoquinoline is desethylhydroxychloroquine (DHCQ) and the statin is atorvastatin, which are administered in the one of the following once-daily fixed dosages (DHCQ base mg:atorvastatin base mg): 800:80, 600:80, 500:80, 465:80, 450:80, 425:80, 400:80, 375:80, 325:80, 310:80, 300:80, 275:80, 250:80, 225:80, 200:80, 155:80 100:80, 800:60, 600:60, 500:60, 465:60, 450:60, 425:60, 400:60, 375:60, 325:60, 310:60, 300:60, 275:60, 250:60, 225:60, 200:60, 155:60 100:60, 800:50, 600:50, 500:50, 465:50, 450:50, 425:50, 400:50, 375:50, 325:50, 310:50, 300:50, 275:50, 250:50, 225:50, 200:50, 155:50, 100:50, 800:45, 600:45, 500:45, 465:45, 450:45, 425:45, 400:45, 375:45, 325:45, 310:45, 300:45, 275:45, 250:45, 225:45, 200:45, 155:45, 100:45, 800:40, 600:40, 500:40, 465:40, 450:40, 425:40, 400:40, 375:40, 325:40, 310:40, 300:40, 275:40, 250:40, 225:40, 200:40, 155:40, 100:40, 800:35, 600:35, 500:35, 465:35, 450:35, 425:35, 400:35, 375:35, 325:35, 310:35, 300:35, 275:35, 250:35, 225:35, 200:35, 155:35, 100:35, 800:30, 600:30, 500:30, 465:30, 450:30, 425:30, 400:30, 375:30, 325:30, 310:30, 300:30, 275:30, 250:30, 225:30, 200:30, 155:30, 100:30, 800:25, 600:25, 500:25, 465:25, 450:25, 425:25, 400:25, 375:25, 325:25, 310:25, 300:25, 275:25, 250:25, 225:25, 200:25, 155:25, 100:25, 800:20, 600:20, 500:20, 465:20, 450:20, 425:20, 400:20, 375:20, 325:20, 310:20, 300:20, 275:20, 250:20, 225:20, 200:20, 155:20, 100:20, 800:15, 600:15, 500:15, 465:15, 450:15, 425:15, 400:15, 375:15, 325:15, 310:15, 300:15, 275:15, 250:15, 225:15, 200:15, 155:15, 100:15, 800:10, 600:10, 500:10, 465:10, 450:10, 425:10, 400:10, 375:10, 325:10, 310:10, 300:10, 275:10, 250:10, 225:10, 200:10, 155:10, 100:10, 800:5, 600:5, 500:5, 465:5, 450:5, 425:5, 400:5, 375:5, 325:5, 310:5, 300:5, 275:5, 250:5, 225:5, 200:5, 155:5, or 100:5.

For demonstrating the synergistic activity of the two drugs and establishing an appropriate fixed-dose ratio for clinical investigation, varying amounts of the two drugs are administered to appropriate animal models of inflammatory disease, either at a time of active disease (following disease onset) or at an early time point representative of pre-clinical disease, and the effect on disease activity or progression is measured. Alternatively, the effects of varying amounts of the two drugs are tested on a cellular response mediating inflammation that may be involved in the pathogenesis of disease.

It is within the level of skill of a clinician to determine the preferred route of administration and the corresponding dosage form and amount, as well as the dosing regimen, i.e., the frequency of dosing. In the preferred embodiment, the combination therapy will be delivered in once-a-day (s.i.d.) dosing. In other embodiments, twice-a-day (b.i.d.) dosing may be used. However, this generalization does not take into account such important variables as the specific type of inflammatory disease, the specific therapeutic agent involved and its pharmacokinetic profile, and the specific individual involved. For an approved product in the marketplace, much of this information is already provided by the results of clinical studies carried out to obtain such approval. In other cases, such information may be obtained in a straightforward manner in accordance with the teachings and guidelines contained in the instant specification taken in light of the knowledge and skill of the artisan. The results that are obtained can also be correlated with data from corresponding evaluations of an approved product in the same assays.

In some embodiments, the aminoquinoline drug is dosed at a higher initial dosing range (dose loading) to ensure more rapid achievement of therapeutic levels in blood and tissue, because this agent is known to have wide distribution and thus an extended terminal half-life. Such loading achieves steady-state blood levels, and increases tissue levels, more rapidly than single-dose daily dosing and results in earlier therapeutic efficacy (Furst et al, Arthritis Rheum. 1999 February; 42(2):357-65). Typical dose loading is in the range of 200-1600 mg/d for weeks to months (2-16 mg/kg/d) of HCQ or an equivalent aminoquinoline. This dose loading is done either alone, administered separately from the statin, or combined with the statin, including use of a “dose pack” with a blister packaging or other mechanism that provides clear information about daily dosing that would facilitate initial dose loading followed by continuation with a stable daily dosing, or other regular dosing intervals sufficient to achieve target drug levels and pharmacodymamic efficacies. The loading dose is typically delivered daily for 1-16 weeks, following which the dose is decreased to the typical maintenance dose of 200-400 mg/d (2-4 mg/kg/d). HCQ, DHCQ and other aminoquinolines can be delivered in once-daily doses (e.g. 400 mg/d orally [4 mg/kg/d]), or in a divided twice-daily dose (e.g. 200 mg/d orally twice per day [for a total of 4 mg/kg/d]).

In one aspect, the present invention provides a unit dosage form of the formulation of the invention. The term “unit dose” or “unit dosage form,” refers to physically discrete units suitable as unitary dosages for human subjects, each unit containing a predetermined quantity of drugs in an amount calculated sufficient to produce the desired effect in association with a pharmaceutically acceptable diluent, carrier, or vehicle. The specifications for the unit dosage forms of the present invention depend on the particular combination employed and the effect to be achieved, and the pharmacodynamics associated with the host.

In one aspect, the agents are formulated together to assure that a therapeutic level of HCQ is achieved concurrently with atorvastatin by effecting an over-encapsulation of the statin with the aminoquinoline, thus allowing staged dissolution of the agents.

In one aspect, the agents are formulated together such that HCQ co-administration results in a lower maximum concentration (Cmax) of atorvastatin but a greater area under the concentration-time curve (AUC), thus increasing efficacy and decreasing toxicity of atorvastatin and allowing dosing lower than current conventional dosing, as has been demonstrated for other co-administered molecules (Carmichael, et al. 2002. J. Rheumatol. 29(10):2077-83).

In one aspect, the agents are formulated together into pharmaceutical compositions by combination with appropriate, pharmaceutically acceptable carriers or diluents, and are formulated into preparations in solid, semi-solid, liquid, suspension, emulsion, or gaseous forms, such as tablets, capsules, powders, granules, ointments, solutions, suspensions, emulsions, suppositories, injections, inhalants, gels, microspheres, and aerosols. As such, administration can be achieved in various ways, usually by oral administration. In pharmaceutical dosage forms, the drugs may be administered in the form of their pharmaceutically acceptable salts, or they may also be used alone or in appropriate association, as well as in combination with other pharmaceutically active compounds. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch, or potato starch; with binders, such as crystalline cellulose, cellulose derivatives, acacia, corn starch or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Pharmaceutically acceptable excipients, such as vehicles, adjuvants, carriers or diluents, are commercially available. Moreover, pharmaceutically acceptable auxiliary substances, such as pH-adjusting and buffering agents, tonicity-adjusting agents, stabilizers, wetting agents and the like, are commercially available. Any compound useful in the methods and compositions of the invention can be provided as a pharmaceutically acceptable base-addition salt. “Pharmaceutically acceptable base-addition salt” refers to those salts that retain the biological effectiveness and properties of the free acids, which are not biologically or otherwise undesirable. These salts are prepared by adding an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline, and caffeine.

The active agent, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The active agents of the present invention or salts thereof may form a solvate and/or a crystal polymorph, and the present invention contains such solvates and crystal polymorphs of various types. A solvate means a solvate of the compound of the present invention or its salt, and example includes solvate of which solvent is alcohol (e.g., ethanol), hydrate, or the like. Example of hydrate includes mono-hydrate, dihydrate or the like. A solvate may be coordinated with an arbitrary number of solvent molecules (e.g., water molecules). The compounds or salts thereof may be left in the atmosphere to absorb moisture, and a case where adsorbed water is attached or a case where hydrate is formed may arise. Moreover, the compounds or salts thereof may be recrystallized to form their crystal polymorph.

As used herein, compounds that are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Although specific drugs are exemplified herein, any of a number of alternative drugs and methods apparent to those of skill in the art upon contemplation of this disclosure are equally applicable and suitable for use in practicing the invention. The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry, and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass humans having pre- or early-stage inflammatory disease. Subjects may be human, but also include other mammals, particularly those mammals useful as laboratory models for human disease, e.g. mouse, rat, cats, dogs, horses, etc.

The expression “body fluid” as used herein in intended to include all of those accessible body fluids usable as clinical specimens which may contain a compound being tested for in sufficient concentration in said fluid to be within the limits of detection of the test device or assay being used. Body fluids will thus include whole blood, serum, plasma, urine, cerebrospinal fluid, synovial fluid, and interstitial and other extracellular fluids, particularly synovial fluid of affected joints. In some embodiments a body fluid used for determination of a marker of early-stage inflammation is a synovial fluid from a joint suspected of being involved in early arthritis. In other embodiments a body fluid used for marker determination is systemic, e.g. blood, urine, etc.

Care should be exercised in the collection and storage of the fluids to be tested. Steps should be taken to avoid proteolysis of the compounds to be tested for in said fluids, and freezing of the fluids is usually warranted unless the test involved can be carried out within a shortly after the fluids are collected. It is usually preferable to use synovial fluid rather than serum because of the likelihood that there will be greater concentrations of the compounds being tested for in the synovial fluid. On the other hand, increased levels of viscosity in synovial fluids pose problems in immunoassay systems that must be addressed by the artisan. It may be preferable to conduct longitudinal studies of a selection of cytokines and markers as well as their respective inhibitors and binding proteins in order to obtain the most accurate profile possible in determining whether an individual is in the early stages of articular cartilage degeneration and is therefore a candidate for intervention with the methods of the invention.

### Prevention and Treatment

The methods of the invention can be used for prophylactic as well as therapeutic purposes.

As used herein, in one embodiment the term “treating” refers to prophylactic or preventative use of the intervention in individuals with increased risk for or with early-stage inflammatory disease. In such individuals, treatment prevents development of symptoms or signs of disease, prevents development of disease, and/or reverses signs or symptoms of disease. In another embodiment, the term “treating” refers to treating individuals with established disease to reduce symptoms or signs of disease, to prevent disease progression, and/or to reverse symptoms of signs of disease.

Individuals at increased risk for or with early stages of an inflammatory disease are generally asymptomatic, and exhibit no or minimal symptoms and signs of the disease. In some embodiments, individuals at increased risk for developing an inflammatory disease are treated with the combination of hydroxychloroquine and atorvastatin to prophylactically prevent them from developing signs of an inflammatory disease, symptoms of an inflammatory disease, or the inflammatory disease. In some embodiments, individuals at increased risk for developing an inflammatory disease are treated with the combination of hydroxychloroquine and atorvastatin to prevent them from exhibiting progression of signs of an inflammatory disease or symptoms of an inflammatory disease, and/or to prevent them from developing the inflammatory disease. Thus, the invention provides a significant advance in the treatment of at-risk individuals, individuals with pre-clinical findings, or individuals with early-stage disease, by preventing the development of clinical symptoms or signs of a disease or by preventing the progression of the clinical symptoms or signs of a disease. Such treatment is desirably performed prior to the development of clinical symptoms or signs of disease, and before significant loss of function in the affected tissues, i.e. in the “at increased risk” for or “early-stage” inflammatory disease states.

This invention specifically provides for the treatment of humans and other mammals that have pre-clinical or early-stage inflammatory disease but are asymptomatic, or have early and mild symptoms or signs of the disease. In such asymptomatic individuals with pre-clinical or early-stage inflammatory disease, this invention can prevent the development of symptomatic inflammatory disease, prevent the development of signs of the disease, or reduce the progression of early-symptomatic inflammatory disease. In individuals with early symptoms of signs of inflammatory disease, with such early symptoms and signs being present for less than 6 months or being mild in severity, this invention can prevent the development of the full symptoms of an inflammatory disease, prevent the development of signs and features of the disease, or reduce the progression of early-stage inflammatory disease.

An example of a common treatment used in preventing the development of disease in an individual with pre-clinical or early-stage disease is statin therapy for hypercholesterolemia. Statin therapy is used to treat asymptomatic individuals with hypercholesterolemia, and thus exhibiting pre-clinical disease based on the presence of hypercholesterolemia and normal coronary arteries, or early-stage disease based on the presence of hypercholesterolemia and early-stage atherosclerosis of the coronary arteries. Humans with pre-clinical or early-stage atherosclerotic disease have asymptomatic hypercholesterolemia and are at increased risk for developing symptomatic atherosclerosis and coronary artery disease that can manifest as angina and/or a myocardial infarction. Because of the effectiveness and excellent safety profile of statin therapy, and the severe nature of symptomatic atherosclerosis and coronary disease that can result in myocardial infarction, treatment of asymptomatic individuals with hypercholesterolemia is now “standard of care” in medical practice. In an analogous fashion, an aspect of this invention is the treatment of asymptomatic individuals with pre-clinical or early-stage inflammatory disease to prevent them from developing symptomatic inflammatory disease.

This invention specifically provides for the treatment of humans and other mammals that have early-stage (which in certain cases and diseases can have mild symptoms, or intermittent symptoms, or symptoms for less than 6 months) or established-inflammatory disease. In such symptomatic individuals with early-stage or established inflammatory disease, this invention can prevent progression of or reduce the severity of the symptoms and signs of the inflammatory disease.

In one embodiment, treatment of individuals at increased risk for development of an inflammatory disease reduced their overall risk for development of the inflammatory disease. Decreasing an individual's risk for development of an inflammatory disease means that for an individual or a group of individuals treated with the combination of HCQ and atorvastatin, there will be at least about a 25%, at least about a 35%, at least about a 45%, at least about a 55%, at least about a 65%, at least about a 75%, and may be around or up to about a 50% lower rate of development of the inflammatory disease as compared to the rate of development of the inflammatory disease in individuals not treated with HCQ and atorvastatin (either previously described in the literature for a patient population with similar characteristics, or for individuals treated with alternative therapies).

Developing an inflammatory disease means being formally diagnosed with the inflammatory disease by a physician. Further, developing an inflammatory disease means developing the symptoms, physical exam findings, laboratory test findings, imaging findings, biomarker findings, and other findings that meet the established diagnostic criteria for the inflammatory disease and thereby enable a physician to diagnose an individual with the inflammatory disease.

The expression “presently or prospectively” as used herein is intended to mean that in accordance with the methods discussed below of making that determination, it is possible to identify an individual as either being presently in need of such treatment, or very likely or expected to be in need of such treatment in the near-term future. Prospective need of treatment may be established by those determinations of positive factors that from the experience of the artisan lead directly to the early stages of an inflammatory disease.

The expression “the early stages of inflammatory disease” is intended to mean the very beginning of the initial pathologic changes. Said pathologic changes include changes in the composition, form, density, signs and/or inflammatory state of the involved tissue or organ from that present in healthy individuals.

Individuals at increased risk for or with early-stage inflammatory disease can be treated with a combination therapy of the invention to prevent the development of disease, to prevent development of signs of the disease, to prevent the onset of symptomatic disease, to prevent progression of signs or symptoms of disease, or to prevent progression of inflammation. The aminoquinoline and statin can be delivered in individual tablets or capsules, or in a combined tablet or capsule that includes both drugs. Importantly, this novel use of this combination does not require use of an antibiotic, an anti-viral or an anti-bacterial agent. No antibiotic, anti-viral, or anti-bacterial compound is needed for the anti-inflammatory activity and disease-modifying activity described herein.

In another embodiment, this invention is for the treatment of individuals with established inflammatory disease. The inflammatory disease is diagnosed based on an individual exhibiting symptoms, signs, clinical features, laboratory test results, imaging test results, biomarker results, and other findings that enable a physician to formally diagnose that individual with the inflammatory disease. In some embodiment, established inflammatory disease is an inflammatory disease for which an individual has had a formal diagnosis of the disease made by a physician for longer than 6 months. In established inflammatory disease, the signs or symptoms of disease may be more severe. In established inflammatory disease, the disease process may cause tissue or organ damage.

Individuals at increased risk for development of an inflammatory disease, with early-stage inflammatory disease, or with established inflammatory disease can be treated with a combination of the invention to prevent the development of disease, to prevent the progression of disease, and to prevent the progression of the symptoms or signs of disease. The dose of aminoquinoline is generally 400 mg/day, but can be between 25-3,000 mg/day. The dose of atorvastatin is generally 10, 20, 30 or 40 mg/day, but can be between 5 and 80 mg/day. The aminoquinoline can be delivered alone, or in combination with atorvastatin can be delivered in individual tablets or capsules or in a combined tablet or capsule that includes both drugs. Inflammatory diseases include autoimmune diseases including multiple sclerosis, rheumatoid arthritis, Crohn's disease, psoriasis and other autoimmune diseases; degenerative diseases including osteoarthritis, Alzheimer's disease, macular degeneration and other degenerative diseases; metabolic diseases including type II diabetes, coronary artery disease, metabolic syndrome and other metabolic diseases; chronic infections that result in inflammation including human immunodeficiency virus infection, hepatitis C virus infection, cytomegalovirus infection, and other viral, bacterial, fungal, parasite and other infection; and other inflammatory diseases such as fatty liver disease.

Treatment and Determination of an Individual with Pre-Clinical or Early-Stage OA

The present invention provides a method of treating or preventing degeneration or destruction of articular cartilage or remodeling of the subchondral bone in the joints of an individual in need of such treatment, comprising establishing the status of an individual as presently or prospectively being in said early stages and thus in need of such treatment; and administering to the individual a combined therapy of the invention in an amount therapeutically effective for treating or preventing said degeneration or destruction of articular cartilage or subchondral bone. In some embodiments the criteria for treatment further includes evidence of inflammation in the affected joint.

Assessment of OA may use the Kellgren Lawrence (KL) grading system (Kellgren and Lawrence, Ann. Rheum. Dis., 16:494-502, 1957, herein specifically incorporated by reference). The KL grading system relies on an anterior-posterior (AP) radiograph and is as follows: grade 0=no features of OA; grade 1=presence of OA is doubtful, presence of minute osteophyte(s), unchanged joint space; grade 2=minimal OA, definite osteophyte(s), unchanged joint space; grade 3=moderate OA, moderate diminution of joint space; grade 4=severe OA, joint space greatly reduced with sclerosis of subchondral bone. For the purposes of the present invention, the KL score is less than 3, in some embodiments less than 2, and desirably less than one.

Use of the combination therapies described herein is aimed at intervention during the pre-clinical or early stages of OA, during which there is evidence of only mild cartilage abnormalities or lesions as defined by the presence of at least one imaging marker indicative of pre-clinical or early-stage OA, as determined by imaging or direct visualization modalities, molecular marker analysis, or clinical history of a condition or event predisposing to the development of OA. The combination therapy of the invention modifies OA disease progression as measured by either stabilization of KL score and/or joint-space narrowing, or prevention of further cartilage breakdown (as assessed by imaging using MRI or another imaging modality), or reduction in levels of molecular markers of cartilage breakdown.

Individuals with pre-clinical or “pre-OA” are those at increased risk of developing OA, as evidenced by biochemical, imaging, or clinical markers. Conditions or events that predispose to the development of OA include, without limitation, a history of injury to a joint; clinically or radiographically diagnosed meniscal injury with or without surgical intervention; a ligamentous sprain with clinically or radiographically diagnosed anterior or posterior cruciate or medial or lateral collateral ligament injury (Chu et al, Arthritis Res Ther. 2012 14(3):212. PMID: 22682469); clinically measured limb-length discrepancy; obesity with a current, or prolonged historical period of, BMI >27; or biomechanical features of abnormal gait or joint movement. In general, a determination of pre-clinical OA is associated with one or more, two or more, three or more parameters of joint pathology including, without limitation and relative to a healthy control sample, cartilage proteoglycan loss; cartilage damage; or elevated levels of degradative enzymes, the presence of products of cartilage or extracellular matrix degradation or bone remodeling. Humans at risk for OA, who have pre-OA, and who have early-stage OA are often asymptomatic, but a subset of patients experience joint pain due to cartilage injury (e.g. meniscal injury), ligamentous injury (e.g. tearing of the anterior cruciate ligament), or another joint abnormality. The joint pain in individuals with pre-OA and early-stage OA is generally intermittent and mild in nature.

Markers indicative of pre-clinical OA. Compared to the joints of healthy control individuals, a joint in an individual with pre-clinical OA will exhibit a KL score of 0, and have one, two, three, four or more markers indicative of pre-clinical disease. MRI-detected imaging markers indicative of the presence of pre-clinical OA include cartilage edema, cartilage proteoglycan loss, cartilage matrix loss, bone marrow edema, articular cartilage fissures, articular cartilage degeneration, a meniscal tear, an anterior cruciate ligament tear, a posterior cruciate ligament tear, and other abnormalities of the cartilage or ligaments in the joint. Ultrasound will show evidence of cartilage edema or damage. Arthroscopy can allow direct detection or visualization of cartilage edema, cartilage softening, cartilage thinning, cartilage fissures, cartilage erosion, or other cartilage abnormalities. Cartilage damage is frequently defined by the Outerbridge classification criteria or similar directly observed changes within the joint. For example, one such scoring system defines the presence of damage is as follows: grade 0=normal cartilage; grade I: softening and swelling of cartilage; grade II: a partial-thickness defect in the cartilage with fissures on the surface that do not reach subchondral bone or exceed 1.5 cm in diameter; grade III: fissures in the cartilage that extend to the level of subchondral bone in an area with a diameter of more than 1.5 cm. Humans at risk for OA or with “pre-clinical OA” may be asymptomatic or have mild symptoms, with have a KL score of 0, but may have signs of cartilage damage, meniscal damage, ligament damage, or other abnormalities of the joint based on MRI imaging, ultrasound imaging, or direct visualization of the joint on arthroscopy.

Markers indicative of early-stage OA. As compared to joints in healthy individuals, a joint in an individual with early-stage OA will typically exhibit a KL score of 0 or 1, and have one, two, three, four or more markers indicative of early disease. Plain X-rays of the involved joint would demonstrate features consistent with a KL score of 0-2, including no osteophytes or small osteophytes, and no or minimal joint space narrowing. MRI-detected imaging markers indicative of early-stage OA include cartilage proteoglycan loss, cartilage thinning, cartilage fissures or cartilage breakdown. Ultrasound will show evidence of cartilage edema or damage. Arthroscopy can provide for direct detection or visualization of cartilage edema, cartilage softening, cartilage thinning, cartilage fissures, cartilage erosion, or other cartilage abnormalities. Cartilage damage is frequently defined by the Outerbridge classification criteria or similar direct observational changes within the joint. Humans with early OA may be asymptomatic, or may have mild or intermittent symptoms, or may have symptoms for less than 6 months, but may exhibit findings associated with cartilage damage as represented by Outerbridge grade 0, grade I and grade II scores or similar direct observational changes within the joint, as well as with other cartilage, meniscal and ligament damage based on MRI imaging, ultrasound imaging, or direct visualization of the joint on arthroscopy.

Established and Advanced OA.

In contrast to pre-clinical OA and early-stage OA, advanced OA can be defined radiographically as KL grade >=2 or as MRI evidence of extensive, complete, or near-complete loss of articular cartilage. Other evidence of joint failure can be determined by direct or arthroscopic visualization of extensive, complete, or near-complete loss of joint space or cartilage, by biomechanical assessment of inability to maintain functional joint integrity, or by clinical assessment of joint failure, as evidenced by inability to perform full range of motion or to maintain normal joint function. On physical examination, patients with advanced OA can have bony enlargement, small effusions, crepitus, and malalignment of the synovial joints. Examples of semiquantitative MRI scoring systems that can be used to classify the severity of OA include: WORMS (Whole-Organ Magnetic Resonance Imaging Score; Peterfy C G, et al. Osteoarthritis Cartilage 2004; 12:177-190); KOSS (Knee Osteoarthritis Scoring System; Kornaat P R, et al. Skeletal Radiol 2005; 34:95-102); BLOKS (Boston Leeds Osteoarthritis Knee Score; Hunter D J, et al. Ann Rheum Dis 2008; 67:206-211); MOAKS (MRI Osteoarthritis Knee Score; Hunter D J, et al. Osteoarthritis Cartilage. 2011; 19(8):990-1002); HOAMS (Hip Osteoarthritis MRI Score; Roemer F W, et al. Osteoarthritis Cartilage. 2011; 19(8):946-62); OHOA (Oslo Hand Osteoarthritis MRI Score). Advanced OA can result in significant joint pain and loss of mobility owing to joint dysfunction.

In a preferred embodiment, the individual treated by the methods of the invention has pre-clinical or early-stage OA or RA. In other embodiments, the individual treated by the methods of the invention has established OA, RA or other type of arthritis.

### Assessing Inflammation in Pre-Clinical OA, Early-Stage OA, and Advanced OA.

A variety of markers can be used to assess inflammation in pre-clinical OA, early-stage OA, and advanced OA, including imaging markers, molecular markers, and clinical markers. Examples of clinical markers include the presence of a joint effusion on physical examination. Another example of a clinical marker is the presence of morning stiffness in the joint. Examples of imaging markers include the use of MRI or ultrasound-detected signs of inflammation in the joint. MRI can be performed either with or without gadolinium contrast, and MRI-evidenced inflammation is defined as the presence of one or more of the following findings: synovitis (synovial lining thickening, proliferation, and/or enhancement (increased signal), including a positive Doppler-flow signal in the synovial lining), joint effusion, bone marrow edema, etc (Krasnokutsky et al, Arthritis Rheum. 2011 63(10):2983-91. doi: 10.1002/art.30471 PMID: 21647860; Roemer et al, Osteoarthritis Cartilage. 2010 October; 18(10):1269-74. PMID: 20691796; Guermazi et al, Ann Rheum Dis. 2011 70(5):805-11, PMID: 21187293). Ultrasound-evidenced inflammation is defined as the presence of one or more of the following findings: synovial lining thickening and/or enhancement, a joint effusion, bone marrow enhancement, etc. (Guermazi et al, Curr Opin Rheumatol. 2011 23(5):484-91. PMID: 21760511; Hayashi et al, Osteoarthritis Cartilage. 2012 March; 20(3):207-14. PMID: 22266236; Haugen et al, Arthritis Res Ther. 2011; 13(6):248. PMID: 22189142). Molecular markers that can be used to assess inflammation include erythrocyte sedimentation rate (ESR), CRP, cytokines, chemokines, and other inflammatory mediators. ESR and CRP are measured in blood, and the other molecular markers of inflammation can be measured in blood or synovial fluid.

In one embodiment, one or more of these inflammatory markers including physical exam markers, imaging (MRI findings, ultrasound findings) markers, laboratory biomarkers (CRP, ESR), and other biomarkers are used to identify individuals with active inflammation that are most likely to respond to treatment with the combination of an aminoquinoline and a statin. In another embodiment, individuals with degenerative meniscal tear of the knee are subjected to MRI analysis of the knee and hs-CRP laboratory testing. If the MRI synovitis score (Guermazi et al., Ann Rheum Dis. 2011 70(5):805-11. PMID: 21187293) is >5 or the hs-CRP is >2.5 mg/L, then the individual is treated with the combination of HCQ and atorvastatin. In another embodiment, individuals at increased risk for knee OA who experience intermittent knee pain are subjected to MRI analysis of the knee and hs-CRP laboratory testing. If the MRI synovitis score (Guermazi et al., Ann Rheum Dis. 2011 70(5):805-11. PMID: 21187293) is >5 or the hs-CRP is >2.5 mg/L, then the individual is treated with the combination of HCQ and atorvastatin.

In another embodiment, one or more of these same inflammatory markers is used to monitor an individual's response to treatment, to determine if treatment should be continued, or to determine if treatment can be discontinued. For example, individuals at increased risk for OA who are being treated with the combination of HCQ and atorvastatin are monitored annually, or every-other year, by MRI and hs-CRP. Individuals, whose MRI synovitis score declines to below 3 or whose hs-CRP declines to below 1 mg/L are identified as having exhibited a positive response to therapy and that their at-risk state, early-disease state, or established disease state has responded well to treatment.

Determination of an Individual with “Pre-Clinical” (Pre-RA) or Early-Stage RA

Individuals at increased risk for the development of RA, or with “pre-clinical RA”, or with early-stage RA, are identified based on the presence of biochemical, imaging, or clinical markers indicative of RA. Findings that suggest an individual has early-stage RA include one or more of the following: presence of one or more swollen joints, presence of anti-CCP or RF antibodies, evidence of synovial enhancement (increased signal) on MRI scan or ultrasound, elevated levels of autoantibodies or cytokines that have can predict the development of RA (as described in Sokolove et al, PLoS One. 2012; 7(5):e35296; Deane et al, Arthritis Rheum. 2010 62(11):3161-72; Gerlag et al, Ann Rheum Dis. 2012 71(5):638-41). Factors that increase an individual's risk of developing RA include one or more of the following: a family history of RA (particularly in a first-degree relative), increased levels of anti-CCP and/or RF autoantibodies, a genetic profile associated with susceptibility to RA, and cigarette smoking (as described in Deane et al, Rheum Dis Clin North Am. 2010 36(2):213-41; Klareskog et al, Semin Immunol. 2011 April; 23(2):92-8).

At Increased Risk for Developing RA and Pre-Clinical-RA.

Individuals are classed as being at risk of developing RA on the basis of their having specific biochemical, serologic, genetic, imaging, or clinical markers. The pre-clinical phase of RA is characterized by the presence of immunologic markers of RA, including the development of anti-citrullinated protein antibodies (ACPA) and rheumatoid factor (RF) years before the onset of clinically apparent RA. As the onset of clinical apparent disease approaches, the ACPA response spreads, i.e., there is an increase in number of levels of autoantibodies targeting citrullinated proteins. Additionally, there is often a concomitant rise in the level of serum cytokines and chemokines as well as acute phase reactants (including but not limited to ESR and CRP) (Sokolove et al, PLoS One. 012; 7(5):e35296. 2012, PMID: 22662108; Deane et al, Arthritis Rheum. 2010 November; 62(11):3161-72). Thus, “at risk” and “pre-clinical” RA can be defined by the presence of the molecular markers ACPA, RF, elevated cytokines, or combinations of these markers. Additionally, pre-clinical RA including “at risk” could be defined by genetic markers and/or family history. Such genetic markers include but are not limited to the HLA DR4 shared epitope and other genetic polymorphisms, such as PTPN22, PAD4, STAT4, and TRAF1-05.

Early-Stage RA.

Early-stage RA is rarely asymptomatic; it most often manifests as pain in and/or stiffness of the small or medium joints, and it can be associated with joint swelling or synovitis. Early-stage RA can be defined by the presence of signs and symptoms consistent with RA of less than 3-6 months duration and lack of radiographic joint damage as determined by plain X-ray. Early-stage RA is also indicated by the presence of imaging markers (determined, for example, by MRI or ultrasound, including increased Doppler-flow signal on ultrasound), such as synovial enhancement, bone marrow edema, an effusion, or other findings indicative of inflammation (Gerlag et al, Ann Rheum Dis. 2012 71(5):638-41. PMID: 22387728).

Advanced RA.

Advanced RA is can be defined as RA of greater than 3-6 months duration and often at last 1 year duration. Radiographic signs of RA, such as periarticular erosions of the bone, can be detected within 1-2 years of disease onset, and therefore an alternative definition of advanced RA may include evidence of radiographic joint-space narrowing and/or erosions.

Determination of an Individual with “Pre-Clinical” or Early-Stage Multiple Sclerosis (MS)

Multiple Sclerosis.

Multiple sclerosis is an autoimmune neurologic condition caused by demyelination of neurons as a result of immune injury. It is caused by a direct immunologic attack, mediated by autoreactive T cells and B cells, on protein and lipid components of the myelin sheath.

Pre-Clinical MS.

Individuals with pre-clinical MS are those at increased risk of developing MS, as indicated by biochemical, serologic, genetic, imaging, or clinical parameters. The pre-clinical phase of MS can be characterized by the presence of immunologic markers associated with the later onset of MS, for example autoantibodies that appear several years before the onset of clinically apparent MS. Additionally or alternatively, individuals with pre-clinical MS can have neurologic signs and/or symptoms that alone do not diagnose MS but may be associated with the later onset of clinically apparent MS. Such signs or symptoms include but are not limited to optic neuritis (which generally manifests as loss of vision or decreased vision in one eye), numbness, dizziness, muscle spasms. Symptoms of pre-clinical MS are typically of limited duration but can wax and wane. They may be associated with radiographic changes including but not limited to white-matter lesions as determined by MRI, which often appear as bright areas on T2-weighted MRI. Additionally, pre-clinical MS can be associated with the presence of cerebrospinal fluid (CSF) abnormalities including abnormally high numbers of white blood cells or levels of protein, and/or the presence of oligoclonal bands.

Thus, pre-clinical MS can be defined as the presence of clinical symptoms of early demyelination and/or by the presence of specific autoantibodies in serum or CSF, abnormally high levels of protein or white blood cells in CSF, brain or spinal cord lesions detected by imaging, or combinations of these markers.

Early-Stage MS.

Early-stage MS most often manifests as persistent or recurrent neurologic symptoms of demyelination, including but not limited to focal or multifocal numbness, tingling, weakness, loss of balance, or compromised vision including blurry or double vision. Definitive diagnosis of MS requires evidence of 2 or more brain lesions detected by MRI and/or 2 or more episodes of neurologic symptoms lasting at least 24 hours and occurring at least one month apart.

Advanced MS.

Advanced MS can be defined as MS that has progressed to permanent neurologic disability, usually with non-resolving lesions as detected by MRI. Additionally, MS symptoms may wax and wane in a pattern known as relapsing-remitting MS. This pattern can be seen late into the course of MS, with or without continued accrual of damage in a chronic progressive pattern in which disease progresses with increasing neurologic symptoms without complete recovery from prior lesions.

Determination of an Individual with “Pre-Clinical” or Early-Stage Cardiovascular Disease

Atherosclerotic Cardiovascular Disease.

Atherosclerosis is characterized by accumulation of fatty materials in the arterial wall, resulting in development of fatty plaques, which may rupture and cause vascular occlusion and ischemia. The lesion of atherosclerosis comprises a highly inflammatory milieu characterized by the accumulation of inflammatory cells, including macrophages and to a lesser extent T and B cells, and production of high levels of inflammatory cytokines, chemokines, and MMPs (Libby et al, Nature 2011. 473(7347):3170-25. PMID#21593864). Atherosclerosis is associated with and likely promoted by low-grade inflammation.

Individuals at risk for the development of atherosclerosis are those with known risk factors for atherosclerotic coronary artery disease. Risk factors include traditional risk factors for atherosclerotic heart disease, such as those described in the Framingham Risk Score, including high blood pressure, cigarette smoking, elevated levels of HDL cholesterol, glucose intolerance, increased age, male sex, and other factors (see D'Agostino R B Sr, Vasan R S, Pencina M J, Wolf P A, Cobain M, Massaro J M, Kannel W B. Circulation. 2008 Feb. 12; 117(6):743-53. PMID: 18212285).

Early-Stage Atherosclerosis.

Early-stage atherosclerosis is characterized by early changes in coronary arteries, cerebral arteries, and/or other arteries. Such arterial abnormalities can be visualized through imaging using MRI, CT, angiography, or other methods. Because such early-stage disease does not occlude the involved blood vessels, individuals are asymptomatic and they exhibit normal exercise (treadmill or bicycle) or chemical (persanthine or adenosine or dobutamine) stress test results (based on readouts using radiographic contrast and/or electrocardiogram (EKG) changes suggestive of ischemia).

Advanced Atherosclerosis.

Advanced atherosclerosis is characterized by symptomatic heart or cardiovascular disease, including angina, myocardial infarction, transient ischemic attacks, and/or stroke due to arterial occlusion. Advanced atherosclerosis manifests as more advanced arterial abnormalities that can be visualized through imaging using MRI, CT, angiography, and other methods. In addition, with advanced atherosclerosis functional testing with an exercise (treadmill or bicycle) or chemical (persanthine or adenosine or dobutamine) stress test results findings suggestive of ischemia detected by radiographic contrast and/or electrocardiogram (EKG).

In one embodiment, one or more inflammatory markers and other biomarkers are used to identify individuals at increased risk for atherosclerotic disease with active inflammation who are likely to respond to treatment with the combination of an aminoquinoline and a statin. In another embodiment, individuals at increased risk for atherosclerotic disease with increased blood cholesterol (total cholesterol >250 mg/dL or LDL >150 mg/dL) are subjected to hs-CRP laboratory testing. If the hs-CRP is >3, then the individual is determined to be at high-risk for progression of atherosclerotic heart disease and is treated with the combination of HCQ and atorvastatin.

In another embodiment, hs-CRP is used to monitor an individual's response to treatment with the combination of HCQ and atorvastatin, to determine if the individual who is at increased risk for atherosclerotic disease has responded to treatment and/or if the treatment should be continued. For example, individuals at increased risk for atherosclerotic who are being treated with the combination of HCQ and atorvastatin are monitored annually, or every-other year, by repeat cholesterol and hs-CRP testing. Individuals, whose total cholesterol declines below 220, LDL cholesterol declines to below 120, and whose hs-CRP declines to be below 1 are identified as having exhibited a positive response to therapy and that their at-risk state, early-disease state, or established disease state is well-controlled by combination therapy with HCQ and atorvastatin.

Determination of an Individual with “Pre-Clinical” or Early-Stage Type II Diabetes

Type II Diabetes Mellitus and Metabolic Syndrome.

Type II diabetes mellitus is characterized by the presence of insulin resistance and hyperglycemia, which may lead to retinopathy, nephropathy, and neuropathy. Metabolic syndrome refers to a group of factors, including hypertension, obesity, hyperlipidemia, and insulin resistance (manifesting as frank diabetes or high fasting blood glucose or impaired glucose tolerance), that raises the risk of developing heart disease, diabetes, or other health problems. There is a progression from a phase of normal metabolic status to one of impaired fasting glucose (IFG: fasting glucose blood glucose levels greater than 100 mg/dL) or impaired glucose tolerance (IGT: two-hour glucose levels of 140 to 199 mg/dL after a 75-gram oral glucose challenge). Both IFG and IGT are considered states of pre-clinical diabetes, with over 50% of individuals with IFG progressing to frank type II diabetes within on average three years (Nichols, Diabetes Care 2007. (2): 228-233. PMID 17259486). Insulin resistance is caused, at least in part, by chronic low-grade inflammation.

Pre-clinical type II diabetes or “at risk” for type II diabetes can be defined as impaired fasting glucose, which is defined as a fasting glucose greater than 100 mg/dL. Humans with impaired fasting glucose levels and who are “at risk” of developing type II diabetes are asymptomatic.

Early-Stage Type II Diabetes.

Early-stage type II diabetes is defined by a fasting blood glucose reading of >126 mg/dL on two separate occasions. Individuals with early-stage type II diabetes do not have symptoms or signs of tissue damage or end-organ damage.

Advanced Type II Diabetes.

Advanced type II diabetes is characterized by persistent elevation in blood glucose levels over 200 mg/dL in a non-fasting state, or multiple readings of >126 mg/dL in the fasting state, and a hemoglobin A1c reading of >7%. Humans with advanced type II diabetes frequently have symptoms, microvascular complications, and/or end-organ or tissue damage.

In one embodiment, one or more metabolic and inflammatory markers are used to identify individuals at increased risk for type II diabetes whom have active inflammation, and thus are at highest risk for progression of their type II diabetes and also most likely to respond to treatment with the combination of an aminoquinoline and a statin. For example, individuals with a body mass index (BMI) greater than 25 are tested for their fasting blood glucose, hemoglobin A1c, and hs-CRP. Individuals who exhibit a fasting blood glucose >126 mg/dL on two separate occasions, and who have either a hemoglobin A1c>6.5% or a hs-CRP >3 mg/L, are identified as being at highest risk for progression of disease and initiated on therapy with the combination of HCQ and atorvastatin.

In another embodiment, one or more of these same metabolic and inflammatory markers are used to monitor an individual's response to treatment, to determine if treatment needs to be continued, or to determine if treatment can be discontinued. For example, individuals at increased risk for type II diabetes who are being treated with the combination of HCQ and atorvastatin are monitored annually, by testing for hemoglobin A1c and hs-CRP. Individuals, whose hemoglobin A1c declines to less than 5.6% and hs-CRP declines to below 1 are identified as having exhibited a positive response to therapy and that their at-risk state, early-disease state, or established disease state has responded well to treatment.

Metabolic Syndrome.

The International Diabetes Federation consensus worldwide definition of the metabolic syndrome (2006) is: Central obesity (defined as waist circumference# with ethnicity-specific values) AND any two of the following: Raised triglycerides: >150 mg/dL (1.7 mmol/L), or specific treatment for this lipid abnormality; Reduced HDL cholesterol: <40 mg/dL (1.03 mmol/L) in males, <50 mg/dL (1.29 mmol/L) in females, or specific treatment for this lipid abnormality; Raised blood pressure (BP): systolic BP>130 or diastolic BP>85 mm Hg, or treatment of previously diagnosed hypertension; Raised fasting plasma glucose (FPG): >100 mg/dL (5.6 mmol/L), or previously diagnosed type 2 diabetes. The World Health Organization 1999 criteria require the presence of any one of diabetes mellitus, impaired glucose tolerance, impaired fasting glucose or insulin resistance, AND two of the following: Blood pressure: ≧140/90 mmHg; Dyslipidemia: triglycerides (TG): ≧1.695 mmol/L and high-density lipoprotein cholesterol (HDL-C) ≦0.9 mmol/L (male), ≦1.0 mmol/L (female); Central obesity:waist:hip ratio >0.90 (male); >0.85 (female), or body mass index >30 kg/m2; and Microalbuminuria: urinary albumin excretion ratio ≧20 μg/min or albumin:creatinine ratio ≧30 mg/g. Associated diseases and signs are: hyperuricemia, fatty liver (especially in concurrent obesity) progressing to NAFLD, polycystic ovarian syndrome (in women), and acanthosis nigricans. Progression of metabolic syndrome results in frank diabetes or high fasting blood glucose or impaired glucose tolerance, and as a result individuals develop the symptoms and signs of coronary artery disease, type II diabetes, heart disease, diabetes, or other health problems.

In one embodiment, one or more metabolic and inflammatory markers are used to identify individuals at increased risk for metabolic syndrome and who have active underlying disease, and thus are at high risk for disease progression and most likely to respond to treatment with the combination of an aminoquinoline and a statin. For example, individuals with a body mass index (BMI) greater than 25 are tested for their fasting blood glucose and hemoglobin A1c, total cholesterol, LDL cholesterol, HDL cholesterol, triglycerides, and blood pressure. Individuals who have a fasting blood glucose >126 on two occasions, and at least 1 of the following, or at least 2 of the following, or at least 3 of the following, are identified as being at high-risk for development of metabolic syndrome and are initiated on treatment with the combination of atorvastatin and HCQ. Signs and findings include Blood pressure: ≧140/90 mmHg; Triglycerides (TG): ≧1.695 mmol/L and high-density lipoprotein cholesterol (HDL-C) ≦0.9 mmol/L (male), ≦1.0 mmol/L (female); or Microalbuminuria: urinary albumin excretion ratio ≧20 μg/min or albumin:creatinine ratio ≧30 mg/g.

In another embodiment, one or more of these same metabolic and inflammatory markers are used to monitor an individual's response to treatment, to determine if treatment should be continued, or to determine if treatment can be discontinued. For example, individuals at increased risk for metabolic syndrome who are being treated with the combination of HCQ and atorvastatin are monitored annually. Individuals, whose fasting blood glucose returns to less than 126 mg/dL, hemoglobin A1c declines to less than 5.6%, blood pressure becomes less than 140/90 mmHg, triglycerides (TG)<1.695 mmol/L and high-density lipoprotein cholesterol (HDL-C) increases, microalbuminuria:urinary albumin excretion ratio normalizes are identified as having exhibited a positive response to therapy, and that their at-risk state, early-disease state, or established metabolic disease state has responded well to treatment.

Determination of an Individual with “Pre-Clinical” or Early-Stage Non-Alcoholic Fatty Liver Disease and Non-Alcoholic Steatohepatitis (NASH)

Non-Alcoholic Fatty Liver Disease (NAFLD) and Non-Alcoholic Steatohepatitis (NASH).

NAFLD and non-alcoholic steatohepatitis NASH are conditions associated with fatty infiltration of the liver. Although fatty infiltration alone does not cause liver damage, when it is accompanied by an inflammatory reaction it can lead to fibrosis and liver cirrhosis and ultimately hepatic failure. The inflammation in NASH is characterized by infiltration of the liver by macrophages and lymphocytes, as well as alterations in the liver's macrophage-like Kupfer cell population (Tilg, et al, 2010. Hepatology. 52(5):1836-46). Inflammatory cytokines, particularly TNF, are central to the pathology of NASH. The source of TNF is unclear: it may be peripheral, i.e., inflammatory adipose tissue, or local, i.e., innate immune cells activated by portal-derived endotoxin or by free fatty acid (Tilg et al, 2010. Hepatology. 52(5):1836-46). The endotoxin-responsive TLR4 receptor has been shown to be critical to disease in a mouse model of NASH (Tsukumo et al, Diabetes 2007. 56(8):1986-98).

Pre-clinical NASH or “at risk” for NASH can be defined as NAFLD, which is the presence of fatty infiltration of the liver in the absence of alcohol consumption or exposure to other liver toxins. Humans with NAFLD and who have pre-clinical NASH (i.e., NAFLD) have normal levels of liver enzymes in their blood (e.g. normal aminotransferase [transaminase] levels, including a normal AST (SGOT) and ALT (SGPT)).

Early-Stage NASH.

Early-stage NASH is defined as the presence of NAFLD in conjunction with hepatic inflammation and injury, as reflected by abnormally high levels of blood aminotransferases (i.e., elevated levels of AST (SGOT) and ALT (SGPT) as compared to the normal range in humans).

Advanced NASH.

Advanced NASH is defined as the presence of chronic liver inflammation and injury, as reflected by persistently elevated levels of liver transaminases (persistently elevated AST (SGOT) and ALT (SGPT)), and the presence of early or advanced hepatic fibrosis and/or cirrhosis. Hepatic fibrosis is identified by ultrasound or CT or MRI imaging of the liver, or by liver biopsy.

In one embodiment, one or more metabolic markers, inflammatory markers and imaging markers are used to identify individuals at increased risk for NAFLD or NASH, and thus are most likely to respond to treatment with the combination of an aminoquinoline and a statin. For example, individuals with elevated liver transaminases, based on AST >60 IL/L (normal range 6-40 IU/L) or ALT >50 IU/L (normal range 7-35 IU/L), ultrasound findings indicative of fatty liver, and a fasting blood glucose >126 on two separate readings, or hemoglobin A1c >6.5%, are identified as being at high risk for progression to NASH and initiated on therapy with the combination of HCQ and atorvastatin.

In another embodiment, one or more of these same metabolic and inflammatory markers are used to monitor an individual's response to treatment, to determine if the individual has exhibited a positive response to therapy, or to determine if treatment can be discontinued. For example, individuals at increased risk for NAFLD or NASH who are being treated with the combination of HCQ and atorvastatin are monitored annually, by testing for AST, ALT, hemoglobin A1c and fasting blood glucose. Individuals, whose AST and ALT normalize, whose hemoglobin A1c declines to less than 5.6%, and whose fasting blood glucose normalizes are identified as having exhibited a positive response to therapy, and that their at-risk state, early-disease state, or established disease state has responded well to treatment.

### Assessing Inflammation in Pre-Clinical Disease, Early-Stage Disease, and Established Disease

The following provides examples of approaches to determining whether inflammation is present in an individual, including individuals at risk for a variety of different inflammatory diseases, such as autoimmune diseases (e.g., RA, MS, Crohn's disease, psoriasis, etc), degenerative diseases involving low-grade inflammation (e.g., OA, Alzheimer's disease, macular degeneration, etc), other inflammatory diseases (e.g., NASH, type II diabetes, metabolic syndrome, atherosclerosis, cardiac disease, etc.), as well as inflammatory diseases associated with chronic inflammation (e.g., HIV infection, HCV infection, CMV infection, TB infection, etc). Although the following describes the approach to identifying inflammation particularly in humans at risk of developing arthritis or with early-stage arthritis, in another embodiment it is use to assess disease activity or tissue or organ damage in individuals with established inflammatory disease.

A variety of markers can be used to assess inflammation in inflammatory diseases, including imaging markers, molecular markers, and clinical markers. Detection of such markers can facilitate identification of individuals with pre-clinical and early-stage inflammatory disease, and can be used to assess the level of disease activity and ongoing tissue damage in established disease.

A comprehensive description of the markers for OA and RA are presented as an example of how one approaches developing markers for a pre-clinical or early-stage inflammatory disease in general. In arthritis, examples of clinical markers include warmth, erythema (redness), inflammation, and effusions. Other examples of clinical markers are morning stiffness in the joint lasting more than 1 hour, and pain and swelling. Examples of imaging markers include MRI- or ultrasound-detected inflammation in the joint. MRI, performed with or without gadolinium contrast, detects inflammation on the basis of the presence of one or more of the following findings: synovitis (synovial lining thickening, proliferation and/or enhancement (increased signal on Gd-MRI)); increased Doppler-flow signal in the synovial lining); a joint effusion; extensive bone marrow edema; and other findings suggestive of inflammation. When ultrasound is the imaging method used, inflammation is defined by the presence of one or more of the following findings: synovial lining thickening and/or enhancement, a joint effusion, bone marrow enhancement, and other findings suggestive of inflammation. Molecular markers that can be used in assessing inflammation include ESR, CRP, cytokines, chemokines, and other inflammatory mediators. ESR and CRP are measured in blood, and the other molecular markers of inflammation can be measured in blood or synovial fluid. Use of molecular markers in blood for identifying individuals with pre-clinical RA or early-stage RA is described in Sokolove et al. (PLoS One. 2012; 7(5):e35296) and Deane et al. (Arthritis Rheum. 2010 62(11):3161-72.).

The presence of pre-clinical and early-stage inflammatory disease may be determined or confirmed by a difference in level of a molecular and inflammatory markers in body fluids, including without limitation synovial fluid, or joint tissue relative to that in a control body fluid or joint tissue that is free of arthritis. Examples of such changes in levels of molecular markers in pre-clinical and early-stage OA and RA are the following: increase in level of interleukin-1 beta (IL-1β); increase in level of TNF; increase in ratio of IL-1β to IL-1 receptor antagonist protein (IRAP); increase in expression of p55 TNF receptors (p55 TNF-R); increase in level of interleukin-6 (IL-6); increase in level of leukemia inhibitory factor (LIF); altered levels of insulin-like growth factor-1 (IGF-1), increase in levels of transforming growth factor beta (TGFβ), platelet-derived growth factor (PDGF), or basic fibroblast growth factor (b-FGF); increase in level of keratan sulfate; increase in level of stromelysin; increase in ratio of stromelysin to tissue inhibitor of metalloproteases (TIMP); increase in in level of osteocalcin; increased alkaline phosphatase; increased cAMP responsive to hormone challenge; increased urokinase plasminogen activator (uPA); increase in level of cartilage oligomeric matrix protein; increase in level of collagenase; increase in level of other cytokines; increase in in level of CRP; or increase in in level of autoantibodies against synovial joint proteins or other biomolecules. The term “metalloprotease” as used herein is intended to refer to MMPs, especially those whose levels are typically elevated concentrations where there is articular cartilage degeneration, i.e., stromelysins, collagenases, and gelatinases. Aggrecanase is also included within this term. The three collagenases present in articular cartilage during the early stages of degeneration are collagenase-1 (MMP-1), collagenase-2 (MMP-8), and collagenase-3 (MMP-13). Of the three stromelysins, stromelysin-1 (MMP-3), stromelysin-2 (MMP-10), and stromelysin-3 (MMP-11), only stromelysin-1 appears in articular cartilage during the early stages of its degeneration. The metalloproteases are secreted by chondrocytes as proenzymes, which must be activated before they can degrade extracellular matrix macromolecules. Activation of these proenzymes involves an enzymatic cascade in which serine proteases, including the plasminogen activator-plasmin system, play a key role. This example is provided for OA and RA, but the approach, the types of markers, and a subset of the markers are relevant for a wide spectrum of inflammatory diseases.

IL-1, which exists as IL-1α and IL-1β, is a catabolic cytokine that mediates articular cartilage injury and loss in mammalian joints. It suppresses the synthesis of type II collagen found in articular cartilage, while promoting the synthesis of type I collagen characteristic of fibroblasts; induces the production of enzymes involved in matrix degradation; and suppresses the ability of chondrocytes to synthesize new proteoglycans. IL-1 and its modulator IRAP are produced in an autocrine and paracrine fashion by synovial macrophages, and IRAP production may increase in the presence of granulocyte macrophage colony-stimulating factor (GM-CSF). However, IL-1 is much more potent than IRAP, with approximately 130-fold more IRAP being required to abolish the pathogenic effects of IL-1, as measured in chondrocytes and cartilage explants. Imablances between IL-1 and IRAP exacerbates the degeneration of articular cartilage. Consequently, it is also appropriate to identify abnormalities in the levels of IL-1 and IRAP, as well as in the ratio of IL-1 to IRAP, to identify an individual in the early stages of cartilage injury and loss before focal cartilage loss can be identified radiographically. Thus, determining the levels of IL-1 and IRAP, as well as the ratio of IL-1 to IRAP, could enable identification of individuals that are candidates for early pharmacological intervention before significant cartilage degeneration occurs. Furthermore, the frequency of IL-1α- and IL-1β-secreting macrophages is significantly greater in the synovial fluid and synovial tissue of joints undergoing the early stages of articular cartilage degeneration can be detected and is significantly greater than in synovial fluid and synovial tissue from normal joints, i.e., joints in which there is no articular cartilage degeneration.

In mammals subjected to sectioning of the cruciate ligament of a knee joint, the concentration of TNF is significantly higher in the synovial fluid of the sectioned knee joint than in that of the contralateral, unsectioned knee joint. The expression of p55 TNF receptors (TNF-R) on chondrocytes in articular cartilage is also higher in the sectioned knee joint. Therefore, because an increase in TNF levels, and possibly TNF signaling, is associated with early cartilage injury and loss, it is appropriate to measure levels of TNF and TNF-R in the joints of individuals at risk for cartilage degeneration and loss. These results contribute to diagnostic classification of individuals that are candidates for early pharmacological intervention before significant cartilage degeneration occurs.

IL-6 is an inflammatory cytokine whose are abnormally high in the joints and synovial fluid of damaged limbs. IL-6 increases the expression of TNF-R on chondrocytes and the production of proteoglycan by chondrocytes; it also induces the release of glycosaminoglycans from the cartilage matrix. Comparing IL-6 levels in synovial fluid and chondrocytes of joints in the early stages of articular cartilage injury and loss to that in synovial fluid and chondrocytes of control joints can identify individuals that are appropriate candidates for pharmacological treatment, before any focal cartilage loss is detectable by radiographic examination.

LIF is produced by monocytes, granulocytes, T cells, fibroblasts, and other cell types associated with inflammatory conditions. Synoviocytes and chondrocytes synthesize and secrete LIF in the presence of IL-1β and TNFα. Thus, identifying increases in levels of LIF can allow selection of candidates for pharmacologic treatment of the early stages of articular cartilage injury and loss.

IGF exists as types I and II, and IGF-I mediates cartilage synthesis. Furthermore, it reduces degradation and promotes synthesis of proteoglycans even in the presence of IL-1β and TNFα. Serum levels of IGF-1 are maintained by high-affinity binding proteins (IGF-BPs), and IGF-1 regulates both bone and cartilage turnover. Detecting abnormally high levels of IGF-1 permits identification of candidates for early pharmacologic treatment of articular cartilage degeneration.

TGFβ is produced by chondrocytes and acts as a powerful mitogen contributing to the turnover of both cartilage and bone. Further, it stimulates the synthesis of extracellular matrix and has anti-inflammatory activity. It also inhibits the degradation of the extracellular matrix by stimulating the production of protease inhibitor, and blocking the release of collagenases and metalloproteases. Further still, it promotes cartilage repair by stimulating production of collagen, fibronectin, inhibitors of plasminogen activators, and tissue inhibitors of metalloproteases (TIMP) by various cells in the mammal joint. Synovial fluid levels of TGFβ are abnormally low in the joints of mammals in the early stages of articular cartilage injury and loss. Consequently, levels of TGFβ compared to control permit diagnostic evaluation of candidates for early pharmacologic treatment of articular cartilage degeneration.

With progressive degeneration, i.e., catabolism of the articular cartilage in the joint, a number of metabolites are produced that are useful as markers of the cartilage degeneration, both to the occurrence and to the progression of cartilage degeneration. For example, IL-1α and IL-1β or TNFα active inflammatory and degradative pathways that mediate cartilage degradation and release of glycosaminoglycans (GAGS), which can be measured in the synovial fluid of an individual. Furthermore, GAG levels change after treatment so that it is possible to monitor the efficacy of pharmacologic intervention, by using GAG levels in synovial fluid as a marker of articular cartilage turnover. Because the degradation of articular cartilage involves collagen as well as the other cartilage components, several collagen breakdown products serve as markers of cartilage degradation in mammals. Type-II-specific collagen breakdown products, e.g., 20-30 amino acid neoepitopes, can be identified in body fluids such as synovial fluid, plasma, serum, and urine. The presence of collagen neoepitopes in these body fluids may be used as indicators of OA onset and progression.

The presence or an increase in the levels of 5D4, a neo-epitope of the GAG keratan sulfate, in synovial fluid is a marker of early articular cartilage injury and loss. Conversely, presence of or increased levels of various neo-epitopes of chondroitin sulfate, another GAG, is associated with anabolic events in the articular cartilage of mammals in the early stages of cartilage injury and loss. Levels of these epitopes in synovial fluid, particularly 3B3, 7D4 and 846, can be determined by specific monoclonal antibodies. The 3B3 epitope is expressed on chondroitin sulfate chains of cartilage during repair and remodeling of the extracellular matrix, and consequently its levels in synovial fluid correlate inversely with those of 5D4. The determination of 3B3 levels in the synovial fluid of test mammals and comparison of these levels with control values permits the creation of a diagnostic profile of a mammal that is an appropriate candidate for early pharmacologic treatment.

Additional markers of cartilage anabolism are the propeptides of type II procollagen (PIIP). Type II collagen is the major collagen of articular cartilage and is produced by chondrocytes as the procollagen PIIP. During the process of collagen fibril formation, aminopropeptide and carboxypropeptide, the noncollagenous portions of PIIP, are cleaved and released into body fluids, where they can be measured as a reflection of anabolic activity in articular cartilage. Levels of the carboxypropeptide of PIIP (carboxy-PIIP) in synovial fluid are higher during cartilage anabolism and correlate with radiographic evidence of pathologic changes in the cartilage. Accordingly, detection of increased levels of carboxy-PIIP in synovial fluid identifies individual for early pharmacologic treatment.

Perturbation of the stromelysin:TIMP ratio in articular cartilage and joint fluids of mammals is another marker of early-stage articular cartilage degeneration. Abnormal joint loading after joint injury causes the production of excess stromelysin, an enzyme produced by chondrocytes and synoviocytes in an IL-1-mediated process. The concentrations of stromelysin are higher in fibrillated (injured) cartilage than they are in cartilage more distal to the injury. Levels of stromelysin may be excessively high for only a short period of time, but where the damage to the joint transcends the tidemark zone of the articular cartilage and reaches into the subchondral bone, there is a substantial likelihood of subsequent articular cartilage degeneration, usually preceded by a stiffening of the subchondral bone. In such situations, there is an increased number of cells involved in the synthesis of stromelysin, IL-1α, IL-1β, and the oncogene proteins c-MYC, c-FOS, and c-JUN. In the synovium cells that secrete these factors are the superficial synovial lining cells, while in the cartilage such cells are the chondrocytes in the superficial and middle layers and the condrocytes in the fibrillated areas of the tibial plateau. Further, stromelysin and IL-1 diffuse into the cartilage matrix of the tibial plateau. Stromelysin, which degrades components of connective tissue, including proteoglycans and type IX collagen, is actively synthesized in the synovium of mammals in the early stages of articular cartilage degeneration, and is the primary proteolytic enzyme involved in the cartilage destruction. Increased levels of stromelysin mRNA are detectable in the synovia of such mammals, as are increased levels of collagenase mRNA. Increased levels of both isoforms of IL-1, but especially IL-1β, stimulate the synthesis of stromelysin and collagenase by synovial fibroblasts. IL-1 does not stimulate the production of tissue inhibitor of metalloprotease (TIMP), such that the levels of this metalloprotease inhibitor in the synovium remain unchanged while the levels of metalloproteases are dramatically increased. The above text represents a detailed description is for OA and RA, but the approach, the types of markers, and a subset of the markers are relevant for a wide spectrum of inflammatory diseases, and these descriptions are meant to serve as an example of how one approaches developing markers for a pre-clinical or early-stage inflammatory diseases in general.

### Assessment of Biomarkers for Determination of an Individual Eligible for Treatment

In some embodiments the methods of the invention comprise the step of determining the presence of early-stage inflammatory disease in an individual or susceptibility to development of inflammatory disease prior to treatment, and thus indicating a need of treatment. The method may further include determining the presence of inflammation, prior to the administering step, where an individual at increased risk or in an early stage of an inflammatory disease showing signs of inflammation, particularly inflammation of the relevant organ is selected for treatment with the combination therapy of the invention. The biomarkers relevant to each disease are presented in the descriptions of each of the diseases. Such biomarkers include clinical biomarkers, metabolic biomarkers, inflammatory biomarkers, imaging biomarkers, research biomarkers, and other biomarkers, with distinct subsets of biomarkers being relevant for different diseases.

In some embodiments the treatment with a combination of hydroxychloroquine and atorvastatin prevents the development of disease. In some embodiments the treatment with a combination of hydroxychloroquine and atorvastatin prevents the progression of signs or symptoms of an inflammatory disease. In some embodiments the treatment with a combination of hydroxychloroquine and atorvastatin results in the early signs or symptoms of an inflammatory disease returning to normal. In some embodiments, treatment with a combination of hydroxychloroquine and atorvastatin results in normalization of inflammatory markers. In some embodiments the treatment with a combination of hydroxychloroquine and atorvastatin prevents development of organ or tissue damage. In some embodiments the treatment with a combination of hydroxychloroquine and atorvastatin results in stabilization or normalization of laboratory test, imaging markers, or other markers of disease.

In yet other embodiments, the treatment with a combination of hydroxychloroquine and atorvastatin is used to treat established disease in an individual exhibiting elevated inflammatory markers. In some embodiments, treatment of established inflammatory disease with a combination of hydroxychloroquine and atorvastatin results in normalization of inflammatory markers and other disease markers. In some embodiments the treatment with a combination of hydroxychloroquine and atorvastatin results in stabilization or normalization of laboratory test, imaging markers, or other markers of the disease. In some embodiments the treatment with a combination of hydroxychloroquine and atorvastatin prevents development of organ or tissue damage.

Various techniques and reagents can be used in the analysis of inflammatory biomarkers in the present invention. In one embodiment of the invention, blood or synovial fluid samples, or samples derived from blood, e.g. plasma, serum, etc., are assayed for the presence of specific biomarkers. Other sources of samples are body fluids such as synovial fluid, lymph, cerebrospinal fluid, bronchial aspirates, saliva, milk, urine, and the like. Also included are derivatives and fractions of such cells and fluids. Diagnostic samples are collected any time that an individual is suspected of having an inflammatory disease or of being at risk of developing an inflammatory disease. Such assays come in many different formats, including autoantigen arrays; enzyme-linked immunosorbent assays (ELISA) and radioimmunoassays (RIA); assays in which binding of labeled peptides in suspension or solution are measured by flow cytometry or mass spectrometry.

Many such methods are known to one of skill in the art, including ELISA, fluorescence immunoassays, protein arrays, eTag system, bead-based systems, tag or other array-based systems, surface plasmon resonance (SPR)-based detection systems, etc. Examples of such methods are set forth in the art, including, inter alia, chip-based capillary electrophoresis: Colyer et al. (1997) J Chromatogr A. 781(1-2):271-6; mass spectroscopy: Petricoin et al. (2002) Lancet 359: 572-77; eTag systems: Chan-Hui et al. (2004) Clinical Immunology 111:162-174; microparticle-enhanced nephelometric immunoassay: Montagne et al. (1992) Eur J Clin Chem Clin Biochem. 30(4):217-22; the Luminex XMAP bead-array system (www.luminexcorp.com); and the like, each of which are herein incorporated by reference.

For multiplex analysis, arrays containing one or more detection antibodies that recognize biomarkers of interest can be generated. Various immunoassays designed to quantitate the biomarkers may be used in screening. Measuring the concentration of the target protein or other biomarker in a sample or fraction thereof may be accomplished by a variety of specific assays. For example, a conventional sandwich-type assay may be used in an array, ELISA, RIA, bead array, etc. format.

Analysis of a biological sample may be done by using any convenient protocol, for example as described below. The readout may be a mean, average, median or the variance or other statistically or mathematically derived value associated with the measurement. The readout information may be further refined by direct comparison with the corresponding reference or control readout.

Following quantitation of the biomarker in the sample being assayed, the value obtained is compared with a reference or control value to make a diagnosis regarding the phenotype of the patient from whom the sample was obtained. Typically a comparison is made with the analogous value obtained from a sample or set of samples from an unaffected individual. Additionally, a reference or control value may be a value that is obtained from a sample of a patient known to have an autoimmune or degenerative disease of interest, such as RA or OA, and therefore may be a positive reference or control profile.

For prognostic purposes, an algorithm can be used that combines the results of determinations of multiple antibody specificities and/or cytokine levels, and/or levels of cartilage degeneration markers, and/or other markers, and that will discriminate robustly between individuals with autoimmune disease, e.g. RA, or degenerative disease, e.g. OA, and controls.

Included as a biomarker of inflammation and providing utility as a biomarker in a variety of inflammatory diseases is C reactive protein (CRP), including high-sensitivity CRP (hs-CRP). It is known that individuals with high levels of hs-CRP, even at the high end of the normal range, have 1.5 to 4 times increased risk of developing an inflammatory disease, including but not limited to atherosclerotic disease, atherosclerotic cardiovascular disease, RA, psoriatic arthritis, systemic lupus erythematosus, osteoarthritis, type II diabetes, metabolic syndrome, NAFLD, NASH and other inflammatory metabolic diseases. The American Heart Association and U.S. Centers for Disease Control and Prevention have defined risk groups based on hs-CRP levels as follows:
Low risk: hs-CRP less than 1.0 mg/L
Average risk: hs-CRP 1.0 to 3.0 mg/L
High risk: hs-CRP above 3.0 mg/L

The range of levels of plasma fibrinogen that is deemed normal varies from laboratory to laboratory but is typically 1.5-4.0 g/L. Levels of plasma fibrinogen above 2.8 g/L are associated with increased risk of developing an inflammatory disease, and levels >4 g/L are associated with an even higher risk.

Normal levels of serum amyloid A (SAA) range widely. However, elevations in SAA levels have been associated with increased risk with moderate elevation >3.9 but <8 mg/L.) conferring increase risk over the lowest tercile and values greater than 8.2 mg/L (highest tercile) imparting highest risk.

There is a wide range in ESR values that are considered normal, but ESR values suggestive of inflammation include >15 mm/hr in men under 50 years old, >20 in men over 50 and women under 50, and >30 mm/hr in women over 50.

MRI, with or without gadolinium or other contrast enhancement, can be used to detect the presence of inflammation and thereby identify individuals with an inflammatory disease or at increased risk of developing an inflammatory disease. For example, MRI-detected inflammation is defined by the presence of one or more of the following findings: synovitis (synovial lining thickening, proliferation and/or enhancement), a joint effusion, bone marrow edema, and other MRI imaging findings suggestive of inflammation (Krasnokutsky et al, Arthritis Rheum. 2011 63(10):2983-91. doi: 10.1002/art.30471 PMID: 21647860; Roemer et al, Osteoarthritis Cartilage. 2010 October; 18(10):1269-74. PMID: 20691796; Guermazi et al, Ann Rheum Dis. 2011 70(5):805-11, PMID: 21187293). Guermazi et al. (Guermazi et al, Ann Rheum Dis. 2011 70(5):805-11, PMID: 21187293) defines a semiquantiative scoring system for grading the level of inflammation in joints, allowing one to determine (1) whether an individual has inflammation or not, and (2) the degree of inflammation in an individual. Individuals with evidence of joint inflammation according to the Guermazi scoring system can be classified as having increased risk for the development of OA, pre-clinical OA, early-stage OA, or established OA. The degree of inflammation as evaluated by the Guermazi scoring system predicts development and/or progression of the inflammatory disease OA. MRI, with or without gadolinium, can be applied to many other conditions to determine whether or not inflammation is present, and whether an individual with inflammation has pre-clinical inflammatory disease, early-stage inflammatory disease, or established inflammatory disease.

Ultrasound-detected inflammation is defined by the presence of one or more of the following findings: synovial lining thickening and/or enhancement, a joint effusion, bone marrow enhancement, a Doppler-flow signal in the synovial lining, and other findings suggestive of inflammation (Guermazi et al, Curr Opin Rheumatol. 2011 23(5):484-91. PMID: 21760511; Hayashi et al, Osteoarthritis Cartilage. 2012 March; 20(3):207-14. PMID: 22266236; Haugen et al, Arthritis Res Ther. 2011; 13(6):248. PMID: 22189142).

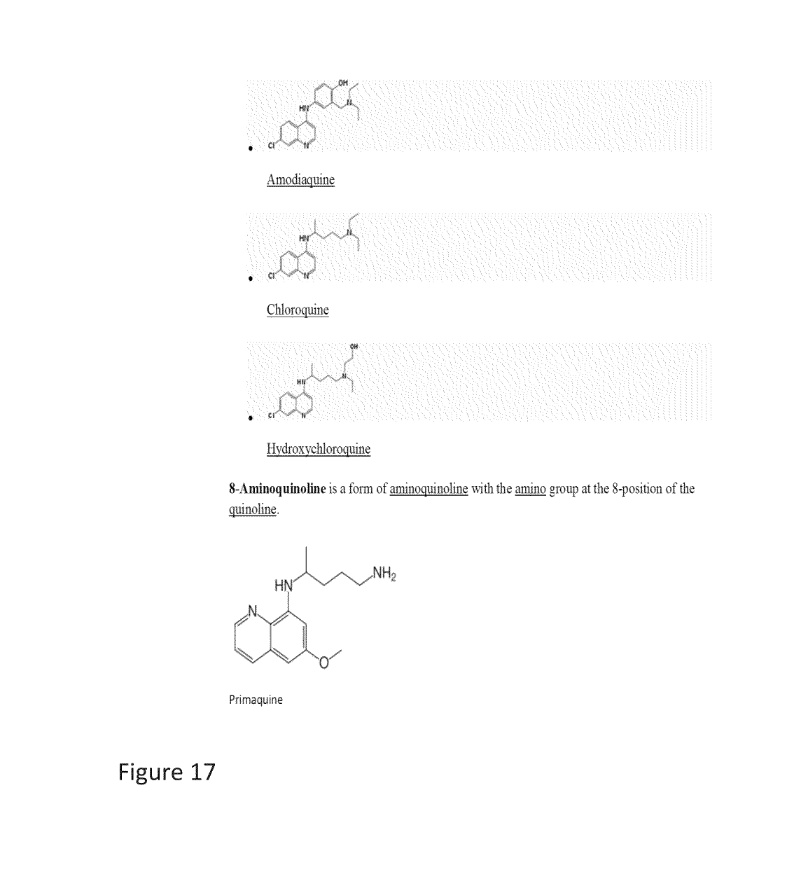

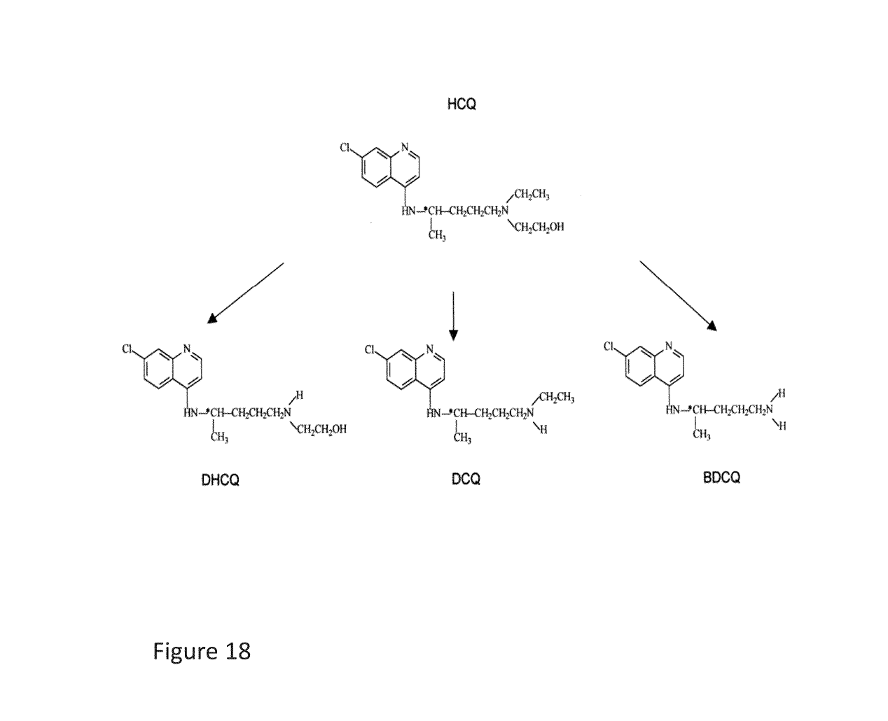

This invention relates to the use of an aminoquinoline in combination with a statin to treat inflammatory diseases. The aminoquinoline can comprise HCQ, DHCQ, or another aminoquinoline (FIGS. 17 and 18). In one embodiment the statin comprises atorvastatin, and in other embodiments the statin can comprise cerivastatin, fluvastatin, lovastatin, mevastain, or pitavastatin. Importantly, this novel use of a combination of an aminoquinoline and a statin does not require use of an antibiotic, an anti-viral, or an anti-bacterial agent. No antibiotic, anti-viral, or anti-bacterial compound is needed for the anti-inflammatory activity and disease-modifying activity described for the combination of an aminoquinoline and a statin.

In certain in vitro assays, ex vivo assays, and in vivo models, the combination of an aminoquinoline and a statin, with the preferred embodiment comprising atorvastatin in combination with HCQ or DHCQ, exhibits unexpected and surprising synergy in reducing the production of inflammatory mediators in in vitro and ex vivo assays, and in reducing disease activity and inflammation in in vivo models. In other in vitro assays, ex vivo assays, and in vivo models, the combination exhibits an unexpected and surprising additive effect in reducing the production of inflammatory mediators in in vitro and ex vivo assays, and reducing disease activity and inflammation in the in vivo model. In general, the individual aminoquinoline and individual statin alone, including use of atorvastatin alone, HCQ alone, or DHCQ alone, did not provide as robust anti-inflammatory or disease-modifying activity as did the combinations (the combination of HCQ+atorvastatin, or the combination of DHCQ+atorvastatin), which can provide for a synergistic benefit when combined.

### Use of Biomarkers to Guide Treatment

Multiple markers of inflammatory disease can be used to identify individuals at increased risk for disease, with early-stage disease, as well as to monitor response to intervention with HCQ and atorvastatin therapy. Such markers, termed biomarkers, including laboratory test results, imaging results, physical findings, research test markers, and other markers of inflammation and disease. Examples of laboratory markers include: hs-CRP as a measure of systemic inflammation; ESR as a measure of systemic inflammation; hemoglobin A1C as a measure of poor glucose control and thus the severity of diabetes and/or metabolic syndrome; liver enzyme tests as a measure of hepatic dysfunction and of the activity of NAFLD or NASH; and cholesterol and LDL cholesterol as a sign of atherosclerosis. Examples of imaging markers include, evidence of early synovitis on MRI of the hand joints in individuals with pre-clinical or early stage RA; evidence of low-grade synovitis on MRI of a joint in individuals at-risk for OA; evidence of demyelinating lesions on MRI of the brain of an individual at risk for MS. Examples of research biomarkers include: multiplex profiling of cytokines in blood to identify individuals with systemic inflammation, and to determine the specific subset of cytokines causing the individual to be “at-risk” or mediating early-stage disease; analysis of gene expression to subtype the inflammatory disease; analysis of genetic variants through genotyping or sequencing the genome of an individual to determine which inflammatory disease(s) an individual is at increased risk for developing. In other embodiments, such laboratory, imaging and research biomarkers are used to identify individuals at increased risk for developing, or with early-stage, inflammatory disease. In other embodiments, such laboratory, imaging and research biomarkers are using to monitor an individual's response to combination therapy with HCQ and atorvastatin therapy, to determine if therapy need to be continued, or if therapy needs to be increased, or if an individuals' risk has decreased and thus therapy can be discontinued.

### EXAMPLES

The following are examples of the methods and compositions of the invention. It is understood that various other embodiments may be practiced, given the general description provided above.

### Example 1

### Treatment with the Combination of HCQ and Atorvastatin Inhibited the Development of and Reduced the Severity of Mouse Osteoarthritis (OA)

Mouse Models of OA.

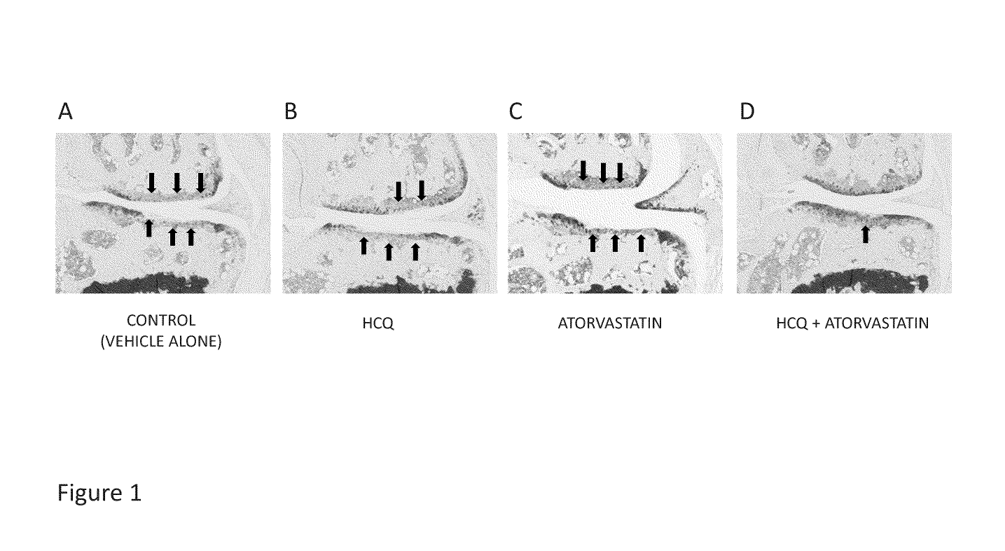

C57BL6 (B6) mice (n=7-10 per group) were surgically induced to develop OA by medial meniscectomy (MM) or destabilization of the medial meniscus (DMM). Experiments were performed under protocols approved by the Stanford University Committee of Animal Research and in accordance with NIH guidelines. Mouse OA was generated either by DMM (Glasson, S., S., et al., Osteoarthritis Cartilage, 15: 1061-1069 (2007)) or by MM (Kamekura, S., et al., Osteoarthritis Cartilage, 13: 632-641 (2005)). One week and two weeks following surgical induction of the MM or DMM model, the articular cartilage is intact and there is no overt evidence of OA—at this time point the mice walk and run normally and are asymptomatic or can exhibit mild joint symptoms, but owing to the surgical procedure the mice have pre-clinical or early-stage OA and go on to develop established OA over the following months (FIG. 1A).

Histological Scoring of Mouse OA.

Mice were euthanized 13 weeks after surgery and 12 weeks after the initiation of treatment. Their stifle joints were decalcified in EDTA solution, fixed in 15% formalin, and embedded in paraffin. Serial 4-μm sections were cut and stained with safranin-O, Scoring of arthritis in these histology sections was done according to a modified version of previously described composite scoring systems (Kamekura, S., et al. Osteoarthritis Cartilage 13: 632-641 (2005); Bendele, A. M., J Musculoskelet Neuronal Interact., 1: 363-376 (2001)). The “Cartilage Degeneration Score” (also termed the “OA Score” or “Histologic Score”) was calculated as follows: cartilage degeneration (0-4) was multiplied by the width (1=1/3, 2=2/3, and 3=3/3 of surface area) of each third of the femoral-medial and tibial-medial condyles, and the scores for the 6 regions were summed. To evaluate osteophyte formation, we scored toluidine-blue-stained sections according to a previously described scoring system (Kamekura, S., et al. Osteoarthritis development in novel experimental mouse models induced by knee joint instability. Osteoarthritis Cartilage 13, 632-641 (2005)): 0, none; 1, formation of cartilage-like tissues; 2, increase of cartilaginous matrix; 3, endochondral ossification. To evaluate synovitis, we scored H&E-stained sections according to a previously described scoring system (Blom, A. B., et al. Synovial lining macrophages mediate osteophyte formation during experimental osteoarthritis. Osteoarthritis Cartilage 12, 627-635 (2004)): 0, no changes compared to normal joints; 1, thickening of the synovial lining and minimal influx of inflammatory cells; 2, thickening of the synovial lining and moderate influx of inflammatory cells; and 3, profound thickening of the synovial lining (more than four cell layers) and maximal observed influx of inflammatory cells. Scores for osteophyte formation and synovitis (inflammation in the synovial lining and joint) were recorded for the femoral-medial and the tibial-medial condyles on the operated side of the joint, and the scores for the two regions were summed and statistical comparisons performed using the t test.

Drug Dosing.

Treatment was started 1 week after DMM, a time at which mice were in the pre-clinical stage of OA, i.e., they do not have classic histologic features of OA, such as overt cartilage loss or bone remodeling (osteophyte formation, subchondral bone remodeling), but they may have cartilage edema, proteoglycan loss, and other features characteristic of pre-clinical OA in both mice and humans. DMM in mice resembles a degenerative or traumatic meniscal tear in humans, which has been demonstrated to increase the risk of developing OA by approximately 5-fold.

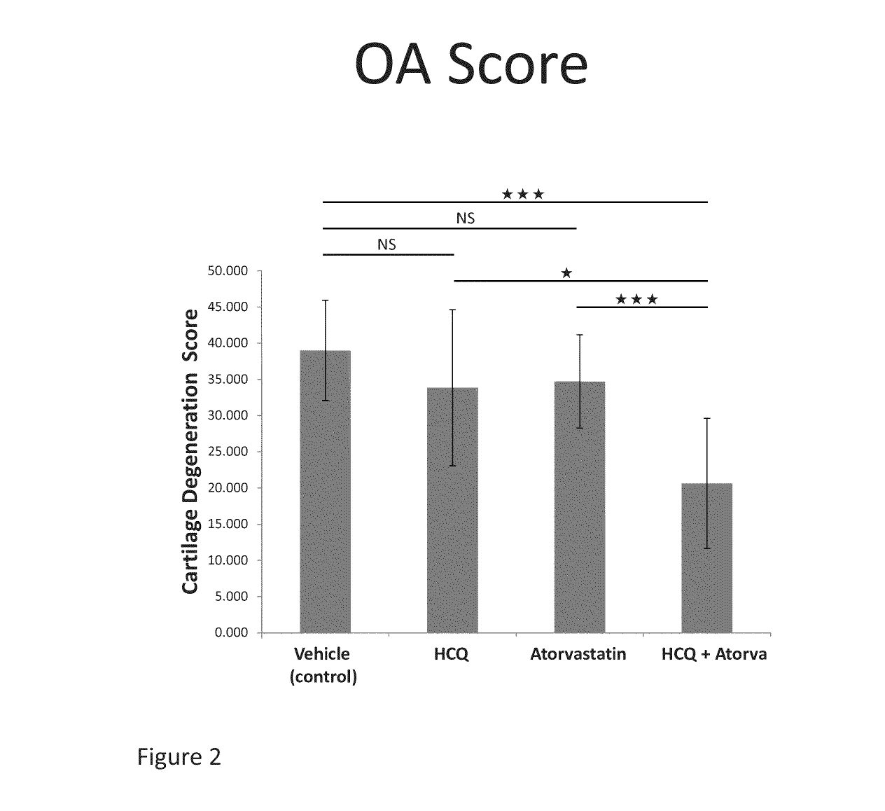

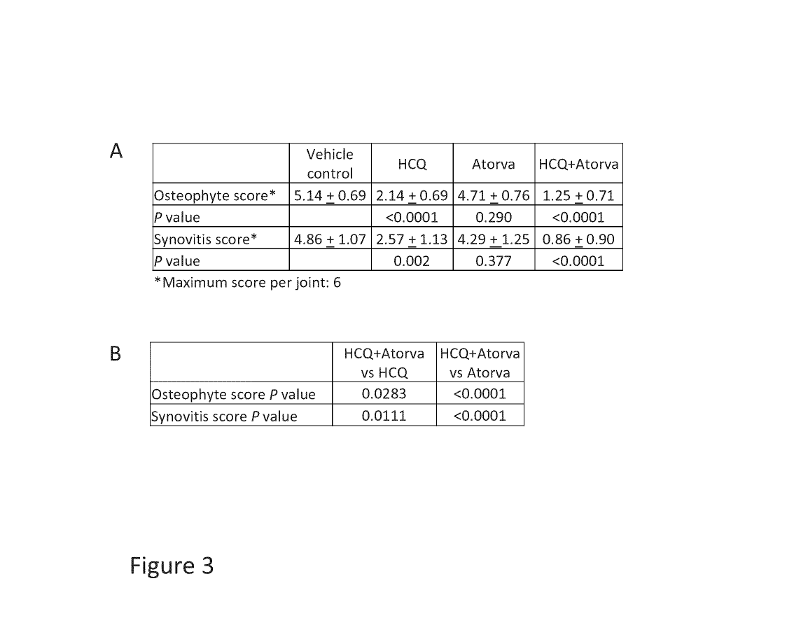

One week after undergoing DMM, mice (7-10 per treatment arm) were administered HCQ sulfate 100 mg/kg/day alone, atorvastatin calcium 40 mg/kg/day alone, or a combination of HCQ sulfate 100 mg/kg/day plus atorvastatin calcium 40 mg/kg/day by oral gavage in 100-ul volumes once per day. Mice in the control groups were treated with vehicle alone. 12 weeks later, mice were sacrificed, their joints harvested, joint sections cut, and tissue sections stained with safranin-O or with hematoxylin and eosin (H&E). An examiner blinded to treatment used microscopy to score the severity of OA based on “OA scores” representing the severity of cartilage degeneration. The degree of synovitis and osteophyte formation was independently scored by a blinded examiner. The combination of HCQ and atorvastatin significantly reduced the severity of OA CP<0.05, by t test), whereas treatment with HCQ alone or atorvastatin alone did not (FIG. 2). In addition, the combination of HCQ and atorvastatin significantly reduced the development of osteophytes and synovitis (inflammation) associated with OA in the DMM model (P<0.001, by t test) (FIG. 3).

Thus, we demonstrate that in the DMM mouse model of OA a combination of HCQ plus atorvastatin prevented the development of OA from its earliest pre-clinical phase, reduced the severity of OA, and reduced joint inflammation.

### Example 2

### The Combination of Hydroxychloroquine and Atorvastatin Reduced Synovitis and Improved the Pain and Functional Scores in Humans with Medial-Compartment Knee OA in a 16-Week, Open-Label, Pilot Clinical Trial

Nearly 27 million people in the U.S. have some form of OA, a number that has increased from 21 million in 1990. Knee OA is prevalent in 16% of all adults 45 years and older. In Canada, OA affects 10% of the entire population. In 2005, the cost of loss of productivity by U.S. workers as a result of OA exceeded $70 billion. Medical therapies used to treat OA include NSAIDs, acetaminophen, intra-articular corticosteroids, intra-articular hyaluronic acid formulations, narcotics, and physical therapy. While all of these treatments may alleviate the symptoms of OA, there are no medical therapies currently available that prevent the progression of cartilage loss or reverse the disease process. In patients with more severe knee OA, total joint replacement is an option. The incidence of total knee replacement is steadily rising, and OA is the leading cause of knee replacement surgery. The increased incidence of knee replacement surgery is putting a burden on the healthcare system as well as creating a risk for surgical complications. A population-based study showed that the incidence of total knee replacements in patients over 45 years of age increased by 81.5% between 1990 and 2000. The total cost of total knee replacements to the US healthcare system in 2000 was approximately $148 million.

Our preclinical studies demonstrated that the combination of hydroxychloroquine and atorvastatin prevents the development of OA in the destabilization of the medial meniscus (DMM) mouse model (FIGS. 1-3). The combination of hydroxychloroquine and atorvastatin provided statistically significant benefit in this model, whereas several other drug combinations, or treatment with HCQ alone or atorvastatin alone, did not.

To date, HCQ has been tested in general human OA (e.g. the non-erosive common type of OA) but has failed to demonstrate disease-modifying or pain-reducing activity in non-erosive OA.

To determine whether the combination of hyroxychloroquine and atorvastatin could provide benefit in humans with established OA, we initiated pilot clinical trial in human with medial compartment OA of the knee. This trial was entitled “Hydroxychloroquine/Atorvastatin in the Treatment of Osteoarthritis (OA) of the Knee” and was registered and assigned the ClinicalTrials.gov Identifier: NCT01645176. A primary objective of the clinical trial was to evaluate the efficacy of the combination of hydroxychloroquine and atorvastatin in treating established medial compartment knee OA (non-erosive) by assessing changes in synovitis of the knee between 0 and 24 weeks as measured by MRI. Secondary objectives were evaluation of the safety and tolerability of the combination of hydroxychloroquine and atorvastatin over 24 weeks, and evaluation of the impact of the combination of hydroxychloroquine and atorvastatin on pain and function over 24 weeks. Exploratory objectives are ultrasound assessment of synovitis and marker analysis, including markers of cartilage breakdown, metabolism, and inflammation. To date, 6 patients with medial-compartment knee OA have completed 16 weeks of treatment and been assessed at baseline, during treatment, and in follow-up, through examinations, tests and gadolinium-enhanced MRI.

The Primary Endpoint of this study is determination of the proportion of subjects in whom treatment with the combination of hydroxychloroqine and atorvastatin for 24 weeks significantly reduced synovitis, i.e. reduced the synovitis score (Guermazi et al., Ann Rheum Dis. 2011 70(5):805-11. PMID: 21187293) by 4 or more points, as measured by Gd-MRI. Our overriding hypothesis is that, if the combination of hydroxychloroquine and atorvastatin reduces the low-grade synovitis in OA (as measured by Gd-MRI) in this open-label pilot trial, it will provide chondroprotective effects and reduce the progression of OA in subsequent Phase II and Phase III clinical trials.

The Secondary Endpoints included (1) evaluation of the safety and tolerability of the combination of hydroxychloroquine and atorvasatin in subjects with early-stage OA; (2) determination of the change from baseline in the WOMAC pain subscale, the WOMAC function subscale, the Patient's Global Visual Activity Scale (VAS), the Physician's Global VAS, and the HAQ-DI after 4, 12, and 24 weeks of treatment; (3) analysis of efficacy data by using the OMERACT-OARSI Responder Index (Onel et al, Clin Drug Investig. 2008; 28(1):37-45. PMID: 18081359); and (3) analysis of the use of rescue medications required at 4, 12 and 24 weeks.

Subjects with OA were recruited and informed consent was obtained. During a screening period lasting up to 34 days, subjects provided their medical histories, including arthritis history, underwent physical examination, and completed the WOMAC pain and function subscale questionnaires and patient VAS global assessment questionnaires. ECG, bilateral knee x-rays, and MRI of the index knee was performed and medications being taken were recorded. Samples were obtained for urinalysis, hematological analysis, analysis of blood chemistry, and a urine pregnancy test (for women of childbearing potential). Vital signs and weight are recorded. Subjects are asked to maintain their usual dose of NSAIDs and/or other analgesics during the course of the trial, except for acetaminophen during the 48-hour or 24-hour period preceding efficacy assessments (WOMAC and HAQ questionnaires, and Patient Global Assessment VAS) at each time point.

Subjects who met all of the inclusion criteria and none of the exclusion criteria (listed below) were entered into the study and received the combination of hydroxychloroquine and atorvastatin. Additional follow-up visits were conducted at Weeks 2, 4, 12 and 24 and safety and efficacy assessments performed according to the Schedule of Assessments. Telephone follow-up visits will occur at Weeks 8, 16, and 20. The dosing regimen was hydroxychloroquine sulfate 400 mg/d and atorvastatin calcium 40 mg/d.

Inclusion Criteria:

1. Ambulatory subjects who have had symptomatic OA of the knee for at least 6 months and pain on most days in the last 30 days. Symptoms must include knee joint pain. In subjects with bilateral knee OA, the more symptomatic knee is considered the index knee. 2. Men or women >40 years of age with a body mass index <35. 3. Radiographic evidence of at least one osteophyte in the index knee, as determined by posteroanterior (PA) and lateral standing, flexed x-ray. 4. An OARSI Atlas joint space narrowing grade of 1 or 2 in the index knee. 5. A WOMAC pain score of >8 on the index knee at screening visit 2 and at Day 1/baseline visit. 6. A synovitis score of 9-14 based on gadolinium-enhanced MRI (Gd-MRI) of the index knee and the scoring system (based on summed scores from 11 sites) described in Guermazi et al (Ann Rheum Dis. 2011 70(5):805-11. PMID: 21187293). 7. Ability to comply with the study instructions and give informed consent. 8. Ability to read, write, and understand English.

Exclusion Criteria:

1. Having a requirement for treatment with high-potency opioids for pain relief. 2. Unwilling to abstain from NSAIDs or other analgesic medications except acetaminophen (i.e., COX-2 inhibitors, tramadol) for 48 hours and acetaminophen for 24 hours prior to pain assessments during the study. Subjects taking low-dose aspirin for cardiovascular health may remain on their stable dose throughout the study. 3. Having been on a variable dose of NSAIDs or analgesics for at least 3 months prior to screening visit 1. 4. Using a handicap assistance device (i.e., cane, walker)>50% of the time. 5. Undergoing new physical therapy or participating in a weight-loss or exercise program that has fluctuated during at least 3 months prior to screening visit 1 and will continue to fluctuate during the study. 6. Having a history of arthroscopic or open surgery of the index knee in the past 6 months or planning to have such surgery during the study follow-up. 7. Having had joint replacement surgery of the index knee. 8. Having received corticosteroid, short-acting hyaluronic acid, or other intra-articular injection of the index knee within 3 months of screening visit 1 and/or not willing to abstain from such treatments for the duration of the study. 9. Having a history in the past 5-10 years of reactive arthritis, RA, psoriatic arthritis, ankylosing spondylitis, arthritis associated with inflammatory bowel disease, sarcoidosis, amyloidosis, or fibromyalgia. 10. Having clinical signs and symptoms of active knee infection or radiographic evidence of crystal disease other than chondrocalcinosis (i.e., gout and calcium pyrophosphate crystal disease [CPPD]). 11. Having a history of abnormal laboratory results >2.5× upper limit of normal [ULN] indicative of any medical disease that, in the opinion of the investigator, would preclude participation in the study. 12. Having any of the following abnormal laboratory results during screening: a. ALT and AST >2.5×ULN b. Hemoglobin <9 g/dL c. WBC <3500 cells/mm³. d. Lymphocyte count <1000 cells/mm³ e. Serum creatinine >1.5×ULN. 13. Having a history of malignancy in the past ten years (<10 years), with the exception of resected basal cell carcinoma, squamous cell carcinoma of the skin, or resected cervical atypia or carcinoma in situ. 14. Having significant hip pain, ipsilateral to the index knee, that may interfere with assessments of index knee pain. 15. Having a known or clinically suspected HIV, HCV, or HBV infection. 16. Having participated within 3 months of screening visit 1 or planning to participate concurrently in another investigational drug or vaccine study. 17. Having a history of drug or alcohol dependence or abuse in the past 3 years 18. Being a woman with reproductive capability who is unwilling to use birth control for the duration of the study and/or intends to conceive within 12 months of the start of the study. 19. Having other serious, non-malignant, acute or chronic medical or psychiatric illness that, in the judgment of the investigator, could compromise subject safety, limit the subject's ability to complete the study, and/or compromise the objectives of the study.

All subjects are monitored for adverse events (AEs) during the study. Assessments may include monitoring of any or all of the following parameters: the subject's clinical symptoms; laboratory, pathological, radiological, or surgical findings; physical examination findings; and other appropriate tests and procedures. AEs that cause a subject to discontinue study participation will be followed up until either the event resolves, stabilizes, or returns to baseline (if a baseline assessment is available).

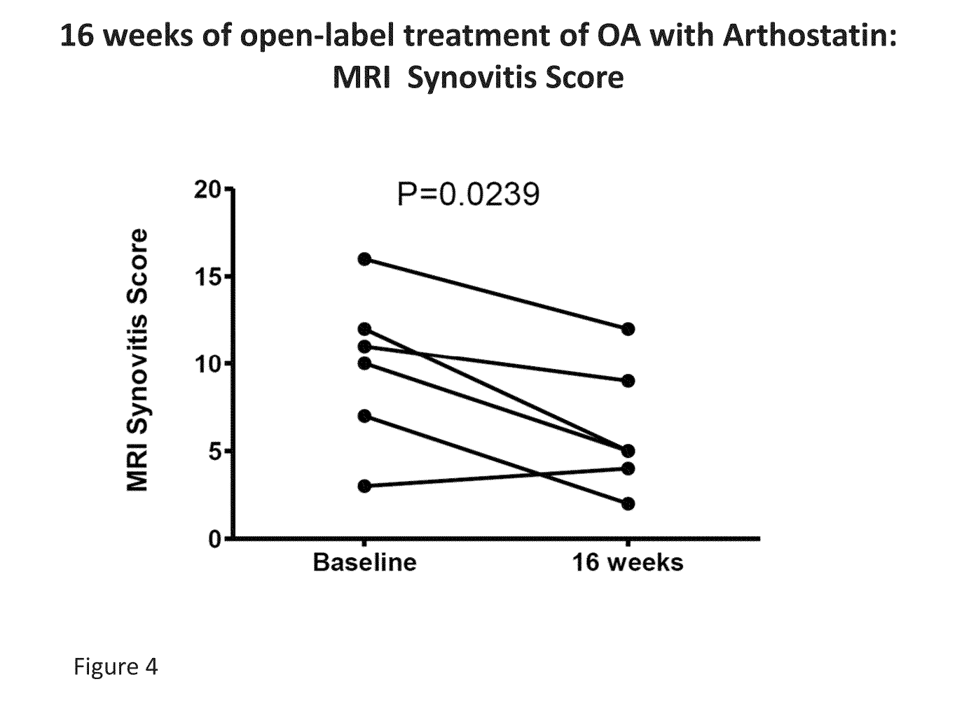

To date, 6 patients with medial-compartment knee OA have met inclusion criteria, been enrolled, and completed 16-weeks of treatment. None of the subjects who started taking the combination of hydroxychloroquine and atorvastatin in our trial have dropped out of our study. There have been no serious AEs in the trial. All 6 subjects have now completed 16 weeks of treatment, including all baseline, in-life and follow-up examinations, tests and gadolinium-enhanced MRI imaging studies. As shown in FIG. 4, the combination of hydroxychloroquine and atorvastatin reduced joint inflammation in humans with medial-compartment knee OA in this 16-week, open-label clinical trial. The MRI Synovitis Score was determined by gadolinium-enhanced MRI scanning of the index knee of each subject at baseline and at the end of the 16-week in-life treatment period, and represents the degree of inflammation in the joint. Subjects were treated with a combination of hydroxychloroquine sulfate 600 mg by mouth each day and atorvastatin calcium 40 mg by mouth each day for 16 weeks. The MRI Synovitis Scores were analyzed by two-way paired t test, an analysis that demonstrated that treatment with the combination of hydroxychloroquine and atorvastatin significantly reduced the amount of synovitis (inflammation) in the index knee joints (P=0.024) (FIG. 4).

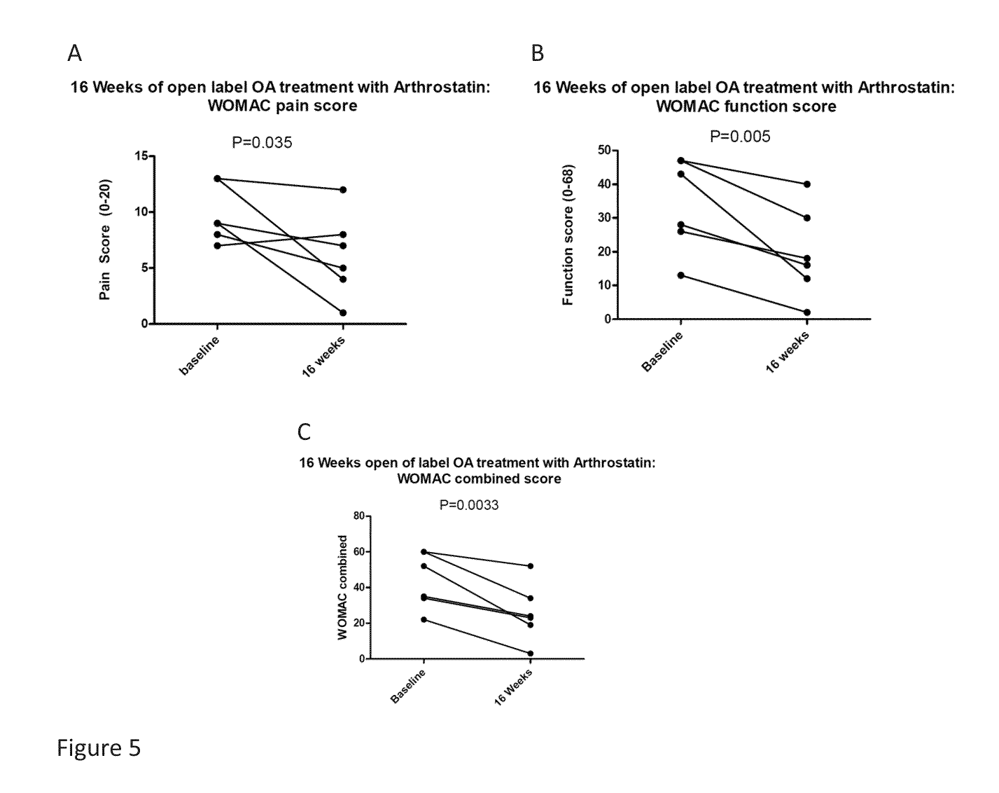

In this trial, we also measured Western Ontario and McMaster Universities Arthritis Index (WOMAC) Pain, Functional and Combined Scores (see McConnell et al., The Western Ontario and McMaster Universities Osteoarthritis Index (WOMAC): a review of its utility and measurement properties. Arthritis Rheum2001; 45: 453-61. PMID:11642645). The WOMAC Pain, Function and Combined scores were analyzed by one-tailed T tests, which demonstrated that treatment with a combination of hydroxychloroquine and atorvastatin statistically reduced the WOMAC Pain Score at 16 weeks (P=0.035), WOMAC Function Score (P=0.005), and the WOMAC Combined Score (P=0.003) (FIG. 5).

Thus, our 16-week, open-label pilot trial of the combination of the combination of hydroxychloroquine and atorvastatin in humans with medial-compartment knee OA demonstrates that this combination reduced synovitis (inflammation) in the affected knee (P=0.024; FIG. 4), and resulted in improvements in the WOMAC Pain, Function, and Combined Scores (FIG. 5). Together, these data suggest that the combination of hydroxychloroquine and atorvastatin provided meaningful clinical benefit to humans with knee OA, reduced inflammation in their joints, and thus may attenuate the progression of OA in humans.

In larger and longer phase II and phase III human trials, drugs that reduce inflammation are anticipated to provide disease-slowing effects including chondroprotection (e.g. reduction in the rate of cartilage breakdown). Specifically, the combination of hydroxychloroquine and atorvastatin by reducing synovitis is anticipated to result in a slowing of OA disease progression. This slowing of OA disease progression in subsequent phase II and phase III trials will be demonstrated by preservation of joint space (e.g. slowing of the narrowing of the joint space in the medial compartment of the index knee, as determined by weight-bearing plain film X-rays of the index knee), or preservation of cartilage volume or integrity as demonstrated by MRI or other imaging of the index knee.

New methods are being developed for measuring cartilage volume and integrity, and these new methods will be used in subsequent phase II and phase III studies to demonstrate that treatment with the combination of hydroxychloroquine and atorvastatin protects against cartilage loss in human OA. An example of the methods for analyzing joint-space narrowing by plain X-ray in medial-compartment knee OA are described in Brandt et al. (Arthritis and Rheumatism, 52(7):2015-2025, PMID: 15986343), and the slowing of joint-space narrowing is considered to demonstrate disease-slowing activity in OA. A second and more sensitive method to demonstrate chondroprotection is the demonstration of preservation of cartilage volume on MRI scan, and an example of methods of using MRI to demonstrate cartilage volume preservation are described in Raynauld et al. (Ann Rheum Dis. 2009, 68(6):938-47).

### Example 3

### Use of Combination Therapy with Hydroxychloroquine and Atorvastatin to Inhibit Development of and to Reduce the Severity of Osteoarthritis (OA)

Humans are screened for evidence of early-stage OA or pre-clinical OA or being at increased risk of developing OA. Many factors can increase an individual's risk of developing OA, for example, joint injury, joint surgery, degenerative meniscal tears, degeneration of articular cartilage, anterior cruciate ligament tears, defects in collagen or other matrix proteins, genetic predisposition, etc. Humans with pre-clinical or early-stage OA are asymptomatic, or have mild or intermittent joint pain, and can be treated with the combination of hydroxychloroquine and atorvastatin to prevent the development of the symptoms and signs of clinical OA, as well as to prevent the progression of pre-clinical or early-stage OA. Further, humans at risk for OA, with pre-clinical OA or with early-stage OA can be tested for the presence of inflammation in the affected joint to identify individuals who are likely to respond to treatment with the combination of hydroxychloroquine and atorvastatin. Testing for joint inflammation can be performed using imaging markers, such as MRI with or without gadolinium contrast, or ultrasound, and by determining the presence of one or more of the following features indicative of inflammation: synovial enhancement or proliferation, joint effusion, and bone marrow edema. Molecular markers of inflammation can also be tested for, including one or more of CRP, ESR, and inflammatory cytokines. Finally, clinical history and examination can be used to assess inflammation—including the presence of an effusion on physical exam or morning stiffness in the clinical history. Individuals at-risk for, with pre-clinical or with early-stage stage OA who exhibit elevations in inflammatory markers as compared to healthy control individuals can be treatment with the combination of hydroxychloroquine and atorvastatin to prevent the development of the symptoms and signs of clinical OA, as well as to prevent the progression of pre-clinical or early-stage OA. Alternatively, they can be treated with a combination of desethylhydroxychloroquine and atorvastatin to prevent the development of the symptoms and signs of clinical OA, as well as to prevent the progression of pre-clinical or early-stage OA.

Hydroxychloroquine sulfate is generally given as a 400 mg/day (which is 310 mg/day hydroxychloroquine base, which for an individual with a body weight of 70 kg is 4.4 mg/kg/day of hydroxychloroquine base), and the dose of HCQ sulfate can be between 100-600 mg/day (which is 77.5-465 mg HCQ base; 1.1-6.64 mg/kg/day of HCQ base). Atorvastatin calcium is generally dosed at an atorvastatin base dose of 10, 20, 30 or 40 mg/day (0.14-0.57 mg/kg/day of atorvastatin base), but the atorvastatin base dose can be between 5 and 80 mg/day of atorvastatin base (0.07-1.1 mg/kg/day)).

The HCQ and atorvastatin components can be delivered in individual tablets or capsules, or in a combined tablet or capsule that includes both drugs. The HCQ and atorvastatin components can be delivered in 1 tablet or capsule one time per day, 2 tablets or capsules one time per day, 3 tablets or capsules one time per day, or 4 tablets or capsules one time per day, or more than 4 tablets or capsules one time per day. The HCQ and atorvastatin components can be delivered in 1 tablet or capsule two times per day, 2 tablets or capsules two time per day, or more than 2 tablets or capsules 2 or more times per day.

Examples of humans at risk of developing OA, and their eligibility for treatment with the combination of hydroxychloroquine and atorvastatin are:

(1) A 59-year-old man with intermittent knee pain is diagnosed with early-stage OA of the right knee (Kellgren-Lawrence, K-L, grade I). His ability to run is limited due to knee pain experienced on running. Range of motion in his R knee is not compromised, and there is no deformity of angulation of adduction moment on ambulation. The WOMAC OA index is used for assessing pain and his WOMAC pain score is 6. The patient undergoes MRI with gadolinium of the R knee, which reveals enhancement consistent with synovitis as assessed with a semiquantitative scoring system. The patient is treated with the combination therapy consisting of hydroxychloroquine sulfate 400 mg and atorvastatin calcium 40 mg, each taken once daily as a combination capsule. Another MRI is performed six months later.

(2) A 44-year-old male amateur rugby player develops pain and clicking in his left knee, symptoms that appear when he runs. He is evaluated by X-ray, which demonstrates K-L grade 0 (normal X-ray), and by knee MRI which reveals a posterior meniscal tear and cartilage edema; his WOMAC pain score is 2; he is scheduled for arthroscopic debridement. Blood tests reveal a CRP level of 3.1. Beginning one month before surgical debridement, the patient is treated with HCQ sulfate 100 mg daily for 1 week, then 600 mg daily for 8 weeks, then 400 mg daily thereafter, taken in combination with atorvastatin calcium 30 mg daily.

(3) A 54-year-old man presents with mild, intermittent locking of his left knee. X-ray reveals K-L grade 1 OA, and ultrasound reveals a degenerative meniscal tear, as well as moderate synovial enhancement consistent with synovitis. The patient is offered arthroscopic meniscal debridement but declines surgical intervention. He is prescribed hydroxychloroquine sulfate 400 mg and atorvastatin calcium 40 mg daily.

(4) A 28-year-old man develops a fracture of his right ankle (tibial plafond) with appropriate reduction and casting. X-rays analysis does not show any features characteristic of OA. Given the 30% risk of his developing radiographic OA 2-4 years after sustaining the fracture, and a 74% risk 11 years after sustaining the fracture, the patient is monitored for evidence of joint inflammation by ultrasound and MRI, and/or by testing for the presence of molecular markers. Ultrasound detects a synovial effusion and synovitis, and based on these findings the patient is started on a combination of HCQ sulfate 400 mg/day plus atorvastatin calcium 20 mg/day and does not develop evidence of radiographic OA.

### Example 4

### Treatment with the Combination of Hydroxychloroquine and Atorvastatin Prevented Development of and Reduced the Severity of Murine Rheumatoid Arthritis (RA)

Mouse Model of RA.

8-week-old male DBA/1 mice (Jackson Laboratory) were used for generating the collagen-induced arthritis (CIA) mouse model of RA. Experiments were performed under protocols approved by the Committee of Animal Research at Stanford University and in accordance with NIH guidelines. DBA/1 mice were intradermally immunized with 100 μg/mouse of bovine collagen type II (Chondrex) emulsified in complete Freund's adjuvant (CFA) containing 250 μg/mouse of heat-killed Mycobacterium tuberculosis H37Ra (BD). 21 days after immunization, mice were subcutaneously injected at the base of the tail with 100 μg/mouse of bovine CII emulsified in incomplete Freund's adjuvant (IFA). Before approximately day 28, the mice have no symptoms of RA but, owing to the collagen immunization, are in a state of pre-clinical or early-stage RA characterized by elevations in inflammatory markers in the blood. Further, by day 14 the immunized mice have mounted an autoantibody response against type II collagen, the autoantibody response has undergone epitope spreading, and the mice consequently have inflammation associated with pre-clinical RA (Arthritis Res Ther. 2008; 10(5):R119. PMID: 18826638; Finnegan et al, Autoimmunity. 2012 45(5):353-63. PMID: 22432771). Mice start to manifest clinical RA at approximately day 28, and inflammatory arthritis in the mice was evaluated by visually scoring limb inflammation, measuring paw thickness, and weighing spleens. The visual scoring system was as follows: grade 0, no swelling or erythema; grade 1, mild swelling and erythema or digit inflammation; grade 2, moderate swelling and erythema confined to the region distal to the mid-paw; grade 3, more pronounced swelling and erythema extending to the ankle; grade 4, severe swelling, erythema, and joint rigidity of the ankle, foot, and digits. Each limb was graded with a score of 0-4, with a maximum possible score of 16 for each individual mouse. Paw thickness was determined by measuring the thickness of both hind paws with O— to 10-mm calipers and calculating the mean of the two measurements.

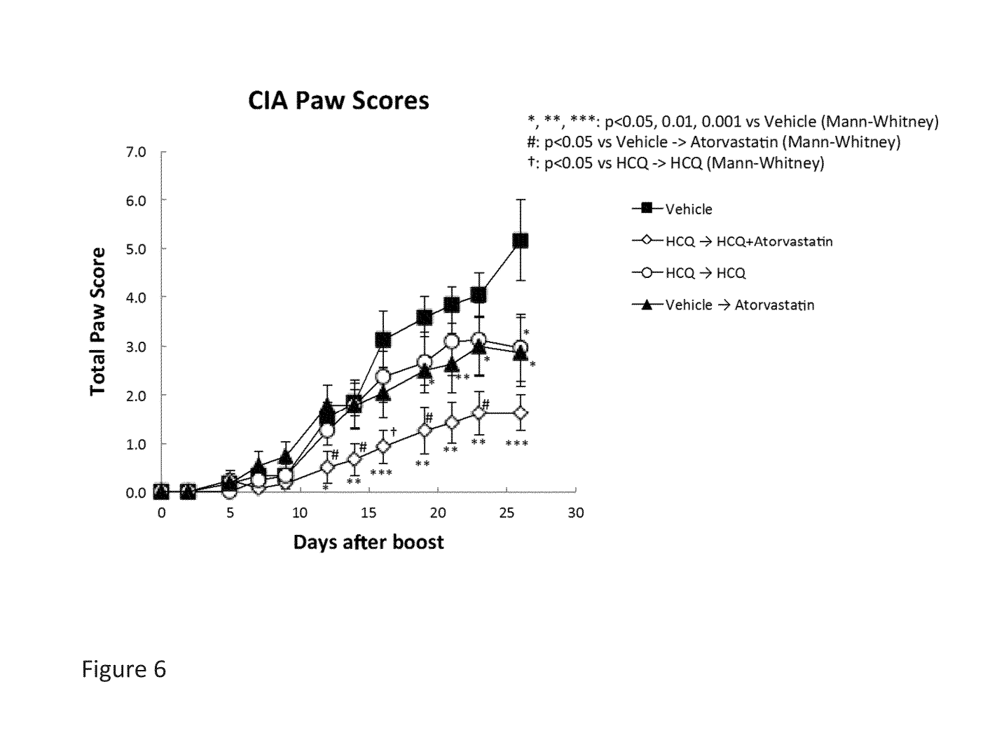

On the day of the first immunization, treatment was initiated with HCQ 50 mg/kg/day by oral gavage for 2 weeks, then increased to 100 mg/kg/day by oral gavage for one week. Starting at the time of boosting (i.e., immunization with IFA; day 21), mice were treated with HCQ 50 mg/kg/day and atorvastatin calcium 10 mg/kg/day by oral gavage. Mice in the control group were treated with vehicle alone. Severity of arthritis in the mice was scored according to the visual scoring system, revealing that mice developed arthritis approximately one week after the boosting (i.e., 28 days after the initial immunization). Arthritis was significantly less severe in mice treated with HCQ plus atorvastatin as compared to the mice in the other treatment or vehicle control groups (P<0.05 by t test) (FIG. 6). Synovitis, pannus formation, and bone erosion were also lower in mice treated with HCQ plus atorvastatin than in mice treated with HCQ alone, atorvastatin alone, or vehicle (P<0.05 by t test).

Thus, we demonstrated that a combination of HCQ plus atorvastatin reduced the severity of inflammatory arthritis in a mouse model of RA.

### Example 5

### Use of Combination Therapy with Hydroxychloroquine and Atorvastatin to Prevent Development of Rheumatoid Arthritis (RA)

Humans are screened for evidence of early-stage RA or increased risk of developing RA (e.g. having pre-clinical RA). Findings that suggest an individual has early-stage RA include one or more of the following: the presence of one or more swollen joints, the presence of anti-CCP or RF antibodies, the presence of synovial enhancement as determined by MRI or ultrasound, and molecular markers demonstrated to provide predict the later development of RA (as described in Sokolove et al, PLoS One. 2012; 7(5):e35296, PMID: 22662108). Factors associated with an increased risk of developing RA include one or more of the following: a family history of RA (particularly in a first-degree relative), increased levels of anti-CCP and/or RF antibodies, a genetic profile associated with susceptibility to RA, and/or synovitis in one or more joints.

Further, humans at risk for RA, with pre-RA or with early-stage RA can be tested for the presence of inflammation in the involved joint to identify individuals who are likely to respond to treatment with the combination of hydroxychloroquine and atorvastatin. Testing for joint inflammation can be performed by MRI, with or without gadolinium contrast, or ultrasound, to determine whether one or more of the following features are present: synovial enhancement or proliferation, joint effusion, and bone marrow edema. Molecular markers of inflammation can also be tested for, including one or more of CRP, ESR, and inflammatory cytokines. Finally, clinical history and exam can be used to assess inflammation—including the presence of synovitis on physical examination, an effusion on physical exam, or morning stiffness lasting >1 hour on history.

Individuals at increased risk of developing RA or with features of early-stage RA, particularly those who have signs of inflammation, as evidenced by imaging, molecular or clinical markers, can be treated with the combination of hydroxychloroquine and atorvastatin to prevent the onset or progression of clinical RA. Hydroxychloroquine sulfate is generally given as a 400 mg/day (which is 310 mg/day hydroxychloroquine base, which for an individual with a body weight of 70 kg is 4.4 mg/kg/day of hydroxychloroquine base), and the dose of HCQ sulfate can be between 100-600 mg/day (which is 77.5-465 mg HCQ base; 1.1-6.64 mg/kg/day of HCQ base). Atrovastatin calcium is generally dosed at an atorvastatin base dose of 10, 20, 30 or 40 mg/day (0.14-0.57 mg/kg/day of atorvastatin base), but this dose can be between 5 and 80 mg/day of atorvastatin base (0.07-1.1 mg/kg/day)).

The HCQ and atorvastatin components can be delivered in individual tablets or capsules, or in a combined tablet or capsule that includes both drugs. The HCQ and atorvastatin components can be delivered 1 time per day. The HCQ and atorvastatin components can be delivered by 2 tablets or capsules taken 1 time per day. The HCQ and atorvastatin components can be delivered 2 times per day.

Examples of humans at risk of developing RA, and their treatment with the combination of hydroxychloroquine and atorvastatin are as follows:

(1) A 49-year-old woman with 4 months of bilateral morning stiffness in her hands and wrists is found to test positive for anti-CCP autoantibodies, and to have an ESR of 49 and a CRP value of 4.1. On examination, she has bilateral tenderness and swelling in her wrists as well as in several metacarpal-phalangeal and proximal interphalangeal joints. Her disease activity score, which includes evaluation of 28 joints (DAS23 score), is 4.1. She is diagnosed with early-stage RA and prescribed HCQ sulfate 400 mg and atorvastatin calcium 40 mg daily. On follow-up evaluation 10 weeks later, she feels significantly improved, her DAS28 score is now 2.3, and she has not developed bone erosions.

(2) A 32-year-old woman has a sister with RA and is found to test positive for anti-CCP autoantibodies. She does not have features consistent with the diagnostic criteria for RA. Her rheumatologist orders marker analysis that reveals elevations in levels of multiple autoantibodies and cytokines, as described in (Sokolove et al, PLoS One. 2012; 7(5):e35296, PMID: 22662108), placing her at increased risk of developing RA within the following 2 years. She is prescribed HCQ sulfate 400 mg and atorvastatin calcium 30 mg daily. On annual follow-up evaluations, she does not have symptoms or signs of RA.

(3) A 50-year-old man develops swelling in two metacarpalphalangeal (MCP) joints and is found to test positive for anti-CCP autoantibodies. He does not have features consistent with the diagnostic criteria for RA. Ultrasound performed by his rheumatologist demonstrates low-grade, bilateral synovitis in his wrists and in multiple MCP joints. His rheumatologist therefore believes him to be at increased risk of developing RA, and the patient is prescribed HCQ sulfate 400 mg and atorvastatin calcium 40 mg daily. On annual follow-up evaluations, the swelling in his MCPs and wrists is reduced, as determined by both ultrasound and physical examination, and he continues to do well without developing overt RA.

(4) A 60-year-old woman develops bilateral swelling in her wrists, MCP joints, and PIP joints that has persisted for 5 months, is found to test positive for RF and anti-CCP autoantibodies, and to have an ESR of 55 and a CRP value of 4.1. MRI of her hands and wrists demonstrates synovial thickening and proliferation indicative of synovitis. She is diagnosed with early-stage RA, and her rheumatologist determines her DAS score to be 4.5. Her rheumatologist prescribes HCQ sulfate 400 mg and atorvastatin calcium 40 mg daily. On annual follow-up evaluations, her joint swelling and arthritic symptoms have improved, and her DAS score has decreased to 3.2.

### Example 6

### Treatment with the Combination of Hydroxychloroquine and Atorvastatin Prevented the Development of and Reduced the Severity of the EAE Mouse Model of Multiple Sclerosis (MS)

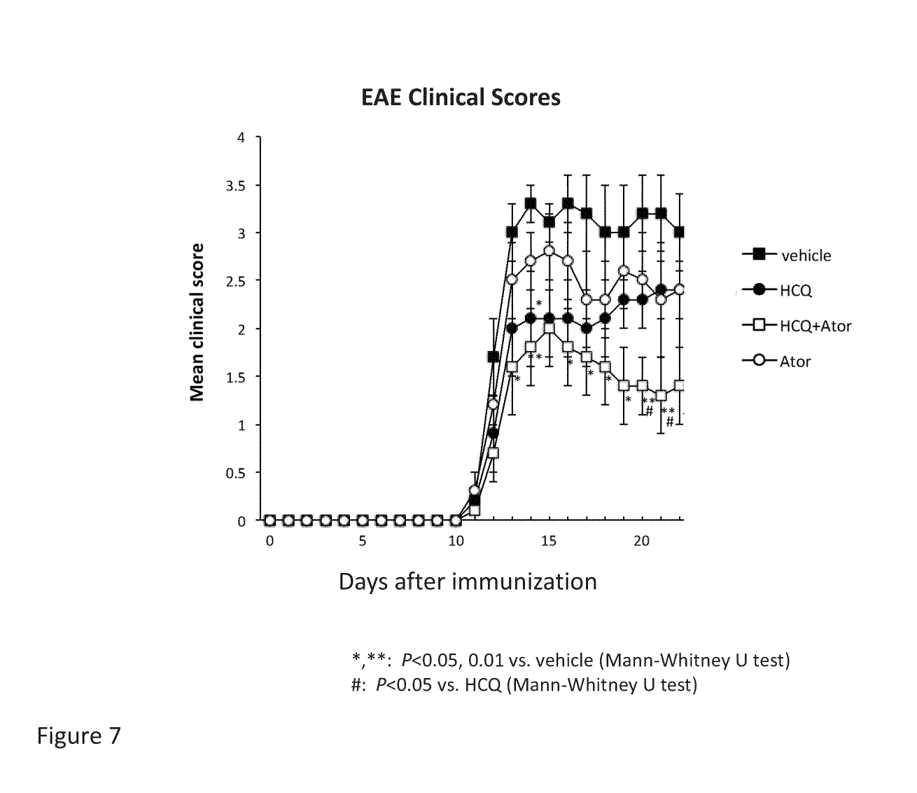

Experimental autoimmune encephalomyelitis (EAE), a mouse model of MS, was induced in SJL mice (n=10 per group) by immunization with proteolipid protein peptide 139-151 (PLP 139-151) in CFA. Starting at the time of immunization, mice were treated with a loading dose of HCQ sulfate 100 mg/kg/day in combination with atorvastatin calcium 1 mg/kg/day, and 8 later the dosing regimen was changed to HCQ 50 mg/kg/day in combination with atorvastatin 1 mg/kg/day. This loading-dose regimen is used to rapidly achieve therapeutic levels of HCQ in the tissues at the start of therapy, and then the dose is reduced to the maintenance dose for long-term therapy. For the first approximately 10 days after the initial immunization, the mice exhibit no symptoms of MS, but they are inflamed, develop autoantibodies, and are considered to have pre-clinical or early-stage MS. Starting eight days after immunization, mice were scored daily for the severity of EAE. Mann Whitney U test comparisons between the groups demonstrated that treatment with HCQ plus atorvastatin results in significantly less severe disease as compared to treatment with HCQ alone or atorvastatin alone (FIG. 7).

Thus, we demonstrated that a combination of HCQ plus atorvastatin reduced the severity of the EAE mouse model of MS, and that the reduction in inflammation correlated positively with the reduction in disease severity.

### Example 7

### The Combination of Hydroxychloroquine and Atorvastatin Reduced Insulin Resistance and Hyperglycemia in a Mouse Model of Type II Diabetes and Metabolic Syndrome

To evaluate the effect of combination therapy with HCQ plus atorvastatin on animal models of hyperlipidemia, Type II diabetes, and NAFLD, C57BL/6 mice (5 per group) were fed a high-fat diet (Taconic) for 6 weeks. During the final 4 weeks of the high-fat diet, mice were treated with the combination of HCQ sulfate (100 mg/kg/day) plus atorvastatin calcium (40 mg/kg/day), or vehicle control, and non-fasting serum samples were then collected for analysis. The combination of hydroxychloroquine and atorvastatin prevented development of and reduced the levels of inflammation-related metabolic and tissue injury biomarkers in this mouse model of diet-induced obesity (DIO). For assessing the effect of combination therapy with hydroxychloroquine plus atorvastatin on mouse models of hyperlipidemia, type II diabetes, and non-alcoholic fatty liver disease (NAFLD), C57BL/6 mice (n=5 per group) were fed a high-fat “western-style” diet (Taconic) for 10 weeks. The mice exhibited normal behavior and no overt symptoms throughout this time, but developed a pre- or early-disease state as evidence by elevations in blood glucose, cholesterol, triglycerides. During the final 4 weeks of the high-fat diet, these asymptomatic pre-disease mice were treated with the combination of hydroxychloroquine sulfate (HCQ; 100 mg/kg/day) plus atorvastatin calcium (Atorv; 40 mg/kg/day), or with vehicle. After 6 weeks of treatment, non-fasting sera were analyzed.

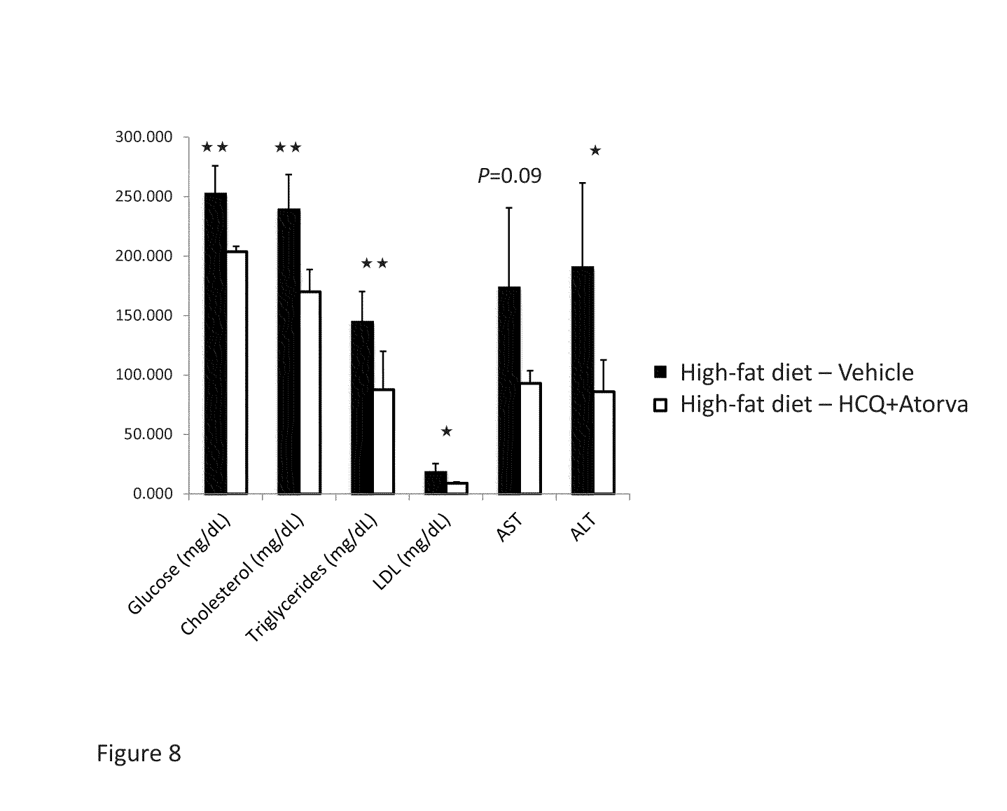

As demonstrated in FIG. 8, the levels of total cholesterol (P<0.01), triglycerides (P<0.01), and LDL cholesterol, i.e., inflammation-related metabolic markers, were significantly lower in mice treated with the combination of HCQ plus atorvastatin than in mice treated with vehicle. In addition, levels of glucose, a biomarker of early insulin resistance and early-stage type II diabetes, were significantly lower in mice treated with the combination of HCQ and atorvastatin than in mice treated with vehicle control (P<0.01, by two-tailed t test). Further, serum levels of ALT (also known as serum glutamic pyruvate transaminase [SGPT]), a biomarker of NASH, were significantly lower in mice treated with the combination of HCQ plus atorvastatin (P<0.05); there was a similar trend with levels of serum aspartate transaminase (AST; also known as serum glutamic oxaloacetic transaminase [SGOT]) (P=0.09). These data demonstrate that treatment with the combination of HCQ plus atorvastatin prevented and treated early insulin resistance, which represents a pre-clinical or early-stage of type II diabetes and/or metabolic syndrome. These data also demonstrate that treatment with the combination of HCQ plus atorvastatin can treat hypercholesterolemia and thus prevent the development of atherosclerosis. Further, they demonstrate that treatment with a combination of HCQ plus atorvastatin can treat NAFLD, thereby preventing the development of NASH. Together, these data suggest that treatment with the combination of HCQ plus atorvastatin prevents the development of and treats the early stages of metabolic syndrome, and prevents the development of metabolic abnormalities and liver injury.

### Example 8

### The Combination of Hydroxychloroquine and Atorvastatin Reduced Hepatic Inflammation in a Murine Model of Non-Alcoholic Fatty Liver Disease (NASH)

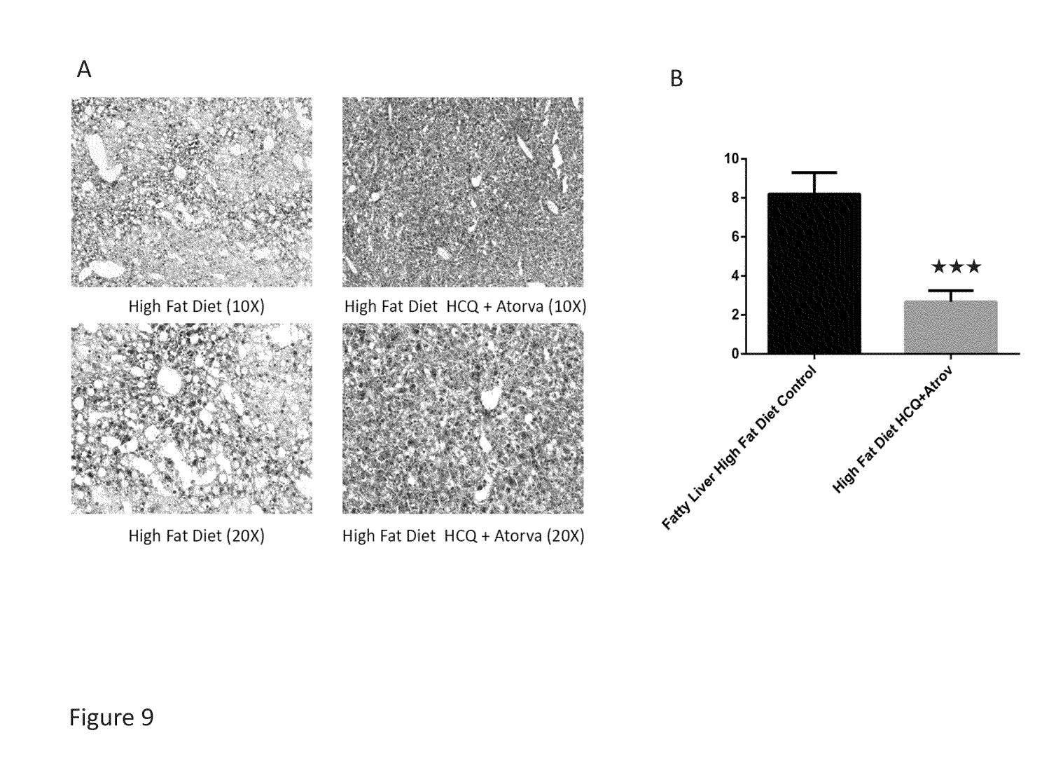

The combination of hydroxychloroquine and atorvastatin prevented the development of fatty liver and liver injury in a mouse model of diet-induced obesity (DIO). From the experiment in FIG. 8, following 6 weeks of high-fat diet and dosing with the combination of HCQ and Atorva mice were sacrificed, and their livers harvested. Livers were formalin-fixed, paraffin-embedded, sectioned and stained with hematoxylin and eosin (H&E) (FIG. 9A). Liver histology was examined under a light microscope and then graded according to the magnitude of steatosis, inflammation, and ballooning degeneration of hepatocytesas based on an established scoring system (Brunt et al, American Journal of Gastroenterology, 94(9):2467-2474, 1999) (FIG. 9B). Briefly, the degree of steatosis was graded 0-4 based on the average percent of fat accumulated hepatocytes per field 200× under H&E staining (grading: 0=<5%, 1=5-25%, 2=26-50%, 3=51-75%, 4=>75%). Inflammation was evaluated by the number of inflammatory cells counted in 10 random fields at 200× magnification. The mean of these numbers was calculated and regarded as inflammatory cells/mm2. Hepatocellular ballooning degeneration was evaluated as either negative (absent=0), positive (present=1), or dominant (present and dominant=2). The scoring of liver histology demonstrated that the treatment with the combination of HCQ and atorvastatin statistically reduced liver steatosis and injury (P<0.001, by t test) (FIG. 9B). These data demonstrate that treatment with the combination of HCQ plus atorvastatin can treat NAFLD, which is expected to reduce progression NASH.

### Example 9

### Both Established and Early Osteoarthritis are Associated with Expression of Inflammatory Mediators

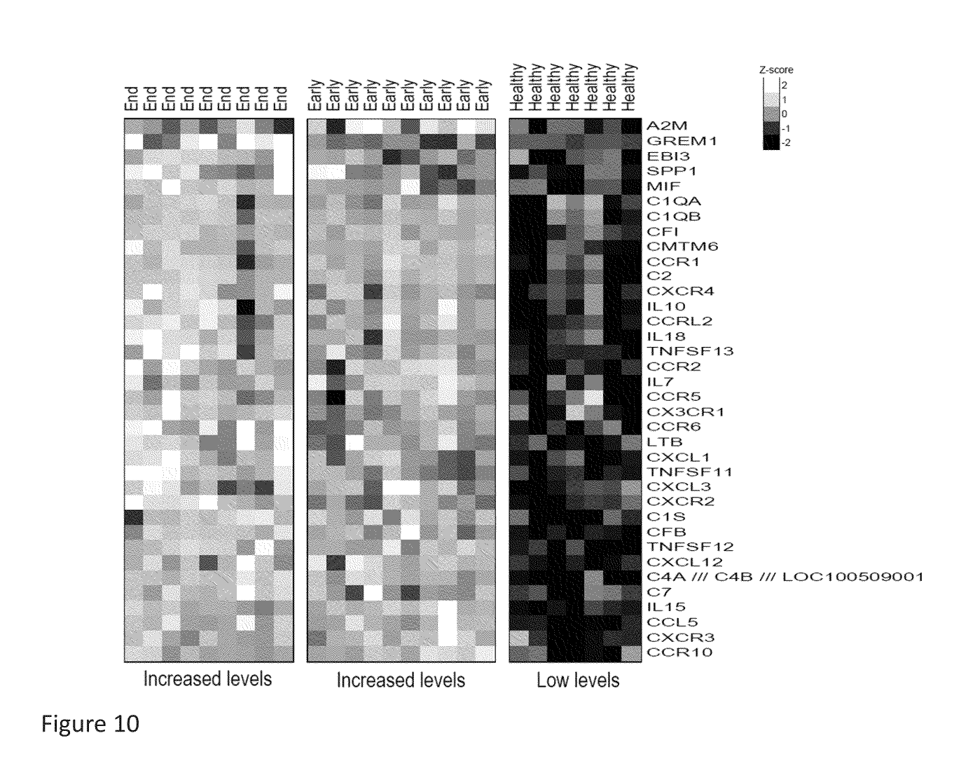

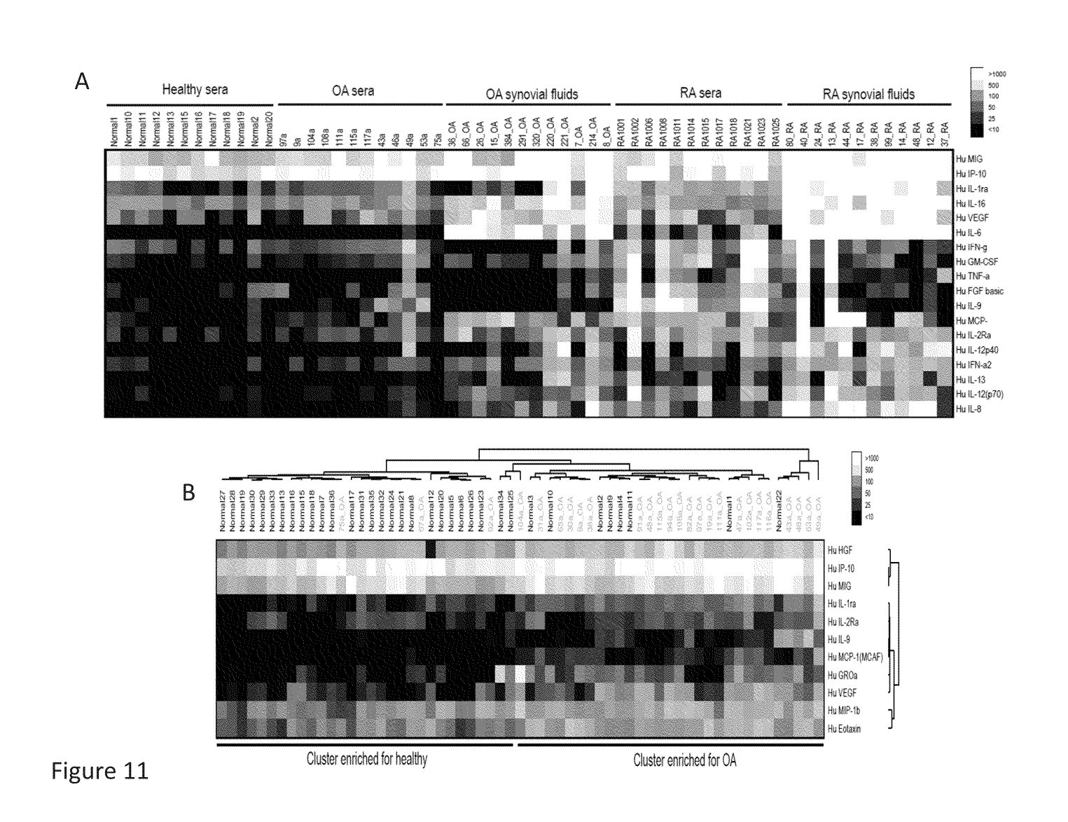

We found that expression of inflammatory mediators is abnormally high both in established OA and in early-stage OA (FIGS. 10 and 11). FIG. 11 demonstrates the identification of inflammatory mediators in synovial fluids derived from humans with established OA. FIG. 10 demonstrates increased expression of genes encoding cytokines, chemokines, complement components, and other inflammatory mediators in synovial tissue derived from humans with early-stage or end-stage OA.

Subjects and Methods.

Serum and synovial fluid samples were obtained from individuals with OA, individuals with rheumatoid arthritis (RA), and healthy individuals under protocols approved by the Stanford University Institutional Review Board and with the patients' informed consent. Synovial fluid aspiration was performed by a board-certified rheumatologist by fine-needle arthrotomy, and the synovial fluid samples obtained were free from obvious contamination with blood or debris. OA serum and synovial fluid samples were obtained from patients diagnosed with knee OA (of Kellgren-Lawrence score 2-4 (Kellgren, J. H., et al., Ann Rheum Dis., 16: 494-502 (1957)) according to the 1985 criteria of the American Rheumatism Association (Altman, R., et al., Arthritis Rheum., 29: 1039-1049 (1986)). For mass spectrometric analysis, OA synovial fluid samples were from five Caucasian men aged 50-75 years meeting the American Rheumatism Association's 1985 criteria for the diagnosis of OA. All RA patients met the 1987 Arthritis College of Rheumatology criteria for RA (Arnett, F. C., et al., Arthritis Rheum., 31: 315-324 (1998)) and had RA of <6 months' duration.

Gene Expression Analysis.

Publicly available gene-expression profiles of synovial membrane derived from OA patients and healthy controls (accession # GSE12021) were downloaded from the NCBI's Gene Expression Omnibus (GEO). The results for expression of genes encoding inflammatory proteins including cytokines, chemokines, complement components, and other mediators were extracted, subjected to hierarchical clustering, and the genes increased in expression in early or end-stage OA are displayed in a heatmap. The relative change in gene expression relative to healthy controls is indicated.

### Example 10

### Both Osteoarthritis and Rheumatoid Arthritis are Associated with Elevations In Blood and Synovial Fluid Cytokines

To further investigate our finding that expression of inflammatory genes is upregulated in early-stage and end-stage OA synovium (FIG. 10), we used a multiplex immunoassay to measure levels of inflammatory cytokines and chemokines in synovial fluid samples derived from 12 patients with knee OA and 14 patients with RA (FIG. 11). We also measured levels of these molecules in serum samples derived from 24 patients with knee OA and 23 patients with RA, as well as in ‘normal’ serum samples derived from 35 healthy individuals. The samples from patients with RA, a classic inflammatory arthritis, were used as a comparator. FIG. 11 shows a heatmap of the relative levels of cytokines in the five groups of samples. Compared to cytokine levels in normal sera, cytokine levels in OA sera were slightly higher and those in RA sera were much higher. Among both OA and RA samples, cytokine levels were much higher in synovial fluids than in sera from patients with the same disease, suggesting that the cytokines are produced locally, in the joints, in both diseases. Significance Analysis of Microarrays (SAM) (Tibshirani, R., et al., Proc Natl Aced Sci USA, 99: 6567-6572 (2002)) analysis revealed that levels of several inflammatory cytokines (e.g. IL-1α and IL-6) and chemokines (e.g. IP-10 (also known as CXCL10), MCP-1, IL-8, and MIP-1α) were significantly higher in OA sera than in normal sera (FDR <10%). The abnormally high levels of cytokines in OA sera may reflect overproduction of these cytokines in the joint. Indeed, levels of high-sensitivity C-reactive protein (hs-CRP) in the serum of OA patients correlates with the degree of inflammatory infiltrate in the patients' joints (Pearle, A. D., et al., Osteoarthritis Cartilage, 15: 516-523 (2007)). As expected, cytokine levels were significantly higher in RA sera than in OA sera (FDR <10%). Nevertheless, our results suggest that OA is associated with low-grade inflammation.

Multiplex Cytokine Analysis.

Multiplex analysis of cytokines and chemokines in human serum and synovial fluid samples was performed using the 27-plex and the 21-plex Bio-Plex Pro Human Cytokine Assay (BioRad) run on the Luminex 200 platform, as recommended by the manufacturers. Data processing was performed using Bio-Plex Manager 5.0, and analyte concentrations (in pg/ml) were interpolated from standard curves. Statistical differences in cytokine levels were calculated by ‘significance analysis of microarrays’ (SAM; Tibshirani, R., et al., Proc Natl Aced Sci USA, 99: 6567-6572 (2002)), and the SAM-generated results with a false discovery rate (FDR) of less than 10% were selected. For identification of relationships and optimal display of the results, the analyte concentrations were analyzed as follows: all values less than 1 were designated as 1, and the mean concentration of each analyte in the ‘normal serum’ samples was calculated; the analyte value in the sample was then divided by the mean analyte value in normal serum, and finally a log-base-2 transformation was applied. Results were subjected to unsupervised hierarchical clustering using Cluster® 3.0, which arranges the SAM-generated results according to similarities in cytokine levels, and the clustering results were finally displayed using Java Treeview® (Version 1.1.3).

### Example 11

### The Combination of Hydroxychloroquine and Atorvastatin Act Synergistically to Reduce Inflammatory Cytokine Production in Response to Multiple Stimuli

The combination of hydroxychloroquine (HCQ) and atorvastatin (Ator) reduced the production of pro-inflammatory cytokines in several cell types from multiple species in response to multiple stimuli.

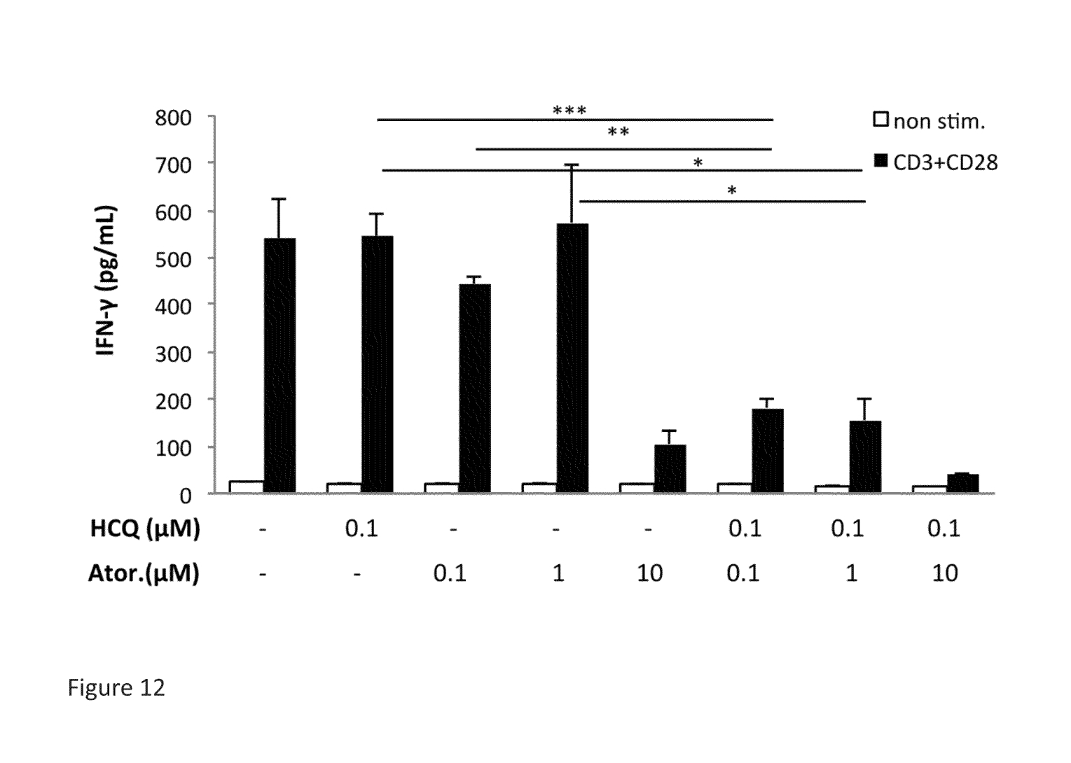

First, the combination of HCQ and atorvastatin synergistically reduced the production of the inflammatory mediator interferon gamma (IFN-gamma) by splenocytes derived from mice with CIA and stimulated in vitro with anti-CD3 and anti-CD28 antibodies (FIG. 12).

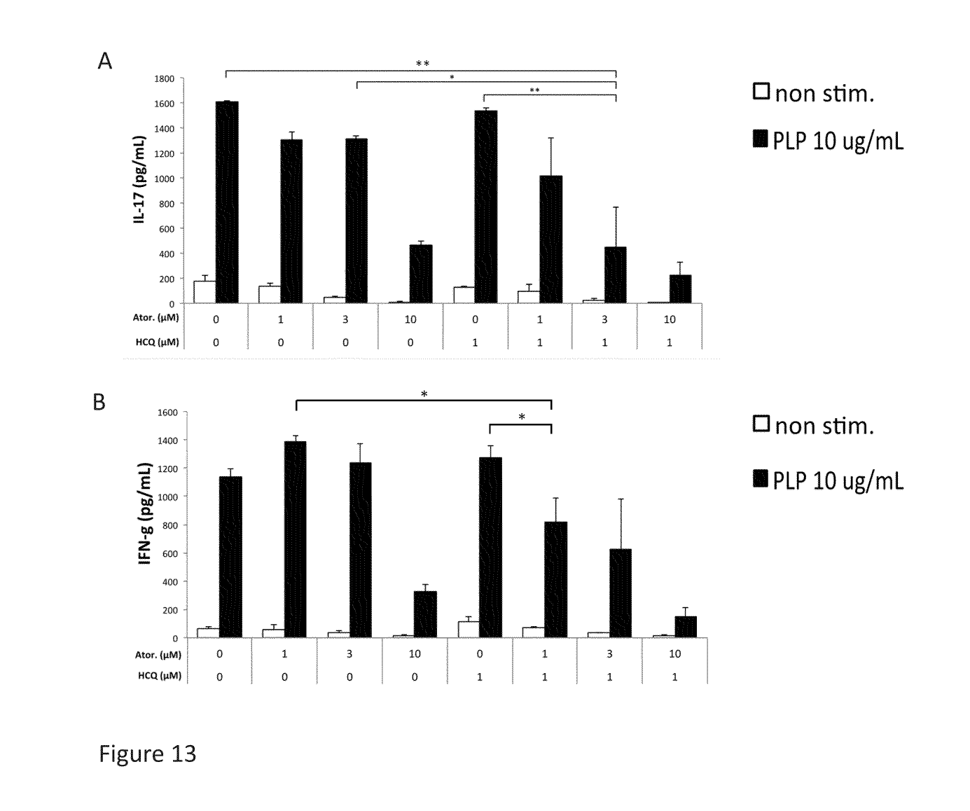

Second, the combination of HCQ and atorvastatin synergistically reduced the production of the pro-inflammatory mediators IFN-γ and IL-17 by splenocytes isolated from mice with EAE and stimulated in vitro with PLP (FIG. 13).

Isolation of Mouse Splenocytes.

Mouse spleens were obtained from naïve mice and from mice with CIA or EAE. Cells were isolated from the spleens by maceration and flushing of cellular material through a 70-micron cell strainer. Cells were washed once with RPMI media without FCS and then resuspended in RPMI containing 10% FCS.

Stimulation Assays.

Human monocytes plated at 5.0×10⁴ cells/well in 96-well culture plates were pretreated with HCQ and atorvastatin for 60 min at 37° C., 5% CO₂ and then stimulated with LPS (Sigma) for 15 h at 37° C., 5% CO₂. Mouse splenocytes plated at 1.0×10⁵ cells/well in 96-well culture plates were pretreated with HCQ, atorvastatin, or both for 60 min at 37° C., 5% CO₂, and then stimulated with Dynabeads® CD3/CD28 T-cell expander (Cat; 114.52D, Invitrogen) at 5.0×10⁴ beads/well, or in the case of EAE splenocytes with PLP (10 ug/ml), for 48 h at 37° C., 5% CO₂. Output from cellular assays was IFN-γ and IL-17 for T-cell stimulation assays using dynabeads or PLP, as measured by ELISA (Peprotech). For each assay, a parallel well treated identically was prepared, in which the level of LDH was measured to confirm that drug treatment did not cause cell death.

Mouse CIA Cell Studies.

Mice with CIA were sacrificed, and splenic T cells were isolated with a MACS system and negative selection. Isolated T cells were stimulated with anti-CD3+CD28 Dynabeads in the presence of 0 or 0.1 μM of HCQ and/or 0, 0.1, or 10 μM of atorvastatin for 48 hours, following which culture supernatants were collected and IFN-γ levels measured by ELISA. A combination of HCQ and atorvastatin (Atov.) synergistically reduced anti-CD3+CD28 Dynabead-mediated IFN-γ production (P<0.05 by Tukey test) (FIG. 12).

Mouse EAE Studies.

Mice with PLP-induced EAE were sacrificed, and splenic cells isolated. Isolated splenic cells were stimulated with PLP in the presence of 0 or 0.1 μM of HCQ and/or 0, 0.1, or 10 μM of atorvastatin and/or the presence of 0, 1, 3, or 10 μM of HCQ for 48 hours, following which culture supernatants were collected and levels of IFN-γ and IL-17 measured by ELISA. The Tukey test was used to statistically compare results between groups, and demonstrated that the combination of HCQ 1 μM and atorvastatin 3 μM synergistically inhibited PLP-induced production of the pro-inflammatory cytokines IFN-γ and IL-17 (P<0.05 by the Tukey test).

### Example 12

### The Combinations of Atorvastatin+Hydroxychloroquine, and Atorvastatin+Desethylhydroxychloroquine, Prevented the Development of and Reduced the Severity of Osteoarthritis (OA) in a Mouse Model

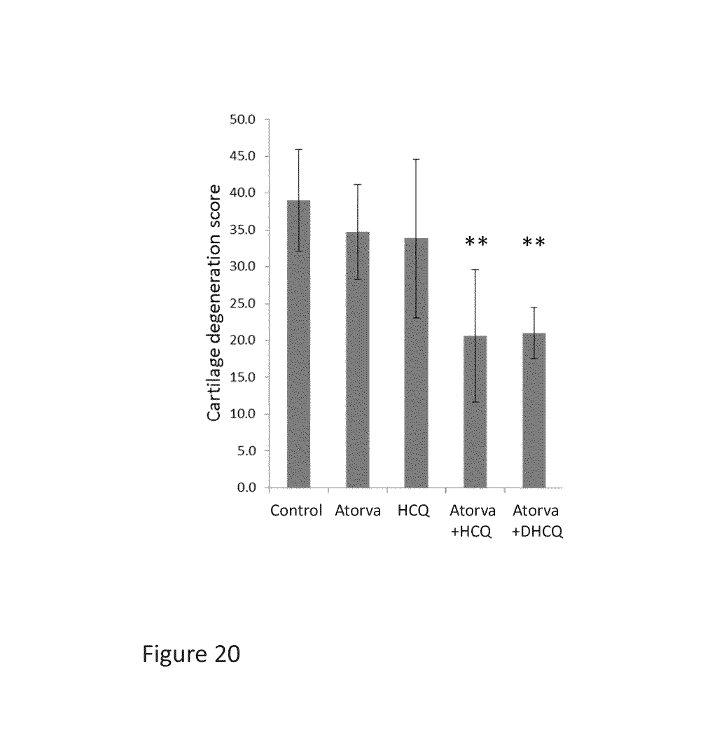

C57BL6 (B6) mice (n=7-10 per group) were surgically induced to develop OA by DMM. One week after surgical induction of DMM, a time at which mice are asymptomatic or have mild joint symptoms and are therefore considered to have pre-clinical OA, treatment was initiated with vehicle (control), atorvastatin calcium 40 mg/kg/day, HCQ sulfate 100 mg/kg/day, the combination of HCQ sulfate 100 mg/kg/day and atorvastatin calcium 40 mg/kg/day, or the combination of DHCQ 100 mg/kg/day and atorvastatin calcium 40 mg/kg/day. All treatments were delivered by oral gavage. After 3 months, mice were sacrificed, their joints harvested, joint sections cut, and tissue sections stained with safranin-O. The mean “Cartilage degeneration scores” in safranin-O stained sections of the medial region of stifle joints are presented in the graph in FIG. 20.

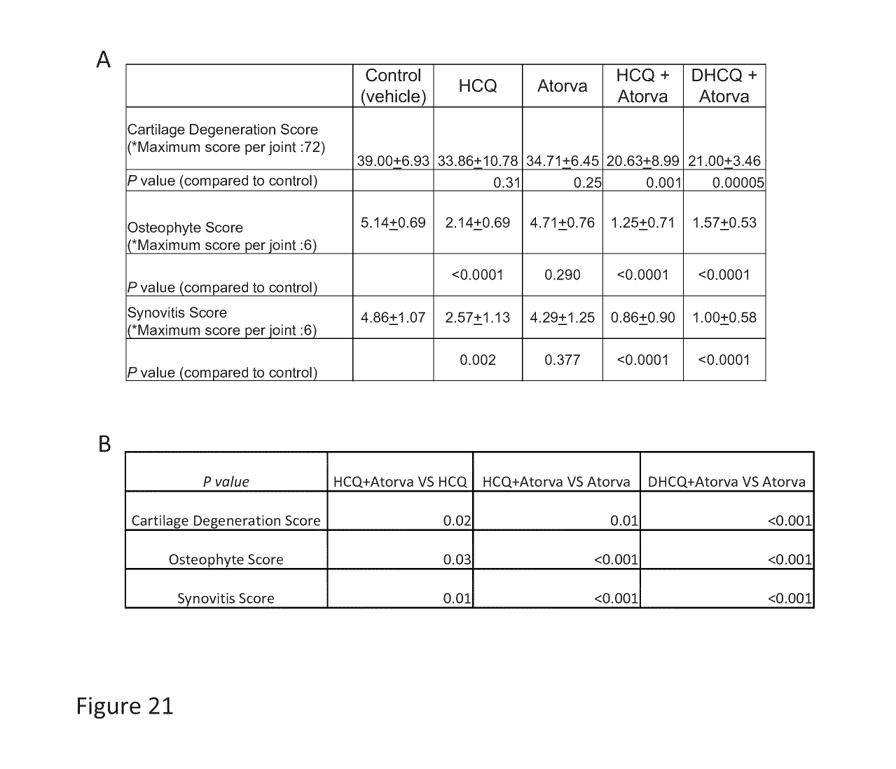

The combination of atorvastatin and HCQ, and the combination of atorvastatin and DHCQ, prevented cartilage degeneration this mouse model of OA. The mean “Cartilage degeneration scores” in safranin-O stained sections of the medial region of stifle joints were compared between the vehicle-treated group and each of the other treatment groups by two-tailed t tests, and it was demonstrated that, compared to treatment with vehicle, the combination of atorvastatin and HCQ, and of atorvastatin and DHCQ, each significantly prevented the development of and reduced the severity of OA (P<0.01) as compared to vehicle-treated mice (FIGS. 20 and 21).

The mean “Cartilage degeneration scores” in safranin-O stained sections of the medial region of stifle joints were also compared between the individual treatment groups (HCQ alone, or atorvastatin alone) and the combination treatment groups (HCQ+atorvastatin, or DHCQ+atorvastatin) by two-tailed t tests (FIG. 21). The combination of atorvastatin and HCQ, as well as the combination of atorvastatin and DHCQ, both significantly prevented the development of, reduced the level of synovitis (inflammation) in, and reduced the severity of OA (P<0.01) as compared to treatment with HCQ alone or with atorvastatin alone.

### Example 13

### Treatment of the Inflammatory Disease Non-Alcoholic Steatohepatitis (NASH) with the Combination of Hydroxychloroquine and Atorvastatin

A 49-year-old man is noted to have elevated liver enzymes with an alanine transaminase (ALT) level of 59 IU/L and an aspartate transaminase (AST) level of 55 IU/L. Results from ultrasound imaging of the liver are consistent with fatty infiltration, serologic tests are negative for HBV or HCV infection, and he denies use of alcohol. He is found to have an IFG level of 120 and elevated levels of triglycerides (323 mg/dL). He undergoes liver biopsy, and analysis of the biopsy demonstrates steatosis ballooning, degeneration of hepatocytes, as well as mixed portal inflammation but no fibrosis. He is diagnosed with NAFLD and early-stage NASH and is prescribed HCQ sulfate 400 mg and atorvastatin calcium 20 mg daily to prevent progression of his disease.

### Example 14

### Treatment of the Inflammatory Disease Type II Diabetes with the Combination of Hydroxychloroquine and Atorvastatin

A 42-year-old man with history of obesity (BMI of 31) is found to have a fasting glucose level of 106 mg/dL, an LDL level of 135, and a triglyceride level of 220. Tests for secondary causes of hyperglycemia are negative, and he is treated with atorvastatin calcium 40 mg and HCQ sulfate 400 mg daily.

A 53-year-old man with a history of hypertension and previous myocardial infarction is noted to have an LDL cholesterol level of 140 mg/dL. He is treated with atorvastatin, which decreases his LDL to 115 mg/d over several months. He is subsequently treated with the combination of HCQ sulfate 400 mg daily and atorvastatin calcium 40 mg daily to more effectively treat and prevent secondary complications from his type II diabetes.

### Example 15

### Treatment of the Chronic Immune Activation in HIV Infection with the Combination of Hydroxychloroquine and Atorvastatin

A 38-year-old man with a 9-year history of HIV infection, treated with a triple-drug regimen of anti-retroviral therapy has an undetectable viral load (<10,000 copies/ml) and CD4+ T-cell count of 490. He feels well and has had no opportunistic infections. He is noted to have an IFG level of 109 mg/dL and elevated levels of triglycerides (299 mg/dL). His hsCRP level is 5.8 mg/L. A coronary CT scan reveals significant calcification of the coronary arteries with an Agatston score of 124, but an exercise stress test reveals no inducible cardiac ischemia. He is prescribed HCQ sulfate 400 mg and atorvastatin calcium 40 mg daily to prevent development of complications from HIV-associated chronic immune activation.

### Example 16

### Treatment of the Inflammatory Disease Atherosclerosis with the Combination of Hydroxychloroquine and Atorvastatin

A 59-year-old man with a history of hypertension is evaluated for levels of cholesterol and inflammatory markers on his annual visit to his primary care physician. The man's cholesterol as 250 mg/dL with an LDL of 165 mg/dL. He has no symptoms. He is treated with HCQ sulfate 350 mg daily and atorvastatin calcium 35 mg daily to prevent development of atherosclerotic coronary artery disease.

### Example 17

### Treatment of the Inflammatory Disease Macular Degeneration with the Combination of Hydroxychloroquine and Atorvastatin

A 74-year-old woman is referred by her primary care doctor to an ophthalmologist for blurry vision. Opthalmological evaluation reveals loss of central vision that is more pronounced in the right than in the left eye, as well as the presence of drusen deposits and degeneration of the retinal pigment epithelium. At a follow-up visit 4 months later it is discovered that the loss of central vision in the right eye has progressed. The patient is prescribed atorvastatin calcium 20 mg daily and HCQ sulfate 200 mg daily to prevent progression of the macular degeneration.

### Example 18

### The Combinations of Atorvastatin and Hydroxychloroquine Inhibited Inflammatory Cytokine Production in OA Synovium in a Mouse Model of OA

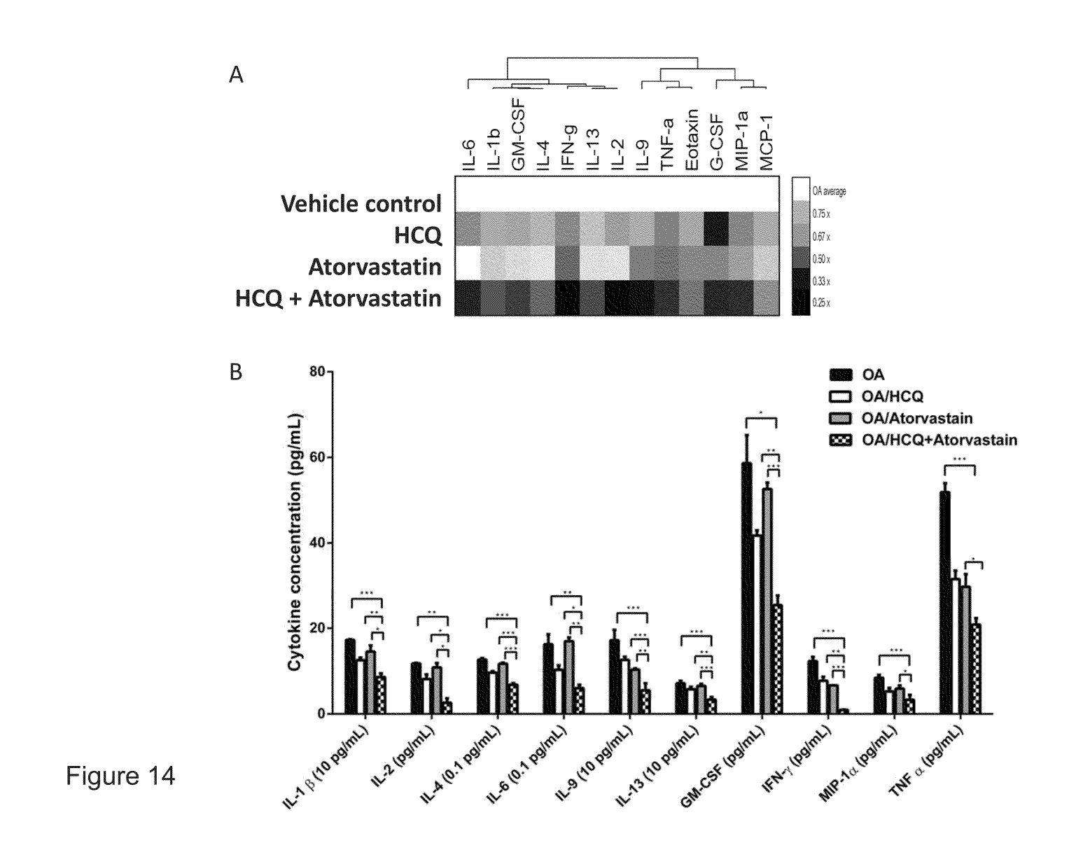

C57BL6 (B6) mice were surgically induced to develop OA by DMM. One week after DMM, a time at which mice are asymptomatic or have mild joint symptoms and are therefore considered to have pre-clinical OA, treatment was initiated with vehicle control, atorvastatin calcium 40 mg/kg/day, HCQ sulfate 100 mg/kg/day, or the combination of atorvastatin calcium 40 mg/kg/day and HCQ sulfate 100 mg/kg/day. After 3 months, mice were sacrificed, their joints harvested, and synovial tissue isolated from the joint by microdissection. The synovial tissues were homogenized and centrifuged, and the resulting supernatants were assayed for levels of inflammatory cytokines by using a multiplex bead-based cytokine assay (BioRad Laboratories, Hercules, Calif.). FIG. 14A shows a heat map representing the relative levels of inflammatory cytokines in mouse OA synovium. Levels of inflammatory cytokines in OA synovium were lower in mice treated with HCQ alone or atorvastatin alone than in mice treated with vehicle. The combination of HCQ+atorvastatin synergistically reduced multiple inflammatory cytokine levels as compared to treatment with HCQ alone or atorvastatin alone (FIG. 14B; *P<0.05, **P<0.01, ***P<0.0.001).

### Example 19

### Analysis of Retinal Pathology Demonstrates that Atorvastatin Reduces the Retinal Toxicity of Hydroxychloroquine

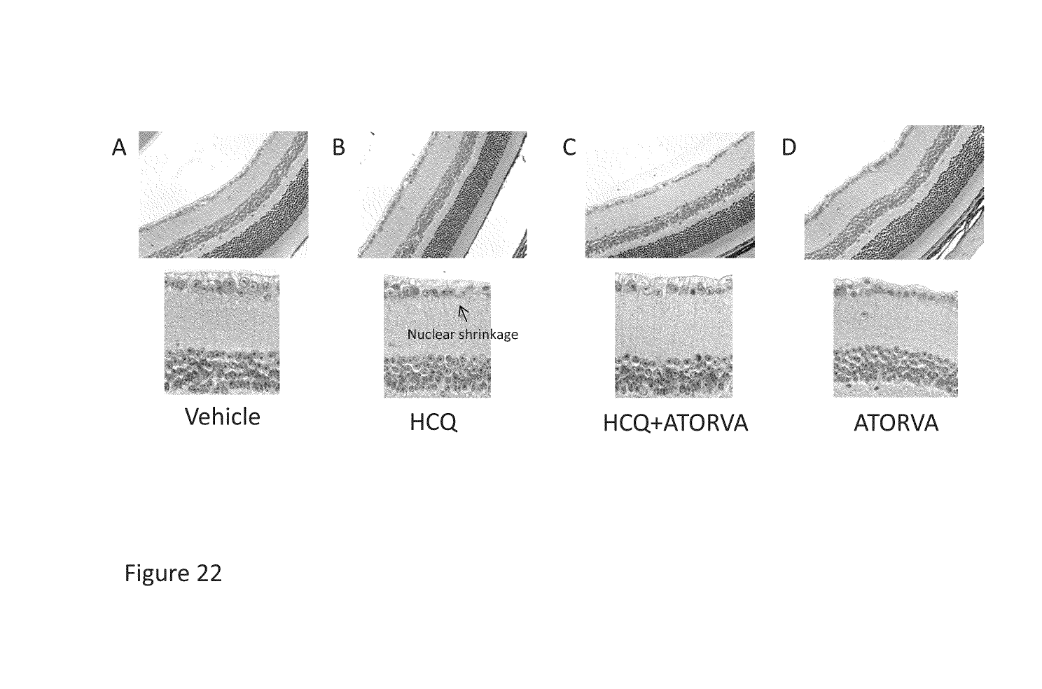

C57BL6 (B6) mice (n=7-10 per treatment group) were treated with vehicle (control), atorvastatin calcium 40 mg/kg/day, HCQ sulfate 100 mg/kg/day, or the combination of atorvastatin calcium 40 mg/kg/day and HCQ sulfate 100 mg/kg/day. After 3 months, mice were sacrificed, and their eyes were carefully isolated by microdissection. The eyes were fixed in formalin and sectioned to allow visualization of the retina. The retinal cell layer was stained with H&E, and the number of nuclei in the ganglion cell layer (GCL) was evaluated, as was nuclear shrinkage in the GCL, a finding suggestive of selective loss and death of retinal ganglion cells. Increased nuclear shrinkage in the GCL was observed in mice treated with HCQ alone, but not in mice treated with the combination of HCQ plus atorvastatin, atorvastatin alone, or with vehicle (FIG. 22). Further, the GCL was disarranged in HCQ-treated mice, while no disarrangement was observed in mice treated with vehicle, the combination of HCQ plus atorvastatin, or atorvastatin alone.

Using a histologic and quantitative pathology methodology adapted from Shichiri, et al (Shichiri, et al, JBC. 2012 287(4):2926-34. PMID 22147702), the H&E-stained retinal sections were evaluated for number of nuclei in the GCL. The number of nuclei in the GCL (which represents the number of ganglion cells in the GCL) was significantly lower in mice treated with HCQ alone as compared to mice treated with vehicle (P≦0.05 by two-tailed t test). Addition of atorvastatin to HCQ resulted in statistical protection against HCQ-mediated GCL cell loss and death (P≦0.01 by two-tailed t test). These results demonstrate that atorvastatin is protective against HCQ-mediated retinal ganglion cell death that results in retinopathy.

Thus, in addition to the synergistic anti-inflammatory activity of the combination of HCQ and atorvastatin, the ability of the component atorvastatin to reduce HCQ-mediated eye toxicity will enable a larger total cumulative dose of HCQ to be delivered over the course of therapy. The higher cumulative dose of HCQ enabled by atorvastatin enables a larger amount of HCQ to be included in each dose of the combination therapy and/or enables the combination therapy to be therapeutically delivered over a longer period of time. Many inflammatory diseases including osteoarthritis, rheumatoid arthritis, type II diabetes, metabolic syndrome, atherosclerosis, osteoarthritis, Alzheimer's disease and others necessitate decades of treatment over an individual's lifetime, and atorvastatin's ability to reduce HCQ-mediated retinal toxicity will enable the HCQ+atorvastatin combination to be therapeutically delivered over years and decades to treat chronic inflammatory disease.

### Example 20

### Demonstration that the Combination of HCQ and Atorvastatin Results in Less HCQ-Mediated Retinal Toxicity in Humans with Inflammatory Disease

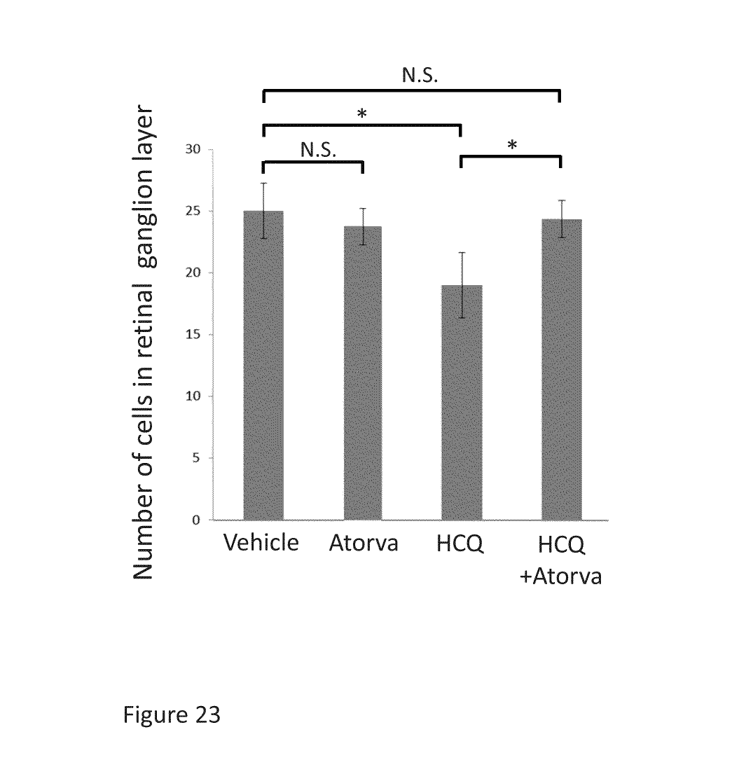

It is demonstrated herein that treatment with the combination of HCQ and atorvastatin reduced HCQ-mediated retinal toxicity in mice (FIGS. 22 and 23). Humans with inflammatory disease are treated with the combination of HCQ and atorvastatin, and starting after 5 years of treatment tested annually for HCQ-mediated retinal toxicity using multifocal electroretinogram (mfERG), spectral domain optical coherence tomography (SD-OCT), fundus autofluorescence (FAF), visual field tests, and/or direct visualization of the macula. HCQ-mediated retinal toxicity is determined based on a worsening of the results (deterioration of performance on the test and/or worsening of retinal or macular findings) on the HCQ-retinal toxicity screening exams. Individuals with inflammatory disease treated with the combination of HCQ and atorvastatin are expected to exhibit a 50% lower rate of retinal toxicity (e.g. worsening of annual screening test results in the exams described in detail below) as compared to that reported for individuals treated with a similar effective total cumulative HCQ dose over a similar period of time. Thus, in a patient population analogous to that described by the American College of Opthamolology and Marmor et al (Ophthalmology. 2011 February; 118(2):415-22), in which the retinal toxicity rate approached 1% after 5 years in individuals treated with HCQ alone, we anticipate that the combination of HCQ+atorvastatin will reduce the rate of HCQ-mediated retinal toxicity to less that 0.5% of treated individuals.

Further, we anticipate that treatment of a group of individuals with the combination of HCQ+atorvastatin will exhibit reduced rates (e.g. incidence) of retinal toxicity as compared to treatment with a similar cumulative dose of HCQ over a 5 year period and over a 10 year period on the following quantitative exams (Marmor, Ophthalmology. 2011 February; 118(2):415-22):

(1) Automated Threshold Visual Fields. Parafoveal loss of visual sensitivity may appear before changes are seen on fundus examination. The finding of any reproducibly depressed central or parafoveal spots is indicative of early toxicity. Advanced toxicity will show a well-developed paracentral scotoma. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of depressed central or parafoveal spots at 5 years, and at 10 years, of treatment as compared to treatment with HCQ alone. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of reproducibly depressed central or parafoveal spots at 5 years, and at 10 years, of treatment as compared to treatment with HCQ alone.

(2) Spectral Domain-Optical Coherence Tomography. High-resolution instruments (SD or Fourier domain OCT) can show localized thinning of the retinal layers in the parafoveal region to demonstrate toxicity. Loss of the inner-/outer-segment line may be an early objective sign of parafoveal damage. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of localized thinning of the retinal layers in the parafoveal region at 5 years, and at 10 years, of treatment as compared to treatment with HCQ alone.

(3) Fundus Autofluorescence. Autofluorescence imaging may reveal subtle RPE defects with reduced autofluorescence or show areas of early photoreceptor damage (which appear as increased autofluorescence from an accumulation of outer segment debris). FAF abnormalities can be detected before visual field loss. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of subtle RPE defects with reduced autofluorescence or areas of early photoreceptor damage at 5 years, and at 10 years, of treatment as compared to treatment with HCQ alone at a similar effective cumulative dose and over a similar time period.

(4) Multifocal Electroretinogram. The mfERG generates local ERG responses topographically across the posterior pole and can objectively document localized paracentral ERG depression in early HCQ retinopathy. The treatment with the HCQ+atorvastatin is anticipated to be associated with at least about a 25%, at least about a 35%, at least about a 45%, at least about a 55%, at least about a 65%, at least about a 75%, and may be around or up to about a 50% lower rate of localized paracentral ERG depression at 5 years, and at 10 years, of treatment as compared to treatment with HCQ alone.

1. A method for treating an individual at increased risk for or in the early stages of an inflammatory disease comprising:
administering to the individual an effective amount of a combination of active agents comprising an aminoquinoline and a statin.

2. The method of claim 1, wherein the individual is asymptomatic and identified as being at increased risk for developing the inflammatory disease.

3. The method of claim 1, wherein the individual is in the early stages of an inflammatory disease.

4. The method of claim 1, wherein the statin comprises atorvastatin, or a salt or ester thereof.

5. The method of claim 1, wherein the statin is atorvastatin, pravastatin, simvastatin, lovastatin, rosuvastatin, fluvastatin, or salts or esters thereof.

6. The method of claim 4, wherein the atorvastatin is administered in an amount of between about 10 and about 40 mg per day (0.1-0.4 mg/kg/day).

7. The method of claim 4, wherein the atorvastatin is administered in an amount of between about 2.5 and about 100 mg per day (0.025-1 mg/kg/day).

8. The method of claim 1, wherein the aminoquinoline comprises hydroxychloroquine, or a salt or ester thereof.

9. The method of claim 8, wherein the hydroxychloroquine is administered in an amount of between about 50 and about 800 mg per day (0.5-6 mg/kg/day).

10. The method of claim 8, wherein the hydroxychloroquine is administered in an amount of between about 25 and about 2000 mg per day (0.25-20 mg/kg/day).

11. The method of claim 8, wherein prior to administering hydroxychloroquine in an amount of between about 25 and about 2000 mg per day (0.25-20 mg/kg/day), a higher loading dose of hydroxychloroquine is first administered for 2 to 16 weeks.

12. (canceled)

13. The method of claim 1, wherein the inflammatory disease is osteoarthritis.

14. The method of claim 1, wherein the inflammatory disease is rheumatoid arthritis or multiple sclerosis or atherosclerosis or non-alcoholic steatohepatitis or type II diabetes or chronic infection or HIV.

15-20. (canceled)

21. The method of claim 1, further comprising identifying the individual as at risk of or in the early stages of an inflammatory disease by a diagnostic test comprising measuring one or more clinical biomarkers, imaging biomarkers, molecular biomarkers, or metabolic biomarkers, and comparing the levels to a control measurement.

22. The method of claim 21, wherein the diagnostic test is measuring one or more clinical markers, and the one or more clinical markers are selected from the group consisting of a family history of the inflammatory disease, swelling on physical examination, tenderness on physical exam, and combinations thereof.

23. The method of claim 21, wherein the diagnostic test is measuring an imaging marker, and the imaging marker is selected from the group consisting of magnetic resonance imaging, ultrasound, computed tomography, angiography, and combinations thereof.

24. The method of claim 21, wherein the diagnostic test is measuring a molecular marker, and the molecular marker is selected from the group consisting of a genetic marker, autoantibody, inflammatory marker, cartilage marker, metabolic marker, bone marker, and combinations thereof.

25. A method of treating an individual at increased risk for developing an inflammatory disease or exhibiting the early stages of an inflammatory disease comprising:
(i) identifying an individual at increased risk for developing or exhibiting early stages of an inflammatory disease,
(ii) measuring a marker of inflammation in the individual,
(iii) comparing the level of the marker of inflammation measured in the individual with the levels of the marker of inflammation measured in healthy individuals to determine if the individual exhibits increased inflammation,
(iv) if the individual exhibits increased inflammation, treating the individual by administering an effective amount of a combination comprising an aminoquinoline and a statin.

26-48. (canceled)

49. A pharmaceutical composition comprising:
an effective dose of an aminoquinoline and a statin for treating inflammation; and a pharmaceutically acceptable excipient.

50-59. (canceled)
